SEQUENCE LISTING (37 sequences)

SEQ ID NO: 1 - DNA, 13242 bp, Human foamy virus
tgtggtggaatgccactagaaactagggaaaactaggaggagagtattacagggaaggaagtgaagaacctcgtgacccaaatactcctgctcctcatagacgtacctgggatgagagacacaaggttcttaaattgtcctcattcgctactccctctgacatccaacgctgggctactaaagcattgccttatggctggaaagtggtcaccgaaagcggaaatgattatactagccgcagaaagatcagaacattgacagagatgactcaggatgaaattagaaaaaggtgggaaagtggatattgtgaccccttcattgactcaggaagtgactcagatggacccttctaaaagccacagacagtaaaaatgtgttagcactttatacaatattatatctgcttaagctatagaagctttcacatactcagtagctgtttcacaatcaacaaaacaatgatgatgtaatcataaggaagtagtttaaaataggttaagtaagtttactgcagtagataatccctggggaggatctggctctgtaagctggaacagcaatgttttcagttccaatcctctcaaaggagaaccagagggatgatgtgttagttcaaatcccattatcctcatggttcccttttccatagtttactatattaatttaaggataaggttatatatgctttgctatccctgaaggaagcatagctcttcaggtaaagaggccagtattaaaagagctacccttccttttcatataaggatcgaaacctgcttacgttttactttaataaagttaagttttaagttttaacaggaaatgctcttaggcagaaggtagctccctcgactgggtgacaaggaagaaaccattcaggaagtgcttccttctgctcggggagatacatgagtatacagtagttcgaatcctgtgtgctgatgatgtcttctcggctttttgtgacatcacaaatgttacttagaggtagctttctgagaggtttatacctcagaaggagtgtggctaatgctggtaaggcacctttctatcagtacaatcaagcccattcctccccatgttctcagggtgtgtgcggcgtaagatcgaatccccacacacctgggaacttgcttattgcataataattttattagtcatatagaaaataatataggataaggtataaggattaaggtatgaggtgtgtggctcaacacgtagggtgacaagaaaatctactgtaataggacacaacacctctaaagttgcccgtgggaaggtgaagtgagatcgaatctttccttaacgcagacagctttttatccactagggataatgttttaaggaatactataggaatagattgatagttttaacaatgatagaaatagtatatagggatagtttctagattgtacgggagctcttcactactcgctgcgtcgagagtgtacgagactctccaggtttggtaagaaatattttatattgttataatgttactatgatccattaacactctgcttatagattgtaagggtgattgcaatgctttctgcataaaactttggttttcttgttaatcaataaaccgacttgattcgagaacctactcatatattattgtctcttttatactttattaagtaaaaggatttgtatattagccttgctaagggagacatctagtgatataagtgtgaactacacttatcttaaatgatgtaactccttaggataatcaatatacaaaattccatgacaattggcgcccaacgtggggctcgaatataagtcgggtttatttgtaaattatccctagggacctccgagcatagcgggaggcatataaaagccaatagacaatggcttcaggaagtaatgttgaagaatatgaacttgatgttgaagctctggttgtaattttaagagatagaaatataccaagaaatcctttacatggagaagttataggtcttcgccttactgaaggatggtggggacaaattgagagatttcagatggtacgtctaatattacaagatgatgataatgaacctttacagagacctagatatgaggtaatacaacgagctgtaaaccctcatacaatgtttatgatatcaggaccattagctgaacttcaattagcctttcaggatttagatttacctgaaggtccattgaggtttggtccattggcaaatggacattatgttcaaggagatccttatagtagttcttacagaccagtaacaatggccgaaacagcccaaatgactagagatgaactggaagatgttcttaatactcaaagtgaaatagaaattcaaatgataaatttattggagttgtatgaagttgaaactagagctcttagaagacaattagctgagagatctagtacagggcaaggaggaatatccccaggagctcctcgttctcgaccaccagtaagcagcttctcagggttaccaagtttgccctctatacctgggattcatcccagggcaccttcacctccaagggcaacttctactcccggaaatattccttggagtttaggagatgatagcccaccttcatctagttttcctggaccctctcaacctcgtgtttctttccatccgggaaatccttttgttgaagaagaaggtcatagacctagatcccagtctagagaaaggagaagagaaattcttcctgctcctgtaccgtcagcacctcctatgattcagtatataccagtaccacctccaccaccgattggcacggttatacctattcagcatatcagatctgtaactggagagcctcctagaaacccaagagaaataccaatttggctaggacgaaatgctcctgctatagatggagtgttccctgttacaacaccggatctaagatgcagaataattaatgctatactaggaggaaatattgggctatcattaacccctggagactgtttaacatgggactcagcagtagccaccttatttattagaacccatggaacttttccaatgcatcagcttggaaatgtaataaaaggcatagttgatcaagaaggagtggcaacagcatatactttgggaatgatgctttctggacaaaattatcaattagtttctggaataattagaggatatttgcctggacaagctgtagtaactgcattacaacagcgtttagaccaagaaatagatgatcaaacaagagcagagacttttattcaacatctaaatgctgtatatgaaattttaggccttaatgccagaggacaaagtatacgtgcttcagtgactcctcaaccccgaccatccagaggtagaggtcgaggtcaaaatacttctagaccctctcaaggaccagctaatagcgggcggggacgacagcgccctgcttctggtcaaagcaacagaggatctagtactcagaatcaaaatcaagataatttaaatcaaggaggatataatcttcgaccccgtacttaccaacctcaaaggtacggaggaggacgtggacgaagatggaacgataatactaacaatcaagagtccagaccatcagatcaaggttctcaaactcctaggccaaatcaagcaggctctggggtgcgtggcaatcagtcacaaactcccagaccagctgctggtcgcggaggaagaggtaaccacaaccgaaaccaacgatcatccggtgctggtgactcacgcgctgtcaataccgtgacacagagtgccacgtcctccacagatgaatcctcttcagctgttacagccgcttccggcggagatcaaagggactaaattgttagcccactgggattcaggggcaacaataacttgtattcctgaaagttttttagaagatgaacaacctattaaaaagactttaataaaaacaattcatggagaaaaacaacaaaatgtttattatgtaacctttaaagttaaaggaagaaaagtggaagcagaagtgatagcttctccttatgagtatattttgctgtcgccaacagatgttccttggttaacacagcaaccacttcagttaacaattttagttcctcttcaagaatatcaagagaaaatcttaagtaagactgctcttccagaagatcaaaaacaacaattaaaaaccttgtttgtcaagtatgacaatctatggcaacattgggaaaatcaagtcgggcatagaaaaattaggccacataatatagcaactggtgattatcctcctcgccctcaaaaacaatatcctattaatcctaaggcaaagcctagtatacaaattgtaatagatgacttattgaaacaaggggtgttaacgcctcaaaatagtacaatgaatacaccagtgtatcctgttcctaaaccagatggaaggtggagaatggtattagattatagagaagtaaataaaactattccattaacagctgcccaaaaccaacactctgctggtattttagctactattgttagacaaaaatataaaactaccttagatttagctaatggattttgggctcatcctattacaccagaatcttattggttaacagcatttacctggcaaggtaaacagtattgttggacacgtcttcctcaaggatttttaaatagtccagcattgtttacagctgatgtagtagatttactaaaagaaatccctaatgtacaagtgtatgttgatgatatatatttaagccatgatgatcctaaagagcatgttcaacaattagaaaaagtgtttcaaattttactacaggcaggatatgtagtatctttgaaaaaatcagaaattggtcaaaaaactgtagaatttttaggatttaatattactaaagaaggtcgtggcctaacagacacttttaaaacaaaactgttaaatattactcctccaaaagacttaaagcaattacaaagcatattaggattgttaaattttgctagaaattttatacctaattttgctgaactggtacaaccattatacaatttaatagcctcagcaaaaggcaaatatattgagtggtctgaagaaaatactaaacaattaaatatggtaatagaagcattaaacactgcctctaatttagaagaaaggttaccagaacagagactggtaattaaagtcaatacttctccatcagcaggatatgtaagatattataatgagactggtaaaaagcctattatgtacctaaattatgtgttttccaaagcagaattaaaattttctatgttagaaaaactattaactacaatgcacaaagccttaattaaggctatggatttggccatgggacaagaaatattagtttatagtcccattgtatctatgactaaaatacaaaaaactccactaccagaaagaaaagctttacccattagatggataacatggatgacttatttagaagatccaagaatccaatttcattatgataaaaccttaccagaacttaagcatattccagatgtatatacatctagtcagtctcctgttaaacatccttctcaatatgaaggagtgttttatactgatggctcggccatcaaaagtcctgatcctacaaaaagcaataatgctggcatgggaatagtacatgccacatacaaacctgaatatcaagttttgaatcaatggtcaataccactaggtaatcatactgctcagatggctgaaatagctgcagttgaatttgcctgtaaaaaagctttaaaaatacctggtcctgtattagttataactgatagtttctatgtagcagaaagtgctaataaagaattaccatactggaaatctaatgggtttgttaataataagaaaaagcctcttaaacatatctccaaatggaaatctattgctgagtgtttatctatgaaaccagacattactattcaacatgaaaaaggcatcagcctacaaataccagtattcatactgaaaggcaatgccctagcagataagcttgccacccaaggaagttatgtggttaattgtaataccaaaaaaccaaacctggatgcagagttggatcaattattacagggtcattatataaaaggatatcccaaacaatatacatattttttagaagatggcaaagtaaaagtttccagacctgaaggggttaaaattattccccctcagtcagacagacaaaaaattgtgcttcaagcccacaatttggctcacaccggacgtgaagccactcttttaaaaattgccaacctttattggtggccaaatatgagaaaggatgtggttaaacaactaggacgctgtcaacagtgtttaatcacaaatgcttccaacaaagcctctggtcctattctaagaccagataggcctcaaaaaccttttgataaattctttattgactatattggacctttgccaccttcacagggatacctatatgtattagtagttgttgatggaatgacaggattcacttggttataccccactaaggctccttctactagcgcaactgttaaatctctcaatgtactcactagtattgcaattccaaaggtgattcactctgatcaaggtgcagcattcacttcttcaacctttgctgaatgggcaaaggaaagaggtatacatttggaattcagtactccttatcacccccaaagtggtagtaaggtggaaaggaaaaatagtgatataaaacgacttttaactaaactgctagtaggaagacccacaaagtggtatgacctattgcctgttgtacaacttgctttaaacaacacctatagccctgtattaaaatatactccacatcaactcttatttggtatagattcaaatactccatttgcaaatcaagatacacttgacttgaccagagaagaagaactttctcttttacaggaaattcgtacttctttataccatccatccacccctccagcctcctctcgttcctggtctcctgttgttggccaattggtccaggagagggtggctaggcctgcttctttgagacctcgttggcataaaccgtctactgtacttaaggtgttgaatccaaggactgttgttattttggaccatcttggcaacaacagaactgtaagtatagataatttaaaacctacttctcatcagaatggcaccaccaatgacactgcaacaatggatcatttggaaaaaaatgaataaagcgcatgaggcacttcaaaatacaacaactgtgactgaacagcagaaggaacaaattatactggacattcaaaatgaagaagtacaaccaactaggagagataaatttagatatctgctttatacttgttgtgctactagctcaagagtattggcctggatgtttttagtttgtatattgttaatcattgttttggtttcatgctttgtgactatatccagaatacaatggaataaggatattcaggtattaggacctgtaatagactggaatgttactcaaagagctgtttatcaacccttacagactagaaggattgcacgttcccttagaatgcagcatcctgttccaaaatatgtggaggtaaatatgactagtattccacaaggtgtatactatgaaccccatccggaacccatagtggtgaaggagagggtcctaggtctttctcaaattctgatgattaattcagaaaacattgctaataatgctaatttgacacaagaagtaaagaagttgttaactgaaatggttaatgaagaaatgcaaagtttgtcagatgtaatgattgactttgaaattcctttaggagaccctcgtgatcaagaacaatatatacatagaaaatgctatcaagaatttgcaaattgttatttagtaaaatataaagaacccaaaccgtggcctaaggagggccttatagctgatcaatgcccattaccaggttaccatgctggattaacctataatagacagtctatttgggattactatattaaagtggagagtattagacctgcaaattggacaacaaagagtaaatatggacaagctagactaggaagtttttatattcctagcagcctgagacaaatcaatgttagtcatgtactattctgtagtgatcaattatattctaaatggtataatatagaaaataccatagaacaaaacgagcggtttctgcttaataaactaaataaccttacatctggaacctcagtattgaagaaaagagctcttccgaaggattggagttctcaaggtaaaaatgctctgtttagagaaatcaatgtgttagatatctgcagtaaacctgaatctgtaatactattgaatacttcatactattccttctctttatgggaaggagattgtaattttactaaagatatgatttctcagttggttccagaatgtgatggattttataacaattctaagtggatgcatatgcatccatatgcttgtagattctggagaagtaagaatgaaaaagaagaaactaaatgtagagatggggaaactaagagatgtctgtattatcctttatgggacagtcccgaatctacatatgattttggttatttagcataccaaaagaattttccttcccctatctgtatagaacaacagaaaattagagatcaagattatgaagtctattctttgtatcaagaacgcaaaatagcttctaaagcatatggaattgatacagttttattctctctaaagaattttcttaattatacaggaactcctgtaaatgaaatgcctaatgcaagagcttttgtaggcctaatagatcccaagtttcctccttcctatcccaatgttactagggaacattatacttcctgtaataataggaaaagaagaagtgttgataataactatgctaagttaaggtctatggggtatgcacttacaggagcagtgcaaaccttatctcaaatatcagatattaatgatgaaaacttacagcaaggaatatatttattaagggatcatgtaataaccttaatggaagctacattgcatgatatatctgttatggaaggaatgtttgctgtacaacatttgcatacacatttgaatcatttgaagacaatgcttctagaaagaagaatagactggacctatatgtctagtacttggctacaacaacaattacagaaatctgatgatgagatgaaagtaataaagagaattgctagaagtttggtatattatgttaaacaaacccatagttctcccacagctacagcctgggagattggattatattatgaattggttatacctaaacatatttacttgaataattggaatgttgtcaatataggtcacttagttaaatcagctggacaattgactcatgtaactatagctcatccttatgaaataatcaataaggaatgtgtagagactatatatctgcatcttgaggactgcacaagacaagattatgtcatatgtgatgtggtaaagatagtgcagccttgtggcaatagctcagacacgagtgattgtcctgtctgggctgaagctgtaaaagaaccatttgtgcaagtcaatcctctgaaaaacggaagttatctggttttggcaagttccacagactgtcagatcccaccatatgttcctagcatcgtgactgttaatgaaacaacgtcatgctttggactggactttaaaaggccactggttgcggaagaaagattgagctttgagccacgactgccaaatctacaactaagattaccacatttggttggaattattgcaaaaatcaaagggataaaaatagaagtcacatcctctggagaaagtataaaagagcagattgaaagagcaaaagctgagctccttcgactggacattcacgagggagatactcctgcctggatacaacagctagctgcagcaacaaaggacgtctggccagcagcagcttctgctctacaaggaattggtaactttttatctgggactgcccaaggaatatttggaactgcctttagtctcttgggatacttaaagcctatcctaataggagtaggggtcattctcttggttattcttatatttaagattgtatcatggattcctacgaaaaagaagaatcagtagcctccacctctggaattcaggacctgcagactctgagtgagcttgttggccctgaaaatgccggagagggagagctgactattgctgaggaacctgaagaaaatcctcgacgccccagacgatatactaaaagagaagtcaaatgtgtgtcttatcatgcatataaagaaattgaggacaaacatcctcaacatattaaactgcaggattggatccccacaccagaggaaatgagtaagtcactctgtaaaagacttattttatgtggattgtatagtgcagaaaaggcctcagagattttaaggatgccttttacagtttcttgggaacaatcagatactgaccctgactgttttattgtaagctatacatgtatattttgtgatgctgtaatacatgatcccatgcccataagatgggatcctgaagttggaatttgggtaaaatataaacccctcagaggaattgttggatctgctgtgtttattatgcataaacatcaaagaaactgttctcttgttaaaccttctaccagtcgctcagaaggtccaaaaccaagacctaggcacgatcctgtccttcgatgtgacatgtttgaaaagcatcacaagcctcggcagaaacgacccaggagacgatccatcgataatgagtcatgtgcttccagtagtgacaccatggccaatgagccaggatcactatgcaccaaccctctttggaatcctggaccgctactatcagggctacttgaagagtccagcaacctaccaaacttggaagttcacatgtcaggtggacccttctgggaagaggtttatggggactcaattttgggtcccccctctgggtcaggtgaacattcagttttataagaattatcagattctaacttgctgtcaggctgtagacccatttgctaatatttttcatggtactgatgaagaaatgtttgacattgattcaggtcctgatgtttggtgttctccctctttgtgtttcaaggtaatttatgaaggggcaatgggccaaaagcaagaacaaaaaacatggctgtgcagactaggacatggtcatcgtatgggagcatgcgattaccgtaaagtagatctgtatgcaatgagacaaggaaaagaaaacccttatggagataggggtgatgcagctttgcaatatgcttatcaggttaaaaggggctgtaaagcagggtgcttggcatcacctgtacttaactacaaagctttgcagtttcatagaaccattatggcagacttcaccaatcctaggattggagaaggacatcttgctcatggttaccaagcagctatggaagcttatggacctcagagaggaagtaacgaggagagggtgtggtggaatgtcactagaaaccagggaaaacaaggaggagagtattacagggaaggaggtgaagaacctcattacccaaatactcctgctcctcatagacgtacctgggatgagagacacaaggttcttaaattgtcctcattcgctactccctctgacatccaacgctgggctactaaagcattgccttatggctggaaagtggtcaccgaaagcggaaatgattatactagccgcagaaagatcagaacattgacagagatgactcaggatgaaattagaaaaaggtgggaaagtggatattgtgaccccttcattgactcaggaagtgactcagatggacccttctaaaagccacagacagtaaaaatgtgttagcactttatacaatattatatctgcttaagctatagaagctttcacatactcagtagctgtttcacaatcaacaaaacaatgatgatgtaatcataaggaagtagtttaaaataggttaagtaagtttactgcagtagataatccctggggaggatctggctctgtaagctggaacagcaatgttttcagttccaatcctctcaaaggagaaccagagggatgatgtgttagttcaaatcccattatcctcatggttcccttttccatagtttactatattaatttaaggataaggttatatatgctttgctatccctgaaggaagcatagctcttcaggtaaagaggccagtattaaaagagctacccttccttttcatataaggatcgaaacctgcttacgttttactttaataaagttaagttttaagttttaacaggaaatgctcttaggcagaaggtagctccctcgactgggtgacaaggaagaaaccattcaggaagtgcttccttctgctcggggagatacatgagtatacagtagttcgaatcctgtgtgctgatgatgtcttctcggctttttgtgacatcacaaatgttacttagaggtagctttctgagaggtttatacctcagaaggagtgtggctaatgctggtaaggcacctttctatcagtacaatcaagcccattcctccccatgttctcagggtgtgtgcggcgtaagatcgaatccccacacacctgggaacttgcttattgcataataattttattagtcatatagaaaataatataggataaggtataaggattaaggtatgaggtgtgtggctcaacacgtagggtgacaagaaaatctactgtaataggacacaacacctctaaagttgcccgtgggaaggtgaagtgagatcgaatctttccttaacgcagacagctttttatccactagggataatgttttaaggaatactataggaatagattgatagttttaacaatgatagaaatagtatatagggatagtttctagattgtacgggagctcttcactactcgctgcgtcgagagtgtacgagactctccaggtttggtaagaaatattttatattgttataatgttactatgatccattaacactctgcttatagattgtaagggtgattgcaatgctttctgcataaaactttggttttcttgttaatcaataaaccgacttgattcgagaaccaactcctatattattgtctcttttatactttattaagtaaaaggatttgtatattagccttgctaagggagacatctagtgatataagtgtgaactacacttatcttaaatgatgtaactccttaggataatcaatatacaaaattccatgacaa

SEQ ID NO: 2 - DNA, 1266 bp, Human foamy virus
aggaaaagaagaagtgttgataataactatgctaagttaaggtctatggggtatgcacttacaggagcagtgcaaaccttatctcaaatatcagatattaatgatgaaaacttacagcaaggaatatatttattaagggatcatgtaataaccttaatggaagctacattgcatgatatatctgttatggaaggaatgtttgctgtacaacatttgcatacacatttgaatcatttgaagacaatgcttctagaaagaagaatagactggacctatatgtctagtacttggctacaacaacaattacagaaatctgatgatgagatgaaagtaataaagagaattgctagaagtttggtatattatgttaaacaaacccatagttctcccacagctacagcctgggagattggattatattatgaattggttatacctaaacatatttacttgaataattggaatgttgtcaatataggtcacttagttaaatcagctggacaattgactcatgtaactatagctcatccttatgaaataatcaataaggaatgtgtagagactatatatctgcatcttgaggactgcacaagacaagattatgtcatatgtgatgtggtaaagatagtgcagccttgtggcaatagctcagacacgagtgattgtcctgtctgggctgaagctgtaaaagaaccatttgtgcaagtcaatcctctgaaaaacggaagttatctggttttggcaagttccacagactgtcagatcccaccatatgttcctagcatcgtgactgttaatgaaacaacgtcatgctttggactggactttaaaaggccactggttgcggaagaaagattgagctttgagccacgactgccaaatctacaactaagattaccacatttggttggaattattgcaaaaatcaaagggataaaaatagaagtcacatcctctggagaaagtataaaagagcagattgaaagagcaaaagctgagctccttcgactggacattcacgagggagatactcctgcctggatacaacagctagctgcagcaacaaaggacgtctggccagcagcagcttctgctctacaaggaattggtaactttttatctgggactgcccaaggaatatttggaactgcctttagtctcttgggatacttaaagcctatcctaataggagtaggggtcattctcttggttattcttatatttaagattgtatcatggattcctacgaaaaagaagaatcagtag

SEQ ID NO: 3 - protein, 421 aa, Human foamy virus
RKRRSVDNNYAKLRSMGYALTGAVQTLSQISDINDENLQQGIYLLRDHVITLMEATLHDISVMEGMFAVQHLHTHLNHLKTMLLERRIDWTYMSSTWLQQQLQKSDDEMKVIKRIARSLVYYVKQTHSSPTATAWEIGLYYELVIPKHIYLNNWNVVNIGHLVKSAGQLTHVTIAHPYEIINKECVETIYLHLEDCTRQDYVICDVVKIVQPCGNSSDTSDCPVWAEAVKEPFVQVNPLKNGSYLVLASSTDCQIPPYVPSIVTVNETTSCFGLDFKRPLVAEERLSFEPRLPNLQLRLPHLVGIIAKIKGIKIEVTSSGESIKEQIERAKAELLRLDIHEGDTPAWIQQLAAATKDVWPAAASALQGIGNFLSGTAQGIFGTAFSLLGYLKPILIGVGVILLVILIFKIVSWIPTKKKNQ

SEQ ID NO: 4 - DNA, 378 bp, Human foamy virus
atggcaccaccaatgacactgcaacaatggatcatttggaaaaaaatgaataaagcgcatgaggcacttcaaaatacaacaactgtgactgaacagcagaaggaacaaattatactggacattcaaaatgaagaagtacaaccaactaggagagataaatttagatatctgctttatacttgttgtgctactagctcaagagtattggcctggatgtttttagtttgtatattgttaatcattgttttggtttcatgctttgtgactatatccagaatacaatggaataaggatattcaggtattaggacctgtaatagactggaatgttactcaaagagctgtttatcaacccttacagactagaaggattgcacgt

SEQ ID NO: 5 - protein, 126 aa, Human foamy virus
MAPPMTLQQWIIWKKMNKAHEALQNTTTVTEQQKEQIILDIQNEEVQPTRRDKFRYLLYTCCATSSRVLAWMFLVCILLIIVLVSCFVTISRIQWNKDIQVLGPVIDWNVTQRAVYQPLQTRRIAR

SEQ ID NO: 6 - protein, 408 aa, Human foamy virus
RKRRSVDNNYAKLRSMGYALTGAVQTLSQISDINDENLQQGIYLLRDHVITLMEATLHDISVMEGMFAVQHLHTHLNHLKTMLLERRIDWTYMSSTWLQQQLQKSDDEMKVIKRIARSLVYYVKQTHSSPTATAWEIGLYYELVIPKHIYLNNWNVVNIGHLVKSAGQLTHVTIAHPYEIINKECVETIYLHLEDCTRQDYVICDVVKIVQPCGNSSDTSDCPVWAEAVKEPFVQVNPLKNGSYLVLASSTDCQIPPYVPSIVTVNETTSCFGLDFKRPLVAEERLSFEPRLPNLQLRLPHLVGIIAKIKGIKIEVTSSGESIKEQIERAKAELLRLDIHEGDTPAWIQQLAAATKDVWPAAASALQGIGNFLSGTAQGIFGTAFSLLGYLKPILIGVGVILLVILIF

SEQ ID NO: 7 - DNA, 1721 bp, Influenza virus
atggagaaaatagtgcttctttttgcaatagtcagtcttgttaaaagtgatcagatttgcattggttaccatgcaaacaactcgacagagcaggttgacacaataatggaaaagaacgttactgttacacatgcccaagacatactggaaaagacacacaatgggaagctctgcgatctagatggagtgaagcctctaattttgagagattgtagtgtagctggatggctcctcggaaacccaatgtgtgacgaattcatcaatgtgccggaatggtcttacatagtggagaaggccaatccagtcaatgacctctgttacccaggggatttcaatgactatgaagaattgaaacacctattgagcagaataaaccattttgagaaaattcagatcatccccaaaagttcttggtccagtcatgaagcctcattgggggtgagctcagcatgtccataccagggaaagtcctcctttttcagaaatgtggtatggcttatcaaaaagaacagtacatacccaacaataaagaggagctacaataataccaaccaagaagatcttttggtactgtgggggattcaccatcctaatgatgcggcagagcagacaaagctctatcaaaacccaaccacctatatttccgttgggacatcaacactaaaccagagattggtaccaagaatagctactagatccaaagtaaacgggcaaagtggaaggatggagttcttctggacaattttaaaaccgaatgatgcaatcaacttcgagagtaatggaaatttcattgctccagaatatgcatacaaaattgtcaagaaaggggactcaacaattatgaaaagtgaattggaatatggtaactgcaacaccaagtgtcaaactccaatgggggcgataaactctagcatgccattccacaatatacaccctctcaccatcggggaatgccccaaatatgtgaaatcaaacagattagtccttgcgactgggctcagaaatacccctcaaagagagagaagaggattatttggagctatagcaggttttatagagggaggatggcagggaatggtagatggttggtatgggtaccaccatagcaacgagcaggggagtgggtacgctgcagacaaagaatccactcaaaaggcaatagatggagtcaccaataaggtcaactcgattattgacaaaatgaacactcagtttgaggccgttggaagggaatttaacaacttagaaaggagaatagagaatttaaacaagaagatggaagacgggttcctagatgtctggacttataatgctgaacttctagttctcatggaaaatgagagaactctagactttcatgactcaaatgtcaagaacctttacgacaaggtccgactacagcttagggataatgcaaaggagctgggtaacggttgtttcgagttctatcataaatgtgataatgaatgtatggaaagtgtaagaaacggaacgtatgactacccgcagtattcagaagaagcaagactaaaaagagaggaaataagtggagtaaaattggaatcaataggaatttaccaaatattgtcaatttattctacagtggcgagctccctagcactggcaatcatggtagctggtctatccttatggatgtgctccaatgggtcgttacaatgcagaatttgcatttaaatttgtgagttcagattgtagttaaa

SEQ ID NO: 8 - protein, 564 aa, Influenza virus
MEKIVLLFAIVSLVKSDQICIGYHANNSTEQVDTIMEKNVTVTHAQDILEKTHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQGKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNTPQRERRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGIYQILSIYSTVASSLALAIMVAGLSLWMCSNGSLQCRICI

SEQ ID NO: 9 - DNA, 2277 bp, Artificial Sequence
  HA1-gp47 fusion
atggagaaaatagtgcttctttttgcaatagtcagtcttgttaaaagtgatcagatttgcattggttaccatgcaaacaactcgacagagcaggttgacacaataatggaaaagaacgttactgttacacatgcccaagacatactggaaaagacacacaatgggaagctctgcgatctagatggagtgaagcctctaattttgagagattgtagtgtagctggatggctcctcggaaacccaatgtgtgacgaattcatcaatgtgccggaatggtcttacatagtggagaaggccaatccagtcaatgacctctgttacccaggggatttcaatgactatgaagaattgaaacacctattgagcagaataaaccattttgagaaaattcagatcatccccaaaagttcttggtccagtcatgaagcctcattgggggtgagctcagcatgtccataccagggaaagtcctcctttttcagaaatgtggtatggcttatcaaaaagaacagtacatacccaacaataaagaggagctacaataataccaaccaagaagatcttttggtactgtgggggattcaccatcctaatgatgcggcagagcagacaaagctctatcaaaacccaaccacctatatttccgttgggacatcaacactaaaccagagattggtaccaagaatagctactagatccaaagtaaacgggcaaagtggaaggatggagttcttctggacaattttaaaaccgaatgatgcaatcaacttcgagagtaatggaaatttcattgctccagaatatgcatacaaaattgtcaagaaaggggactcaacaattatgaaaagtgaattggaatatggtaactgcaacaccaagtgtcaaactccaatgggggcgataaactctagcatgccattccacaatatacaccctctcaccatcggggaatgccccaaatatgtgaaatcaaacagattagtccttgcgactgggctcagaaatacccctcaaaggaaaagaacaagtgttgataataactatgctaagttaaggtctatggggtatgcacttacaggagcagtgcaaaccttatctcaaatatcagatattaatgatgaaaacttacagcaaggaatatatttattaagggatcatgtaataaccttaatggaagctacattgcatgatatatctgttatggaaggaatgtttgctgtacaacatttgcatacacatttgaatcatttgaagacaatgcttctagaaagaagaatagactggacctatatgtctagtacttggctacaacaacaattacagaaatctgatgatgagatgaaagtaataaagagaattgctagaagtttggtatattatgttaaacaaacccatagttctcccacagctacagcctgggagattggattatattatgaattggttatacctaaacatatttacttgaataattggaatgttgtcaatataggtcacttagttaaatcagctggacaattgactcatgtaactatagctcatccttatgaaataatcaataaggaatgtgtagagactatatatctgcatcttgaggactgcacaagacaagattatgtcatatgtgatgtggtaaagatagtgcagccttgtggcaatagctcagacacgagtgattgtcctgtctgggctgaagctgtaaaagaaccatttgtgcaagtcaatcctctgaaaaacggaagttatctggttttggcaagttccacagactgtcagatcccaccatatgttcctagcatcgtgactgttaatgaaacaacgtcatgctttggactggactttaaaaggccactggttgcggaagaaagattgagctttgagccacgactgccaaatctacaactaagattaccacatttggttggaattattgcaaaaatcaaagggataaaaatagaagtcacatcctctggagaaagtataaaagagcagattgaaagagcaaaagctgagctccttcgactggacattcacgagggagatactcctgcctggatacaacagctagctgcagcaacaaaggacgtctggccagcagcagcttctgctctacaaggaattggtaactttttatctgggactgcccaaggaatatttggaactgcctttagtctcttgggatacttaaagcctatcctaataggagtaggggtcattctcttggttattcttatatttaagattgtatcatggattcctacgagcagcagcaatcag

SEQ ID NO: 10 - protein, 759 aa, Artificial Sequence
  HA1-gp47 fusion
MEKIVLLFAIVSLVKSDQICIGYHANNSTEQVDTIMEKNVTVTHAQDILEKTHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQGKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNTPQRKRTSVDNNYAKLRSMGYALTGAVQTLSQISDINDENLQQGIYLLRDHVITLMEATLHDISVMEGMFAVQHLHTHLNHLKTMLLERRIDWTYMSSTWLQQQLQKSDDEMKVIKRIARSLVYYVKQTHSSPTATAWEIGLYYELVIPKHIYLNNWNVVNIGHLVKSAGQLTHVTIAHPYEIINKECVETIYLHLEDCTRQDYVICDVVKIVQPCGNSSDTSDCPVWAEAVKEPFVQVNPLKNGSYLVLASSTDCQIPPYVPSIVTVNETTSCFGLDFKRPLVAEERLSFEPRLPNLQLRLPHLVGIIAKIKGIKIEVTSSGESIKEQIERAKAELLRLDIHEGDTPAWIQQLAAATKDVWPAAASALQGIGNFLSGTAQGIFGTAFSLLGYLKPILIGVGVILLVILIFKIVSWIPTSSSNQ

SEQ ID NO: 11 - DNA, 2618 bp, Artificial Sequence
  LPHA1-gp47
atggcaccaccaatgacactgcaacaatggatcatttggaaaaaaatgaataaagcgcatgaggcacttcaaaatacaacaactgtgactgaacagcagaaggaacaaattatactggacattcaaaatgaagaagtacaaccaactaggagagataaatttagatatctgctttatacttgttgtgctactagctcaagagtattggcctggatgtttttagtttgtatattgttaatcattgttttggtttcatgctttgtgactatatccagaatacaatggaataaggatattcaggtattaggacctgtaatagactggaatgttactcaaagagctgtttatcaacccttacagactagaaggattgcacgtgatcagatttgcattggttaccatgcaaacaactcgacagagcaggttgacacaataatggaaaagaacgttactgttacacatgcccaagacatactggaaaagacacacaatgggaagctctgcgatctagatggagtgaagcctctaattttgagagattgtagtgtagctggatggctcctcggaaacccaatgtgtgacgaattcatcaatgtgccggaatggtcttacatagtggagaaggccaatccagtcaatgacctctgttacccaggggatttcaatgactatgaagaattgaaacacctattgagcagaataaaccattttgagaaaattcagatcatccccaaaagttcttggtccagtcatgaagcctcattgggggtgagctcagcatgtccataccagggaaagtcctcctttttcagaaatgtggtatggcttatcaaaaagaacagtacatacccaacaataaagaggagctacaataataccaaccaagaagatcttttggtactgtgggggattcaccatcctaatgatgcggcagagcagacaaagctctatcaaaacccaaccacctatatttccgttgggacatcaacactaaaccagagattggtaccaagaatagctactagatccaaagtaaacgggcaaagtggaaggatggagttcttctggacaattttaaaaccgaatgatgcaatcaacttcgagagtaatggaaatttcattgctccagaatatgcatacaaaattgtcaagaaaggggactcaacaattatgaaaagtgaattggaatatggtaactgcaacaccaagtgtcaaactccaatgggggcgataaactctagcatgccattccacaatatacaccctctcaccatcggggaatgccccaaatatgtgaaatcaaacagattagtccttgcgactgggctcagaaatacccctcaaaggaaaagaacaagtgttgataataactatgctaagttaaggtctatggggtatgcacttacaggagcagtgcaaaccttatctcaaatatcagatattaatgatgaaaacttacagcaaggaatatatttattaagggatcatgtaataaccttaatggaagctacattgcatgatatatctgttatggaaggaatgtttgctgtacaacatttgcatacacatttgaatcatttgaagacaatgcttctagaaagaagaatagactggacctatatgtctagtacttggctacaacaacaattacagaaatctgatgatgagatgaaagtaataaagagaattgctagaagtttggtatattatgttaaacaaacccatagttctcccacagctacagcctgggagattggattatattatgaattggttatacctaaacatatttacttgaataattggaatgttgtcaatataggtcacttagttaaatcagctggacaattgactcatgtaactatagctcatccttatgaaataatcaataaggaatgtgtagagactatatatctgcatcttgaggactgcacaagacaagattatgtcatatgtgatgtggtaaagatagtgcagccttgtggcaatagctcagacacgagtgattgtcctgtctgggctgaagctgtaaaagaaccatttgtgcaagtcaatcctctgaaaaacggaagttatctggttttggcaagttccacagactgtcagatcccaccatatgttcctagcatcgtgactgttaatgaaacaacgtcatgctttggactggactttaaaaggccactggttgcggaagaaagattgagctttgagccacgactgccaaatctacaactaagattaccacatttggttggaattattgcaaaaatcaaagggataaaaatagaagtcacatcctctggagaaagtataaaagagcagattgaaagagcaaaagctgagctccttcgactggacattcacgagggagatactcctgcctggatacaacagctagctgcagcaacaaaggacgtctggccagcagcagcttctgctctacaaggaattggtaactttttatctgggactgcccaaggaatatttggaactgcctttagtctcttgggatacttaaagcctatcctaataggagtaggggtcattctcttggttattcttatatttaagattgtatcatggattcctacgagcagcagcaatcagtaggcggccgc

SEQ ID NO: 12 - protein, 869 aa, Artificial Sequence
  LPHA1-gp47 fusion
MAPPMTLQQWIIWRRMNRAHEALQNTTTVTEQQREQIILDIQNEEVQPTRRDRFRYLLYTCCATSSRVLAWMFLVCILLIIVLVSCFVTISRIQWNKDIQVLGPVIDWNVTQRAVYQPLQTRRIARDQICIGYHANNSTEQVDTIMEKNVTVTHAQNILEKTHNGKLCDLDGVKPLILRDCSVAGWLLGNPMCDEFINVPEWSYIVEKANPANDLCYPGDFNDYEELKHLLSRINHFEKIQIIPKSSWSNHEASSGVSSACPYNGKSSFFRNVVWLIKKNSAYPTIKRSYNNTNQEDLLILWGIHHPNDAAEQTRLYQNPITYISVGTSTLNQRLVPKIATRSKVNGQSGRMEFFWTILKPNDAINFESNGNFIAPEYAYKIVKKGDSAIMKSELEYGNCNTKCQTPMGAINSSMPFHNIHPLTIGECPKYVKSNRLVLATGLRNTPQRKRTSVDNNYAKLRSMGYALTGAVQTLSQISDINDENLQQGIYLLRDHVITLMEATLHDISVMEGMFAVQHLHTHLNHLKTMLLERRIDWTYMSSTWLQQQLQKSDDEMKVIKRIARSLVYYVKQTHSSPTATAWEIGLYYELVIPKHIYLNNWNVVNIGHLVKSAGQLTHVTIAHPYEIINKECVETIYLHLEDCTRQDYVICDVVKIVQPCGNSSDTSDCPVWAEAVKEPFVQVNPLKNGSYLVLASSTDCQIPPYVPSIVTVNETTSCFGLDFKRPLVAEERLSFEPRLPNLQLRLPHLVGIIAKIKGIKIEVTSSGESIKEQIERAKAELLRLDIHEGDTPAWIQQLAAATKDVWPAAASALQGIGNFLSGTAQGIFGTAFSLLGYLKPILIGVGVILLVILIFKIVSWIPTSSSNQ

SEQ ID NO: 13 - DNA, 2238 bp, Artificial Sequence
  HA1-gp47deltaCD fusion
atggagaaaatagtgcttctttttgcaatagtcagtcttgttaaaagtgatcagatttgcattggttaccatgcaaacaactcgacagagcaggttgacacaataatggaaaagaacgttactgttacacatgcccaagacatactggaaaagacacacaatgggaagctctgcgatctagatggagtgaagcctctaattttgagagattgtagtgtagctggatggctcctcggaaacccaatgtgtgacgaattcatcaatgtgccggaatggtcttacatagtggagaaggccaatccagtcaatgacctctgttacccaggggatttcaatgactatgaagaattgaaacacctattgagcagaataaaccattttgagaaaattcagatcatccccaaaagttcttggtccagtcatgaagcctcattgggggtgagctcagcatgtccataccagggaaagtcctcctttttcagaaatgtggtatggcttatcaaaaagaacagtacatacccaacaataaagaggagctacaataataccaaccaagaagatcttttggtactgtgggggattcaccatcctaatgatgcggcagagcagacaaagctctatcaaaacccaaccacctatatttccgttgggacatcaacactaaaccagagattggtaccaagaatagctactagatccaaagtaaacgggcaaagtggaaggatggagttcttctggacaattttaaaaccgaatgatgcaatcaacttcgagagtaatggaaatttcattgctccagaatatgcatacaaaattgtcaagaaaggggactcaacaattatgaaaagtgaattggaatatggtaactgcaacaccaagtgtcaaactccaatgggggcgataaactctagcatgccattccacaatatacaccctctcaccatcggggaatgccccaaatatgtgaaatcaaacagattagtccttgcgactgggctcagaaatacccctcaaaggaaaagaacaagtgttgataataactatgctaagttaaggtctatggggtatgcacttacaggagcagtgcaaaccttatctcaaatatcagatattaatgatgaaaacttacagcaaggaatatatttattaagggatcatgtaataaccttaatggaagctacattgcatgatatatctgttatggaaggaatgtttgctgtacaacatttgcatacacatttgaatcatttgaagacaatgcttctagaaagaagaatagactggacctatatgtctagtacttggctacaacaacaattacagaaatctgatgatgagatgaaagtaataaagagaattgctagaagtttggtatattatgttaaacaaacccatagttctcccacagctacagcctgggagattggattatattatgaattggttatacctaaacatatttacttgaataattggaatgttgtcaatataggtcacttagttaaatcagctggacaattgactcatgtaactatagctcatccttatgaaataatcaataaggaatgtgtagagactatatatctgcatcttgaggactgcacaagacaagattatgtcatatgtgatgtggtaaagatagtgcagccttgtggcaatagctcagacacgagtgattgtcctgtctgggctgaagctgtaaaagaaccatttgtgcaagtcaatcctctgaaaaacggaagttatctggttttggcaagttccacagactgtcagatcccaccatatgttcctagcatcgtgactgttaatgaaacaacgtcatgctttggactggactttaaaaggccactggttgcggaagaaagattgagctttgagccacgactgccaaatctacaactaagattaccacatttggttggaattattgcaaaaatcaaagggataaaaatagaagtcacatcctctggagaaagtataaaagagcagattgaaagagcaaaagctgagctccttcgactggacattcacgagggagatactcctgcctggatacaacagctagctgcagcaacaaaggacgtctggccagcagcagcttctgctctacaaggaattggtaactttttatctgggactgcccaaggaatatttggaactgcctttagtctcttgggatacttaaagcctatcctaataggagtaggggtcattctcttggttattcttatattt

SEQ ID NO: 14 - protein, 746 aa, Artificial Sequence
  HA-gp47deltaCD fusion
MEKIVLLFAIVSLVKSDQICIGYHANNSTEQVDTIMEKNVTVTHAQDILEKTHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQGKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNTPQRKRTSVDNNYAKLRSMGYALTGAVQTLSQISDINDENLQQGIYLLRDHVITLMEATLHDISVMEGMFAVQHLHTHLNHLKTMLLERRIDWTYMSSTWLQQQLQKSDDEMKVIKRIARSLVYYVKQTHSSPTATAWEIGLYYELVIPKHIYLNNWNVVNIGHLVKSAGQLTHVTIAHPYEIINKECVETIYLHLEDCTRQDYVICDVVKIVQPCGNSSDTSDCPVWAEAVKEPFVQVNPLKNGSYLVLASSTDCQIPPYVPSIVTVNETTSCFGLDFKRPLVAEERLSFEPRLPNLQLRLPHLVGIIAKIKGIKIEVTSSGESIKEQIERAKAELLRLDIHEGDTPAWIQQLAAATKDVWPAAASALQGIGNFLSGTAQGIFGTAFSLLGYLKPILIGVGVILLVILIF

SEQ ID NO: 15 - DNA, 2277 bp, Artificial Sequence
  HA-gp47 Codon Optimised
atggagaagatcgtgctgctgttcgccatcgtgagcctggtgaagagcgaccagatctgcatcggctaccacgccaacaacagcaccgagcaggtggacaccatcatggagaagaacgtgaccgtgacccacgcccaggacatcctggagaagacccacaacggcaagctgtgcgacctggacggagtgaagccactgatcctgagggactgcagcgtggcaggatggctgctgggaaacccaatgtgcgacgagttcatcaacgtgcccgagtggagctacatcgtggagaaggccaaccccgtgaacgacctgtgctaccccggcgacttcaacgactacgaggagctgaagcacctgctgagccggatcaaccacttcgagaagatccagatcatcccaaagagcagctggagcagccacgaggcaagcctgggcgtgagcagcgcctgcccataccagggcaagagcagcttcttccggaacgtggtgtggctgatcaagaagaacagcacctaccccaccatcaagcggagctacaacaacaccaaccaggaggacctgctggtgctgtggggcatccaccaccccaacgacgccgccgagcagaccaagctgtaccagaatcccaccacctacatcagcgtgggcaccagcaccctgaaccagcggctggtgccaaggatcgcaacccggagcaaggtgaacggacagagcggccggatggagttcttctggaccatcctgaagcccaacgacgccatcaacttcgagagcaacggcaacttcatcgcccccgagtacgcctacaagatcgtgaagaagggcgacagcaccatcatgaagagcgagctggagtacggcaactgcaacaccaagtgccagacccctatgggagccatcaacagcagcatgcccttccacaacatccaccccctgaccatcggcgagtgccccaagtacgtgaagagcaacaggctggtgctggcaaccggactgaggaacacccctcagaggaagcggaccagcgtggacaacaactacgccaagctgcggagcatgggctacgccctgaccggcgccgtgcagaccctgagccagatcagcgacatcaacgacgagaacctgcagcagggcatctacctgctgcgggaccacgtgatcaccctgatggaggccaccctgcacgacatcagcgtgatggagggcatgttcgccgtgcagcacctgcacacccacctgaaccacctgaagaccatgctgctggagcggcggatcgactggacctacatgagcagcacctggctgcagcagcagctgcagaagagcgacgacgagatgaaagtgatcaagcggatcgcccggagcctggtgtactacgtgaagcagacccacagcagccccaccgccaccgcctgggagatcggcctgtactacgagctggtcatccccaagcacatctacctgaacaactggaacgtggtgaacatcggccacctggtgaagagcgccggccagctgacccacgtgaccatcgcccacccctacgagatcatcaacaaggagtgcgtggagaccatctacctgcacctggaggactgcacccggcaggactacgtgatctgcgacgtggtgaagatcgtgcagccatgcggaaacagcagcgacaccagcgactgccccgtgtgggcagaggcagtgaaggagcccttcgtgcaggtgaaccccctgaagaacggcagctacctggtgctggccagcagcaccgactgccagatccctccatacgtgcccagcatcgtgaccgtgaacgagaccaccagctgcttcggcctggacttcaagcggcccctggtggcagaggagcggctgagcttcgagccaaggctgccaaacctgcagctgaggctgccacacctggtgggaatcatcgccaagatcaagggcatcaagatcgaggtgaccagcagcggcgagagcatcaaggagcagatcgagagggcaaaggcagagctgctgaggctggacatccacgagggcgacaccccagcatggatccagcagctggctgccgcaaccaaggacgtgtggccagctgccgcaagcgccctgcagggaatcggaaacttcctgagcggaaccgcacagggaatcttcggaaccgccttcagcctgctgggctacctgaagcccatcctgatcggcgtgggcgtgatcctgctcgtgatcctgatcttcaagatcgtgagctggatccctacctcttccagcaaccag

SEQ ID NO: 16 - DNA, 2297 bp, Artificial Sequence
  HA-gp47 fusion insert
ctcgagaccatggagaaaatagtgcttctttttgcaatagtcagtcttgttaaaagtgatcagatttgcattggttaccatgcaaacaactcgacagagcaggttgacacaataatggaaaagaacgttactgttacacatgcccaagacatactggaaaagacacacaatgggaagctctgcgatctagatggagtgaagcctctaattttgagagattgtagtgtagctggatggctcctcggaaacccaatgtgtgacgaattcatcaatgtgccggaatggtcttacatagtggagaaggccaatccagtcaatgacctctgttacccaggggatttcaatgactatgaagaattgaaacacctattgagcagaataaaccattttgagaaaattcagatcatccccaaaagttcttggtccagtcatgaagcctcattgggggtgagctcagcatgtccataccagggaaagtcctcctttttcagaaatgtggtatggcttatcaaaaagaacagtacatacccaacaataaagaggagctacaataataccaaccaagaagatcttttggtactgtgggggattcaccatcctaatgatgcggcagagcagacaaagctctatcaaaacccaaccacctatatttccgttgggacatcaacactaaaccagagattggtaccaagaatagctactagatccaaagtaaacgggcaaagtggaaggatggagttcttctggacaattttaaaaccgaatgatgcaatcaacttcgagagtaatggaaatttcattgctccagaatatgcatacaaaattgtcaagaaaggggactcaacaattatgaaaagtgaattggaatatggtaactgcaacaccaagtgtcaaactccaatgggggcgataaactctagcatgccattccacaatatacaccctctcaccatcggggaatgccccaaatatgtgaaatcaaacagattagtccttgcgactgggctcagaaatacccctcaaaggaaaagaacaagtgttgataataactatgctaagttaaggtctatggggtatgcacttacaggagcagtgcaaaccttatctcaaatatcagatattaatgatgaaaacttacagcaaggaatatatttattaagggatcatgtaataaccttaatggaagctacattgcatgatatatctgttatggaaggaatgtttgctgtacaacatttgcatacacatttgaatcatttgaagacaatgcttctagaaagaagaatagactggacctatatgtctagtacttggctacaacaacaattacagaaatctgatgatgagatgaaagtaataaagagaattgctagaagtttggtatattatgttaaacaaacccatagttctcccacagctacagcctgggagattggattatattatgaattggttatacctaaacatatttacttgaataattggaatgttgtcaatataggtcacttagttaaatcagctggacaattgactcatgtaactatagctcatccttatgaaataatcaataaggaatgtgtagagactatatatctgcatcttgaggactgcacaagacaagattatgtcatatgtgatgtggtaaagatagtgcagccttgtggcaatagctcagacacgagtgattgtcctgtctgggctgaagctgtaaaagaaccatttgtgcaagtcaatcctctgaaaaacggaagttatctggttttggcaagttccacagactgtcagatcccaccatatgttcctagcatcgtgactgttaatgaaacaacgtcatgctttggactggactttaaaaggccactggttgcggaagaaagattgagctttgagccacgactgccaaatctacaactaagattaccacatttggttggaattattgcaaaaatcaaagggataaaaatagaagtcacatcctctggagaaagtataaaagagcagattgaaagagcaaaagctgagctccttcgactggacattcacgagggagatactcctgcctggatacaacagctagctgcagcaacaaaggacgtctggccagcagcagcttctgctctacaaggaattggtaactttttatctgggactgcccaaggaatatttggaactgcctttagtctcttgggatacttaaagcctatcctaataggagtaggggtcattctcttggttattcttatatttaagattgtatcatggattcctacgagcagcagcaatcagtaggcggccgc

SEQ ID NO: 17 - DNA, 406 bp, Artificial Sequence
  LPHA1-gp47 insert
ctcgagatggcaccaccaatgacactgcaacaatggatcatttggagaagaatgaatagagcgcatgaggcacttcaaaatacaacaactgtgactgaacagcagagggaacaaattatactggacattcaaaatgaagaagtacaaccaactaggagagatagatttagatatctgctttatacttgttgtgctactagctcaagagtattggcctggatgtttttagtttgtatattgttaatcattgttttggtttcatgctttgtgactatatccagaatacaatggaataaggatattcaggtattaggacctgtaatagactggaatgttactcaaagagctgtttatcaacccttacagactagaaggattgcacgtgatcagatttgcattggttacc

SEQ ID NO: 18 - DNA, 470 bp, Artificial Sequence
  gp47deltaCD insert
gctagctgcagcaacaaaggacgtctggccagcagcagcttctgctctacaaggaattggtaactttttatctgggactgcccaaggaatatttggaactgcctttagtctcttgggatacttaaagcctatcctaataggagtaggggtcattctcttggttattcttatattttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataaggatcgatccgggctggcgtaatagcgaagaggcggccgc

SEQ ID NO: 19 - DNA, 2601 bp, Human immunodeficiency virus type 1
atgagagtgaaggggatcaggaggaattatcagcactggtggggatggggcacgatgctccttgggttattaatgatctgtagtgctacagaaaaattgtgggtcacagtctattatggggtacctgtgtggaaagaagcaaccaccactctattttgtgcatcagatgccaaagcatatgatacagaggcacataatgtttgggccacacatgcctgtgtacccacagaccccaacccacaagaagtagaattggtaaatgtgacagaaaattttaacatgtggaaaaataacatggtagaacagatgcatgaggatataatcagtttatgggatcaaagcctaaagccatgcgtaaaattaaccccactctgtgttactttaaattgcactgatttgaggaatactactaataccaataatagtactgataataacaatagtaatagcgagggaacaataaagggaggagaaatgaaaaactgctctttcaatatcgccacaagcataggagataagatgcagaaagaatatgcacttctttataaacttgatatagaaccaatagataatgataataccagctataggttgataagttgtaatacctcagtcattacacaagcttgtccaaagatatcctttgagccaattcccatacactattgtgccccggctggttttgcgattctaaagtgtaacgataaaaagttcagtggaaaaggatcatgtaaaaatgtcagcacagtacaatgtacacatggaattaggccagtagtatcaactcaactgctgttaaatggcagtctagcagaagaagaggtagtaattagatctgagaatttcacaaacaatgctaaaaccataatagtacagctgagagaacctgtaaaaattaattgttcaagacccaacaacaatacaagaaaaagtatacctatgggaccagggagagcattttatacaacaggacaaataataggagatataagacaagcacattgtaatattagtaaaacaaattggactaacgctttaaaacaggtagttgaaaaactaggggaacaattcaacaagacaaaaatagtctttacgaactcctcaggaggggacccagaaattgtaacgcacagttttaattgtgcaggggaatttttctactgtaatacaacacaactgtttgatagtatttggaatagtgagaatggtacttggaatattactagggggttaaataacactggaagaaatgacacaatcacactcccatgcaggataaaacaaattataaacaggtggcaggaagtaggaaaagcaatgtatgcccctcccatcaaaggaaacattagctgttcatcaaatattacagggctgctattaacaagagatggtggtaaggatgataatagcagggacgggaacgagaccttcagacctggaggaggagatatgagggacaattggagaagtgaattatataaatataaagtagtaaaaattgaaccattaggagtagcacccaccaaggcaaagagaagagtggtgcaaagagaagaaagagcagtgggactaggagctatgttcattgggttcttgggagcagcaggaagcactatgggcgcagcgtcagtgacgctgacggtacaggccaggcaattattgtctggtatagtgcaacagcagagcaatttgctgagagctattgaggcgcaacagcatctgttgcaactcacagtctggggcatcaagcagctccaggcaagaatcctggctgtggaaagatacctaaaggatcaacagctcctagggatttggggttgctctggaaaactcatttgcaccactactgtgccttggaatgctagttggagtaagaataaaactctggaagctatttggaataacatgacctggatgcagtgggacaaagagattgacaattacacaagcttaatatactccttaattgaagaatcgcagatccaacaagaaaagaatgaacaagaattattggaattagataaatgggcaaatctgtggaattggtttgacatatcaaactggctgtggtatataaaaatattcataatgatagtaggaggcttggtaggtttaaggatagtttttgttgtactttctatagtgaatagagttaggcagggatactcaccattatcgtttcagacccgcctcccagccccgaggggacccgacaggcccgaagaaatcgaagaagaaggtggagacagagacagagacagatccgggctcttagtggatggattcttaacacttatctgggtcgacctgcggagcctgtgcctcttcagctaccaccgcttgagagacttactcttgattgtgacgaggattgtggaacttctgggacgcagggggtgggaaatcctcaaatattggtggaatctcctgcagtattggagtcaggaactaaagaatagtgctgttagcttgttcaacgccaccgccatagcagtagctgagggaacagatagggttatagaagtattacaaagagttggtagagctttgctccacatacctacaagaataagacagggcttggaaagggctttgctataa

SEQ ID NO: 20 - protein, 866 aa, Human immunodeficiency virus type 1
MRVKGIRRNYQHWWGWGTMLLGLLMICSATEKLWVTVYYGVPVWKEATTTLFCASDAKAYDTEAHNVWATHACVPTDPNPQEVELVNVTENFNMWKNNMVEQMHEDIISLWDQSLKPCVKLTPLCVTLNCTDLRNTTNTNNSTDNNNSNSEGTIKGGEMKNCSFNIATSIGDKMQKEYALLYKLDIEPIDNDNTSYRLISCNTSVITQACPKISFEPIPIHYCAPAGFAILKCNDKKFSGKGSCKNVSTVQCTHGIRPVVSTQLLLNGSLAEEEVVIRSENFTNNAKTIIVQLREPVKINCSRPNNNTRKSIPMGPGRAFYTTGQIIGDIRQAHCNISKTNWTNALKQVVEKLGEQFNKTKIVFTNSSGGDPEIVTHSFNCAGEFFYCNTTQLFDSIWNSENGTWNITRGLNNTGRNDTITLPCRIKQIINRWQEVGKAMYAPPIKGNISCSSNITGLLLTRDGGKDDNSRDGNETFRPGGGDMRDNWRSELYKYKVVKIEPLGVAPTKAKRRVVQREERAVGLGAMFIGFLGAAGSTMGAASVTLTVQARQLLSGIVQQQSNLLRAIEAQQHLLQLTVWGIKQLQARILAVERYLKDQQLLGIWGCSGKLICTTTVPWNASWSKNKTLEAIWNNMTWMQWDKEIDNYTSLIYSLIEESQIQQEKNEQELLELDKWANLWNWFDISNWLWYIKIFIMIVGGLVGLRIVFVVLSIVNRVRQGYSPLSFQTRLPAPRGPDRPEEIEEEGGDRDRDRSGLLVDGFLTLIWVDLRSLCLFSYHRLRDLLLIVTRIVELLGRRGWEILKYWWNLLQYWSQELKNSAVSLFNATAIAVAEGTDRVIEVLQRVGRALLHIPTRIRQGLERALL

SEQ ID NO: 21 - DNA, 2727 bp, Artificial Sequence
  HIV-1 R2 gp120-FP-gp47deltaCD Codon Optimised
atgcgggtgaagggcatccggcggaactaccagcactggtggggctggggcaccatgctgctgggcctgctgatgatctgcagcgccaccgagaagctgtgggtgaccgtgtactacggagtgcccgtgtggaaggaggcaaccaccaccctgttctgcgcaagcgacgcaaaggcatacgacaccgaggcacacaacgtgtgggcaacccacgcatgcgtgccaaccgacccaaacccacaggaggtggagctggtgaacgtgaccgagaacttcaacatgtggaagaacaacatggtggagcagatgcacgaggacatcatcagcctgtgggaccagagcctgaagccctgcgtgaagctgacccccctgtgcgtgaccctgaactgcaccgacctgcggaacaccaccaacaccaacaacagcaccgacaacaacaacagcaacagcgagggcaccatcaagggcggcgagatgaagaactgcagcttcaacatcgccaccagcatcggcgacaagatgcagaaggagtacgccctgctgtacaagctggacatcgagcccattgacaacgacaacaccagctaccggctgatcagctgcaacaccagcgtgatcacccaggcctgccccaagatcagcttcgagcccatccccatccactactgcgcccccgccggcttcgccatcctgaagtgcaacgacaagaagttcagcggcaagggcagctgcaagaacgtgagcaccgtgcagtgcacccacggaatcaggccagtggtgagcacccagctgctgctgaacggaagcctggcagaggaggaggtggtcatccggagcgagaacttcaccaacaacgccaagaccatcatcgtgcagctgcgggagcccgtgaagatcaactgcagccggcccaacaacaacacccggaaaagcatccccatgggaccaggaagggccttctacaccaccggacagatcatcggcgacatcaggcaggcacactgcaacatcagcaagaccaactggaccaacgccctgaagcaggtggtggagaagctgggcgagcagttcaacaagaccaagatcgtgttcaccaacagcagcggcggcgaccccgagatcgtgacccacagcttcaactgcgccggcgagttcttctactgcaacaccacccagctgttcgacagcatctggaacagcgagaacggcacctggaacatcacccggggcctgaacaacaccggccggaacgacaccatcaccctgccctgccggatcaagcagatcatcaaccggtggcaggaagtgggcaaggccatgtacgccccccccatcaagggcaacatcagctgcagcagcaacatcaccggactgctgctgacccgggacggaggcaaggacgacaacagccgggacggaaacgagaccttccggccaggaggaggcgacatgagggacaactggcggagcgagctgtacaagtacaaggtggtgaagatcgagccactgggagtggcaccaaccaaggcaaagaggagggtggtgcagagcgaggagagcgccgtgggcctgggcgccatgttcatcggcttcctgggagcagcaggaagcaccatgggagcagcaagcgtgaccctgaccgtgcaggcacggcagctgatggaggccaccctgcacgacatcagcgtgatggagggcatgttcgccgtgcagcacctgcacacccacctgaaccacctgaagaccatgctgctggagcggcggatcgactggacctacatgagcagcacctggctgcagcagcagctgcagaagagcgacgacgagatgaaagtgatcaagcggatcgcccggagcctggtgtactacgtgaagcagacccacagcagccccaccgccaccgcctgggagatcggcctgtactacgagctggtcatccccaagcacatctacctgaacaactggaacgtggtgaacatcggccacctggtgaagagcgccggccagctgacccacgtgaccatcgcccacccctacgagatcatcaacaaggagtgcgtggagaccatctacctgcacctggaggactgcacccggcaggactacgtgatctgcgacgtggtgaagatcgtgcagccatgcggaaacagcagcgacaccagcgactgccccgtgtgggcagaggcagtgaaggagcccttcgtgcaggtgaaccccctgaagaacggcagctacctggtgctggccagcagcaccgactgccagatccccccctacgtgcccagcatcgtgaccgtgaacgagaccaccagctgcttcggcctggacttcaagcggcccctggtggcagaggagcggctgagcttcgagccaaggctgccaaacctgcagctgaggctgccacacctggtgggaatcatcgccaagatcaagggcatcaagatcgaggtgaccagcagcggcgagagcatcaaggagcagatcgagagggcaaaggcagagctgctgaggctggacatccacgagggcgacaccccagcatggatccagcagctggctgccgcaaccaaggacgtgtggccagctgccgcaagcgccctgcagggaatcggaaacttcctgagcggaaccgcacagggaatcttcggaaccgccttcagcctgctgggctacctgaagcccatcctgatcggcgtgggcgtgatcctgctcgtgatcctgatcttc

SEQ ID NO: 22 - protein, 909 aa, Artificial Sequence
  HIV-1 R2 gp120-FP-gp47deltaCD
MRVKGIRRNYQHWWGWGTMLLGLLMICSATEKLWVTVYYGVPVWKEATTTLFCASDAKAYDTEAHNVWATHACVPTDPNPQEVELVNVTENFNMWKNNMVEQMHEDIISLWDQSLKPCVKLTPLCVTLNCTDLRNTTNTNNSTDNNNSNSEGTIKGGEMKNCSFNIATSIGDKMQKEYALLYKLDIEPIDNDNTSYRLISCNTSVITQACPKISFEPIPIHYCAPAGFAILKCNDKKFSGKGSCKNVSTVQCTHGIRPVVSTQLLLNGSLAEEEVVIRSENFTNNAKTIIVQLREPVKINCSRPNNNTRKSIPMGPGRAFYTTGQIIGDIRQAHCNISKTNWTNALKQVVEKLGEQFNKTKIVFTNSSGGDPEIVTHSFNCAGEFFYCNTTQLFDSIWNSENGTWNITRGLNNTGRNDTITLPCRIKQIINRWQEVGKAMYAPPIKGNISCSSNITGLLLTRDGGKDDNSRDGNETFRPGGGDMRDNWRSELYKYKVVKIEPLGVAPTKAKRRVVQSEESAVGLGAMFIGFLGAAGSTMGAASVTLTVQARQLMEATLHDISVMEGMFAVQHLHTHLNHLKTMLLERRIDWTYMSSTWLQQQLQKSDDEMKVIKRIARSLVYYVKQTHSSPTATAWEIGLYYELVIPKHIYLNNWNVVNIGHLVKSAGQLTHVTIAHPYEIINKECVETIYLHLEDCTRQDYVICDVVKIVQPCGNSSDTSDCPVWAEAVKEPFVQVNPLKNGSYLVLASSTDCQIPPYVPSIVTVNETTSCFGLDFKRPLVAEERLSFEPRLPNLQLRLPHLVGIIAKIKGIKIEVTSSGESIKEQIERAKAELLRLDIHEGDTPAWIQQLAAATKDVWPAAASALQGIGNFLSGTAQGIFGTAFSLLGYLKPILIGVGVILLVILIF

SEQ ID NO: 23 - DNA, 2747 bp, Artificial Sequence
  gp120-FP-gp47deltaCD insert
ctcgagaccatgcgggtgaagggcatccggcggaactaccagcactggtggggctggggcaccatgctgctgggcctgctgatgatctgcagcgccaccgagaagctgtgggtgaccgtgtactacggagtgcccgtgtggaaggaggcaaccaccaccctgttctgcgcaagcgacgcaaaggcatacgacaccgaggcacacaacgtgtgggcaacccacgcatgcgtgccaaccgacccaaacccacaggaggtggagctggtgaacgtgaccgagaacttcaacatgtggaagaacaacatggtggagcagatgcacgaggacatcatcagcctgtgggaccagagcctgaagccctgcgtgaagctgacccccctgtgcgtgaccctgaactgcaccgacctgcggaacaccaccaacaccaacaacagcaccgacaacaacaacagcaacagcgagggcaccatcaagggcggcgagatgaagaactgcagcttcaacatcgccaccagcatcggcgacaagatgcagaaggagtacgccctgctgtacaagctggacatcgagcccattgacaacgacaacaccagctaccggctgatcagctgcaacaccagcgtgatcacccaggcctgccccaagatcagcttcgagcccatccccatccactactgcgcccccgccggcttcgccatcctgaagtgcaacgacaagaagttcagcggcaagggcagctgcaagaacgtgagcaccgtgcagtgcacccacggaatcaggccagtggtgagcacccagctgctgctgaacggaagcctggcagaggaggaggtggtcatccggagcgagaacttcaccaacaacgccaagaccatcatcgtgcagctgcgggagcccgtgaagatcaactgcagccggcccaacaacaacacccggaaaagcatccccatgggaccaggaagggccttctacaccaccggacagatcatcggcgacatcaggcaggcacactgcaacatcagcaagaccaactggaccaacgccctgaagcaggtggtggagaagctgggcgagcagttcaacaagaccaagatcgtgttcaccaacagcagcggcggcgaccccgagatcgtgacccacagcttcaactgcgccggcgagttcttctactgcaacaccacccagctgttcgacagcatctggaacagcgagaacggcacctggaacatcacccggggcctgaacaacaccggccggaacgacaccatcaccctgccctgccggatcaagcagatcatcaaccggtggcaggaagtgggcaaggccatgtacgccccccccatcaagggcaacatcagctgcagcagcaacatcaccggactgctgctgacccgggacggaggcaaggacgacaacagccgggacggaaacgagaccttccggccaggaggaggcgacatgagggacaactggcggagcgagctgtacaagtacaaggtggtgaagatcgagccactgggagtggcaccaaccaaggcaaagaggagggtggtgcagagcgaggagagcgccgtgggcctgggcgccatgttcatcggcttcctgggagcagcaggaagcaccatgggagcagcaagcgtgaccctgaccgtgcaggcacggcagctgatggaggccaccctgcacgacatcagcgtgatggagggcatgttcgccgtgcagcacctgcacacccacctgaaccacctgaagaccatgctgctggagcggcggatcgactggacctacatgagcagcacctggctgcagcagcagctgcagaagagcgacgacgagatgaaagtgatcaagcggatcgcccggagcctggtgtactacgtgaagcagacccacagcagccccaccgccaccgcctgggagatcggcctgtactacgagctggtcatccccaagcacatctacctgaacaactggaacgtggtgaacatcggccacctggtgaagagcgccggccagctgacccacgtgaccatcgcccacccctacgagatcatcaacaaggagtgcgtggagaccatctacctgcacctggaggactgcacccggcaggactacgtgatctgcgacgtggtgaagatcgtgcagccatgcggaaacagcagcgacaccagcgactgccccgtgtgggcagaggcagtgaaggagcccttcgtgcaggtgaaccccctgaagaacggcagctacctggtgctggccagcagcaccgactgccagatccccccctacgtgcccagcatcgtgaccgtgaacgagaccaccagctgcttcggcctggacttcaagcggcccctggtggcagaggagcggctgagcttcgagccaaggctgccaaacctgcagctgaggctgccacacctggtgggaatcatcgccaagatcaagggcatcaagatcgaggtgaccagcagcggcgagagcatcaaggagcagatcgagagggcaaaggcagagctgctgaggctggacatccacgagggcgacaccccagcatggatccagcagctggctgccgcaaccaaggacgtgtggccagctgccgcaagcgccctgcagggaatcggaaacttcctgagcggaaccgcacagggaatcttcggaaccgccttcagcctgctgggctacctgaagcccatcctgatcggcgtgggcgtgatcctgctcgtgatcctgatcttctgagcggccgc

SEQ ID NO: 24 - protein, 74 aa, Human foamy virus
MEATLHDISVMEGMFAVQHLHTHLNHLKTMLLERRIDWTYMSSTWLQQQLQKSDDEMKVIKRIARSLVYYVKQT

SEQ ID NO: 25 - DNA, 2657 bp, Artificial Sequence
  gp120-FP-gp47  MSD/CD insert
ctcgagaccatgcgggtgaagggcatccggcggaactaccagcactggtggggctggggcaccatgctgctgggcctgctgatgatctgcagcgccaccgagaagctgtgggtgaccgtgtactacggagtgcccgtgtggaaggaggcaaccaccaccctgttctgcgcaagcgacgcaaaggcatacgacaccgaggcacacaacgtgtgggcaacccacgcatgcgtgccaaccgacccaaacccacaggaggtggagctggtgaacgtgaccgagaacttcaacatgtggaagaacaacatggtggagcagatgcacgaggacatcatcagcctgtgggaccagagcctgaagccctgcgtgaagctgacccccctgtgcgtgaccctgaactgcaccgacctgcggaacaccaccaacaccaacaacagcaccgacaacaacaacagcaacagcgagggcaccatcaagggcggcgagatgaagaactgcagcttcaacatcgccaccagcatcggcgacaagatgcagaaggagtacgccctgctgtacaagctggacatcgagcccattgacaacgacaacaccagctaccggctgatcagctgcaacaccagcgtgatcacccaggcctgccccaagatcagcttcgagcccatccccatccactactgcgcccccgccggcttcgccatcctgaagtgcaacgacaagaagttcagcggcaagggcagctgcaagaacgtgagcaccgtgcagtgcacccacggaatcaggccagtggtgagcacccagctgctgctgaacggaagcctggcagaggaggaggtggtcatccggagcgagaacttcaccaacaacgccaagaccatcatcgtgcagctgcgggagcccgtgaagatcaactgcagccggcccaacaacaacacccggaaaagcatccccatgggaccaggaagggccttctacaccaccggacagatcatcggcgacatcaggcaggcacactgcaacatcagcaagaccaactggaccaacgccctgaagcaggtggtggagaagctgggcgagcagttcaacaagaccaagatcgtgttcaccaacagcagcggcggcgaccccgagatcgtgacccacagcttcaactgcgccggcgagttcttctactgcaacaccacccagctgttcgacagcatctggaacagcgagaacggcacctggaacatcacccggggcctgaacaacaccggccggaacgacaccatcaccctgccctgccggatcaagcagatcatcaaccggtggcaggaagtgggcaaggccatgtacgccccccccatcaagggcaacatcagctgcagcagcaacatcaccggactgctgctgacccgggacggaggcaaggacgacaacagccgggacggaaacgagaccttccggccaggaggaggcgacatgagggacaactggcggagcgagctgtacaagtacaaggtggtgaagatcgagccactgggagtggcaccaaccaaggcaaagaggagggtggtgcagagcgaggagagcgccgtgggcctgggcgccatgttcatcggcttcctgggagcagcaggaagcaccatgggagcagcaagcgtgaccctgaccgtgcaggcacggcagctgatggaggccaccctgcacgacatcagcgtgatggagggcatgttcgccgtgcagcacctgcacacccacctgaaccacctgaagaccatgctgctggagcggcggatcgactggacctacatgagcagcacctggctgcagcagcagctgcagaagagcgacgacgagatgaaagtgatcaagcggatcgcccggagcctggtgtactacgtgaagcagacccacagcagccccaccgccaccgcctgggagatcggcctgtactacgagctggtcatccccaagcacatctacctgaacaactggaacgtggtgaacatcggccacctggtgaagagcgccggccagctgacccacgtgaccatcgcccacccctacgagatcatcaacaaggagtgcgtggagaccatctacctgcacctggaggactgcacccggcaggactacgtgatctgcgacgtggtgaagatcgtgcagccatgcggaaacagcagcgacaccagcgactgccccgtgtgggcagaggcagtgaaggagcccttcgtgcaggtgaaccccctgaagaacggcagctacctggtgctggccagcagcaccgactgccagatccccccctacgtgcccagcatcgtgaccgtgaacgagaccaccagctgcttcggcctggacttcaagcggcccctggtggcagaggagcggctgagcttcgagccaaggctgccaaacctgcagctgaggctgccacacctggtgggaatcatcgccaagatcaagggcatcaagatcgaggtgaccagcagcggcgagagcatcaaggagcagatcgagagggcaaaggcagagctgctgaggctggacatccacgagggcgacaccccagcatggatccagcagctggctgccgcaaccaaggacgtgtggccagctgccgcaagcgccctgcagggaatcggaaacttcctgagcggaaccgcatagtgagcggccgc

SEQ ID NO: 26 - protein, 880 aa, Artificial Sequence
  gp120-FP-gp47 TMD/CD
CDMRVKGIRRNYQHWWGWGTMLLGLLMICSATEKLWVTVYYGVPVWKEATTTLFCASDAKAYDTEAHNVWATHACVPTDPNPQEVELVNVTENFNMWKNNMVEQMHEDIISLWDQSLKPCVKLTPLCVTLNCTDLRNTTNTNNSTDNNNSNSEGTIKGGEMKNCSFNIATSIGDKMQKEYALLYKLDIEPIDNDNTSYRLISCNTSVITQACPKISFEPIPIHYCAPAGFAILKCNDKKFSGKGSCKNVSTVQCTHGIRPVVSTQLLLNGSLAEEEVVIRSENFTNNAKTIIVQLREPVKINCSRPNNNTRKSIPMGPGRAFYTTGQIIGDIRQAHCNISKTNWTNALKQVVEKLGEQFNKTKIVFTNSSGGDPEIVTHSFNCAGEFFYCNTTQLFDSIWNSENGTWNITRGLNNTGRNDTITLPCRIKQIINRWQEVGKAMYAPPIKGNISCSSNITGLLLTRDGGKDDNSRDGNETFRPGGGDMRDNWRSELYKYKVVKIEPLGVAPTKAKRRVVQSEESAVGLGAMFIGFLGAAGSTMGAASVTLTVQARQLMEATLHDISVMEGMFAVQHLHTHLNHLKTMLLERRIDWTYMSSTWLQQQLQKSDDEMKVIKRIARSLVYYVKQTHSSPTATAWEIGLYYELVIPKHIYLNNWNVVNIGHLVKSAGQLTHVTIAHPYEIINKECVETIYLHLEDCTRQDYVICDVVKIVQPCGNSSDTSDCPVWAEAVKEPFVQVNPLKNGSYLVLASSTDCQIPPYVPSIVTVNETTSCFGLDFKRPLVAEERLSFEPRLPNLQLRLPHLVGIIAKIKGIKIEVTSSGESIKEQIERAKAELLRLDIHEGDTPAWIQQLAAATKDVWPAAASALQGIGNFLSGTA

SEQ ID NO: 27 - DNA, 1346 bp, Artificial Sequence
  HASigHAtag-gp47 CO gene
ctcgagaccatggagaagatcgtgctgctgttcgccatcgtgagcctggtgaagagctacccatacgatgttccagattacgctagcgtggacaacaactacgccaagctgcggagcatgggctacgccctgaccggcgccgtgcagaccctgagccagatcagcgacatcaacgacgagaacctgcagcagggcatctacctgctgcgggaccacgtgatcaccctgatggaggccaccctgcacgacatcagcgtgatggagggcatgttcgccgtgcagcacctgcacacccacctgaaccacctgaagaccatgctgctggagcggcggatcgactggacctacatgagcagcacctggctgcagcagcagctgcagaagagcgacgacgagatgaaagtgatcaagcggatcgcccggagcctggtgtactacgtgaagcagacccacagcagccccaccgccaccgcctgggagatcggcctgtactacgagctggtcatccccaagcacatctacctgaacaactggaacgtggtgaacatcggccacctggtgaagagcgccggccagctgacccacgtgaccatcgcccacccctacgagatcatcaacaaggagtgcgtggagaccatctacctgcacctggaggactgcacccggcaggactacgtgatctgcgacgtggtgaagatcgtgcagccatgcggaaacagcagcgacaccagcgactgccccgtgtgggcagaggcagtgaaggagcccttcgtgcaggtgaaccccctgaagaacggcagctacctggtgctggccagcagcaccgactgccagatccctccatacgtgcccagcatcgtgaccgtgaacgagaccaccagctgcttcggcctggacttcaagcggcccctggtggcagaggagcggctgagcttcgagccaaggctgccaaacctgcagctgaggctgccacacctggtgggaatcatcgccaagatcaagggcatcaagatcgaggtgaccagcagcggcgagagcatcaaggagcagatcgagagggcaaaggcagagctgctgaggctggacatccacgagggcgacaccccagcatggatccagcagctggctgccgcaaccaaggacgtgtggccagctgccgcaagcgccctgcagggaatcggaaacttcctgagcggaaccgcacagggaatcttcggaaccgccttcagcctgctgggctacctgaagcccatcctgatcggcgtgggcgtgatcctgctcgtgatcctgatcttcaagatcgtgagctggatccctacctcttccagcaaccagtgagcggccgc

SEQ ID NO: 28 - protein, 442 aa, Artificial Sequence
  HAsigHAtag-gp47
MEKIVLLFAIVSLVKSYPYDVPDYASVDNNYAKLRSMGYALTGAVQTLSQISDINDENLQQGIYLLRDHVITLMEATLHDISVMEGMFAVQHLHTHLNHLKTMLLERRIDWTYMSSTWLQQQLQKSDDEMKVIKRIARSLVYYVKQTHSSPTATAWEIGLYYELVIPKHIYLNNWNVVNIGHLVKSAGQLTHVTIAHPYEIINKECVETIYLHLEDCTRQDYVICDVVKIVQPCGNSSDTSDCPVWAEAVKEPFVQVNPLKNGSYLVLASSTDCQIPPYVPSIVTVNETTSCFGLDFKRPLVAEERLSFEPRLPNLQLRLPHLVGIIAKIKGIKIEVTSSGESIKEQIERAKAELLRLDIHEGDTPAWIQQLAAATKDVWPAAASALQGIGNFLSGTAQGIFGTAFSLLGYLKPILIGVGVILLVILIFKIVSWIPTSSSNQ

SEQ ID NO: 29 - protein, 44 aa, Artificial Sequence
  HIV-1 TM
SEIYTLIEESQNQQEKNEQELLELDKWASLWNWFDITKWLWYIK

SEQ ID NO: 30 - protein, 417 aa, Human foamy virus
SVDNNYAKLRSMGYALTGAVQTLSQISDINDENLQQGIYLLRDHVITLMEATLHDISVMEGMFAVQHLHTHLNHLKTMLLERRIDWTYMSSTWLQQQLQKSDDEMKVIKRIARSLVYYVKQTHSSPTATAWEIGLYYELVIPKHIYLNNWNVVNIGHLVKSAGQLTHVTIAHPYEIINKECVETIYLHLEDCTRQDYVICDVVKIVQPCGNSSDTSDCPVWAEAVKEPFVQVNPLKNGSYLVLASSTDCQIPPYVPSIVTVNETTSCFGLDFKRPLVAEERLSFEPRLPNLQLRLPHLVGIIAKIKGIKIEVTSSGESIKEQIERAKAELLRLDIHEGDTPAWIQQLAAATKDVWPAAASALQGIGNFLSGTAQGIFGTAFSLLGYLKPILIGVGVILLVILIFKIVSWIPTKKKNQ

SEQ ID NO: 31 - protein, 48 aa, Human foamy virus
SVDNNYAKLRSMGYALTGAVQTLSQISDINDENLQQGIYLLRDHVITL

SEQ ID NO: 32 - protein, 174 aa, Human foamy virus
HSSPTATAWEIGLYYELVIPKHIYLNNWNVVNIGHLVKSAGQLTHVTIAHPYEIINKECVETIYLHLEDCTRQDYVICDVVKIVQPCGNSSDTSDCPVWAEAVKEPFVQVNPLKNGSYLVLASSTDCQIPPYVPSIVTVNETTSCFGLDFKRPLVAEERLSFEPRLPNLQLRLP

SEQ ID NO: 33 - protein, 77 aa, Human foamy virus
HLVGIIAKIKGIKIEVTSSGESIKEQIERAKAELLRLDIHEGDTPAWIQQLAAATKDVWPAAASALQGIGNFLSGTA

SEQ ID NO: 34 - protein, 28 aa, Human foamy virus
FGTAFSLLGYLKPILIGVGVILLVILIF

SEQ ID NO: 35 - protein, 13 aa, Human foamy virus
KIVSWIPTKKKNQ

SEQ ID NO: 36 - protein, 325 aa, Human foamy virus
MEATLHDISVMEGMFAVQHLHTHLNHLKTMLLERRIDWTYMSSTWLQQQLQKSDDEMKVIKRIARSLVYYVKQTHSSPTATAWEIGLYYELVIPKHIYLNNWNVVNIGHLVKSAGQLTHVTIAHPYEIINKECVETIYLHLEDCTRQDYVICDVVKIVQPCGNSSDTSDCPVWAEAVKEPFVQVNPLKNGSYLVLASSTDCQIPPYVPSIVTVNETTSCFGLDFKRPLVAEERLSFEPRLPNLQLRLPHLVGIIAKIKGIKIEVTSSGESIKEQIERAKAELLRLDIHEGDTPAWIQQLAAATKDVWPAAASALQGIGNFLSGTA

SEQ ID NO: 37 - protein, 248 aa, Human foamy virus
MEATLHDISVMEGMFAVQHLHTHLNHLKTMLLERRIDWTYMSSTWLQQQLQKSDDEMKVIKRIARSLVYYVKQTHSSPTATAWEIGLYYELVIPKHIYLNNWNVVNIGHLVKSAGQLTHVTIAHPYEIINKECVETIYLHLEDCTRQDYVICDVVKIVQPCGNSSDTSDCPVWAEAVKEPFVQVNPLKNGSYLVLASSTDCQIPPYVPSIVTVNETTSCFGLDFKRPLVAEERLSFEPRLPNLQLRLP

(19) United States
(12) Patent Application Publication
(10) Pub. No.: US 2012/0164155 A1
(43) Pub. Date: Jun. 28, 2012

(54) CHIMERIC MOLECULES

(75) Inventor: Elizabeth Grgacic, Melbourne (AU)

(73) Assignee: The Macfarlane Burnet Institute for Medical Research and Public Health Ltd., Melbourne, Victoria (AU)

(21) Appl. No.: 13/380,272

(22) PCT Filed: Jun. 22, 2010
(86) PCT No.: PCT/AU2010/000775

§ 371 Date: Dec. 22, 2011

Related U.S. Application Data
(60) Provisional application No. 61/219,216, filed on Jun. 22, 2009.

(51) Int. Cl. A61K 39/21; C07H 21/04; G01N 33/566; A61P 31/14; C07K 16/10; C07K 19/00; A61P 37/04

(52) U.S. Cl. 424/159.1; 530/350; 536/23.4; 424/192.1; 530/389.4; 436/501; 435/7.92

(57) ABSTRACT

The invention relates to chimeric proteins comprising an antigen and a trimer forming portion or a trimer and virus-like particle forming portion of foamy virus envelope protein (FV TM). The trimer or trimer and virus-like particle forming portion comprises i) full length foamy virus transmembrane protein; ii) foamy virus transmembrane protein absent a functional cytoplasmic domain; iii) foamy virus transmembrane protein absent a functional cytoplasmic domain and transmembrane domain; iv) foamy virus ectodomain comprising N-terminal heptad repeat region and cysteine rich region between N-terminal heptad repeat region and C-terminal α-helical region; v) N-terminal heptad repeat region; vi) a functional variant of any one of i) to v); or vii) any one of i) to vi) lacking an FV fusion peptide domain. In particular, the antigen is an antigen of a virus envelope protein, such as HIV gp 120. Soluble and membrane bound forms of trimeric and higher oligomeric forms of the chimeric proteins are provided as well as nucleic acid molecules encoding and expressing same, viral-like particles comprising same, compositions including pharmaceutical compositions, host cells and kits. Methods are described for producing immune responses including antibodies determined by the chimeric protein or VLP, as well as methods of screening using the chimeric protein, VLP and/or antibodies.

### FIELD

The present invention relates generally to complex chimeric proteins and in some embodiments to chimeric viral envelope proteins, and methods for making same. In an illustrative embodiment, the invention provides stable soluble and membrane binding forms of trimeric chimeric HIV gp120 and viral-like particles (VLPs) comprising membrane binding forms thereof. In one aspect, the present invention provides inter alia soluble or particle compositions suitable for eliciting or screening immune responses to viral infections.

### BACKGROUND

Bibliographic details of references in the subject specification are also listed at the end of the specification.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Nucleotide and amino acid sequences are referred to by a sequence identifier number (SEQ ID NO:). The SEQ ID NOs: correspond numerically to the sequence identifiers <400>1 (SEQ ID NO:1), <400>2 (SEQ ID NO:2), etc. A summary of sequence identifiers is provided in Table 1. A sequence listing is provided after the claims.

A major hurdle to the development of viral vaccines such as HIV and influenza vaccines, is the selection of an immunogen that is capable of eliciting a broadly neutralising antibody response. In the case of HIV, several rational design strategies based on the envelope glycoproteins that mimic the native conformation of mature HIV envelope trimers on the virion surface have been intensely studied. These include envelope modifications that promote trimerisation, such as SHIV/HIV chimeras and modifications that stabilise the gp120-gp41 association (SOSgp140) or the heptad repeat (reviewed by Hedestam et al., Nature Reviews Microbiology 6: 143-155, 2008). More recently the use of the envelope strain, R2, from an isolate capable of CD4-independent infection and able to elicit broadly neutralising cross-reactive antibodies when expressed as soluble gp140_{R2} has proved to be effective (Zhang et al., PNAS 104(24): 10193-10198, 2007). However, soluble gp140 (gp120-gp41ectodomain) exhibits a heterogeneity not observed with the virion-associated envelope. The propensity to produce varied oligomeric forms appears to reside in the labile nature of the association of the N terminal heptad repeat of gp41 molecules.

Vaccine-induced immune responses to viruses ideally produce neutralising antibodies and effective T-cells against antigens that are present on invading viruses or against virus antigens presented on the surface of host cells. There is a need in the art for methods of producing subunit vaccine compositions that, at least, more closely resemble aspects of their in vivo counterparts.

### SUMMARY

Each embodiment in this specification is to be applied mutatis mutandis to every other embodiment unless expressly stated otherwise.

In one embodiment, the present invention provides a chimeric protein comprising a heterologous (non-FV) polypeptide or peptide of interest and at least a trimer forming or a trimer and VLP forming portion of a transmembrane protein of foamy virus envelope protein (FV TM).

In an illustrative embodiment, the polypeptide or peptide of interest is a pathogenic viral envelope polypeptide or peptide.

In a particular embodiment, the trimer or trimer and VLP forming portion of FV TM comprises:
i) full length foamy virus transmembrane protein;
ii) foamy virus transmembrane protein absent a functional cytoplasmic domain;
iii) foamy virus transmembrane protein absent a functional cytoplasmic domain and transmembrane domain;
iv) foamy virus ectodomain comprising N-terminal heptad repeat region and cysteine rich region between N-terminal heptad repeat region and C-terminal α-helical region;
v) N-terminal heptad repeat region;
vi) a functional variant of any one of i) to v) optionally having conservative substitutions therein; or
vii) any one of i) to vi) lacking an FV fusion peptide domain.

Viral-like particles comprising membrane binding forms of the chimeric proteins are provided as well as nucleic acid molecules encoding the chimeric proteins.

Compositions comprising the antibodies, or antibody binding fragments, proteins, VLPs and encoding nucleic acids are contemplated as well as pharmaceutical compositions comprising same and methods of treatment or prophylaxis using same.

A large number of different methods are contemplated using the chimeric trimeric proteins of the present invention. These include methods for producing neutralising antibodies to the subject POI, methods for screening antibodies for antibodies that bind specifically to trimeric or other oligomeric forms of POI produced using the subject chimeric proteins.

Kits comprising the subject proteins, VLPs, nucleic acids, host cells or antibodies or antigen binding fragments are also provided.

The above summary is not and should not be seen in any way as an exhaustive recitation of all embodiments of the present invention.

### BRIEF DESCRIPTION OF THE FIGURES

Some figures contain colour representations or entities. Coloured versions of the figures are available from the patentee upon request or from an appropriate Patent Office. A fee may be imposed if obtained from a Patent Office.

FIG. 1 is a diagrammatic representation of Foamy Virus and HIV-1 envelope protein: [symbol] transmembrane domain [symbol] N-terminal heptad repeat; [symbol] C-terminal α-helical region; F fusion peptide; c conserved cysteine residues; C═C intramolecular disulphide bond. Alignment of N terminal heptad repeat sequence with a and d positions in grey and underlined.

FIG. 2 is a diagrammatic representation of the SU and TM of various POI in comparison with that of FV: F N-terminal fusion peptide; [symbol] Heptad repeat; [symbol] Membrane-spanning domain.

FIG. 3 is a schematic representation of influenza HA1-gp47 fusions: [symbol] Heptad repeat; [symbol] Membrane-spanning domain.

FIG. 4 is a photographic representation illustrating immunofluorescent staining of 293T cells transfected with: A. pHA1-gp47 B. pHA1-gp47ΔCD and C. mock transfection. Cell stained with monoclonal antibody to HA (MAb 149).

FIG. 5 is a photographic representation illustrating expression of LPHA1-gp47 in the presence or absence of 5 uM of the proteosome inhibitor, lactacystin.

FIGS. 6 A and B are graphical representations illustrating sucrose gradient profile of HA1-gp47(A) and HA1-gp47ΔCD (B) VLPs. Fractions detected by ELISA using a conformational anti-HA monoclonal antibody, MAb 149.

FIG. 7 is a photographic representation of a Western blot of membrane fraction and VLPs probed with anti-HA MAb 8D2 (Hytest).

FIG. 8 is a graphical representation illustrating a 20-50% OptiPrep gradient profile of HA1-gp47 Codon Optimised showing that intracellular VLPs are produced but not secreted from the cell.

FIGS. 9 A and B are a photographic representations illustrating A) chemical cross-linking of HA1-gp47 soluble membrane protein and VLPs. Trimers of the expected size (approximately 255 kDa) were detected in membrane and VLP populations; and B) trypsin digestion of HA1-gp47 indicating the correct conformation of globular HA1 bound or fused to gp47.

FIG. 10 is a graphical representation illustrating a longitudinal anti-HA response after boosting with HA-gp47 VLPs.

FIG. 11 is a graphical representation illustrating anti-HA endpoint titres for mice immunised with HA-gp47 VLPs.

FIG. 12 is a schematic representation of HIV-1 gp120-FP-gp47 fusion. HIV gp120/gp41 sequence in grey; F N-terminal fusion peptide of HIV-1 gp41; [symbol] Heptad repeat; [symbol] Membrane-spanning domain.

FIG. 13 is a photographic representation illustrating immunofluorescent staining of 293T cells transfected with pCI gp120-FP-gp47ΔCD or mock transfection. Cells stained with monoclonal antibody to gp120 showing that the fusion construct is expressed and reacts with a neutralising monoclonal antibody (MAb 2G12).

FIG. 14 is a photographic representation illustrating Western blot of soluble membrane protein, probed with HIV plasma IgG showing that the gp120-FP-gp47ΔCD fusion protein has a molecular mass of approximately 140-180 kDa.

FIG. 15 is a graphical representation of a 20-50% OptiPrep gradient of gp120-FP-gp47ΔCD showing both intracellular and secreted VLPs are detected by reactivity with HIV-1 plasma IgG.

FIGS. 16 A and B are graphical representations of a OptiPrep 20-50% 16 hour gradient of VLPs. Fractions detected by ELISA using HIV plasma IgG, MAb b12 or MAb 2G12. A) intracellular VLPs; and B) secreted VLPs.

FIG. 17 is a photographic representation showing the glycosylation pattern of secreted and intracellular VLPs comprising gp120-FP-gp47ΔCD.

FIG. 18 are representations of HFV nucleotide and amino acid sequences; the nucleotide sequence for HFV gp47 (SEQ ID NO: 2); the amino acid sequence of HFV gp47 (SEQ ID NO: 3); the nucleotide sequence of HFV leader peptide of gp47 (SEQ ID NO: 4); the amino acid sequence of HFV leader peptide subunit of gp47 (SEQ ID NO: 5); and the amino acid sequence of HFV gp47 with a deleted cytoplasmic domain (SEQ ID NO: 6).

FIG. 19 provides a nucleotide and amino acid sequence for HA1-gp47 fusions and sequences for influenza HA1-gp47 fusions (SEQ ID NOs: 7 to 17).

FIG. 20 provides a nucleotide sequence of the HA-gp47 fusion insert (Xhol 5′ restriction site and 3′ NotI site are in bold; ATG is underlined with the Kozac sequence in italics) for HA-gp47 fusions in the Examples.

FIG. 21 provides a plasmid map of HA-gp47 constructs. pCI HA-gp47→Graphic Map. HA1-gp47 fusion start Xhol 1091 end Notl 3380.

FIG. 22 provides a plasmid map of HA-gp47 constructs. pCI LPHA1-gp47→Graphic Map. LPHA1-gp47 start Xhol 1091 end Notl 3707.

FIG. 23 provides a plasmid map of HA-gp47 constructs. pCI HA-gp47ΔCD→Graphic Map. HA1-gp47ΔCD start Xhol 1091 end Notl 3625.

FIG. 24 provides plasmid map of HA-gp47 constructs. pCI HA1-gp47 Codon Opt.→Graphic Map. HA1-gp47 CO start Xhol 1091 end Notl 13380.

FIG. 25 provides nucleotide and amino acid sequences of HIV-1 R2 envelope proteins; sequences and codon optimised nucleotide sequences and amino acid sequence 1 of HIV-1 R2gp120-FP-gp47ΔCD.

FIG. 26 provides the nucleotide sequence of gp120-FP-gp47ΔCD insert referred to in the Examples. (Xhol 5′ restriction site and 3′ NotI site are in bold; ATG and stop codon is underlined with a minimum Kozac sequence in italics) Codon Optimised.

FIG. 27 provides a construct plasmid map for pCI R2gp120-FP-gp47ΔCD→Graphic Map. gp120-FP-gp47ΔCd fusion starts Xhol 1091 end Notl 3830.

FIG. 28 is a representation of a Western blot showing trimer formation of soluble gp120-FP-gp47ΔCD on VLPs by chemical crosslinking. Chemical cross-linking of purified soluble trimer gp140AD8 and gp120-gp47 VLPs with 0.04; 0.2 and 1 mM EGS; recomb. gp140 AD8 was purified by size exclusion chromatography to trimers * indicates some cleavage of gp140 to gp120 still occurs.

FIG. 29 is a representation of electron microscopy images confirming VLP formation of intracellular and secreated VLPs. A. EM negative staining of gp120-gp47VLPs: A-C secreted VLPs; D-E intracellular VLPs; F serum-derived duck hepatitis B virus subviral particles (Klingmüller and Schaller, J Viral. 67(12): 7414-22, 1993). VLPs are 50-70 nm in diameter. EM performed by K. Goldie University of Melbourne Bio21 EM Unit. B. EM negative staining of gp120-gp47 Intracellular VLPs. EM performed by J. Mackenzie LaTrobe University.

FIG. 30 is a graphical representation of ELISA results showing the antibody response (seroconversion) of rabbits to secreted and intracellular VLPs (gp120-FP-gp47ΔCD) tested against gp140R2.

FIG. 31 is a graphical representation of results of endpoint titrations (see Example 9).

FIG. 32 is a graphical representation of results showing antibody responses of rabbits to soluble trimer, gp140R2 and soIHIFV.

FIG. 33 provides an illustration of soluble trimer constructs including chimeric trimers gp120-FP-gp47 (soIHIFV).

FIG. 34 is a representation of nucleotide and amino acid sequences of gp120-FP-gp47Δ MSD/CD insert for soluble HIFV trimer 5′ Xho and 3′ NotI restriction sites are in bold and ATG and stop codons are underlined. The amino acid sequence of gp120-FP-gp47Δ TMD/CD gp47 sequence is in bold.

FIG. 35 is a plasmid map for HIV-1R2 gp120-FP-gp47ΔTMD/CD.

FIG. 36 is a representation of results of trimer formation of soluble HIFV by chemical cross-linking with 1 mM EGS. Soluble HIV-1 trimer gp140 (AD8 uncleaved version).

FIG. 37 is a graphical representation of results showing binding of soluble trimers to neutralising human monoclonal antibodies b12 and 2G12.

FIG. 38 is a representation of a Blue Native PAGE-Western blot showing soluble HIFV present as trimer and higher order oligomers with little or no monomer or dimer species evident.

FIG. 39 is a representation of a Blue Native PAGE-Western blot showing that soluble HIFV trimers are more stable than gp14OR2.

FIG. 40 is representation of nucleotide and amino acid sequences of HASigHAtag-gp47CO.

FIG. 41 is a plasmid map of HASigHAtag-gp47CO.

FIG. 42 illustrates structure, size and expression of HAtag-gp47. A. Schematic diagram of construct for expression of FV gp47 with N terminal HA tag. B. Western blot of HA-gp47 expression in 293T cells, soluble membrane fraction. C. Immunofluorescence staining with monoclonal anti-HA tag. A. 293T cells transfected with pCI HA-gp47 B. Mock transfected cells.

FIG. 43 illustrates VLP formation of HAtag-gp47. A. Western blot of intracellular VLPs (I) and secreted VLPs (S) after sedimentation through 20% sucrose onto a 70% sucrose cushion. Soluble membrane protein (M). Bands detected with Mab HA-tag. B. Western blot of gradient fractions of HA-gp47 and gp120-FP-gp47 VLPs after sedimentation through a 20-50% OptiPrep gradient.

FIG. 44 is a schematic diagram of Foamy Virus TM protein (gp47) showing major structural regions: FP—fusion peptide region; N-terminal heptad repeat; c conserved cysteine residues; C-terminal α-helical domain; membrane spanning domain shown as black box, numbers refer to amino acid length of designated regions. Based on secondary structure predictions of Wang and Mulligan, J. Gen. Virol. 80: 245-254, 1999.

FIG. 45 is an amino acid sequence of HFV gp47 with heptad repeat and predicted α-helical domains underlined, conserved cysteine residues are in bold and membrane spanning domain (MSD) in bold italics. Amino acid numbers for key residues and start and end of heptad repeat and MSD are indicated above the sequence. The predicted furin cleavage site is indicated by the arrow.

FIG. 46 is an alignment of Foamy Virus gp47 amino acid sequences from Human, Simian, Bovine, Equine and Feline Foamy Virus. The N-terminal heptad region is underlined.

FIG. 47 is a diagrammatic representation of A. HIV-1 TM protein gp41: ▪ transmembrane domain [symbol] N-terminal heptad repeat; [symbol] C-terminal heptad repeat; F fusion peptide; C═C intramolecular disulphide bond. The sequence for the conserved membrane proximal region, extending to the C terminal heptad repeat to encompass the epitopes of the neutralising monoclonal antibodies 2F5 and 4E10 (underlined) is shown. The a and d positions of the C-terminal heptad repeats are indicated above the sequence. Amino acid numbering corresponds to its position in gp160 of HIV-1 HXB2; and B. FV TM protein gp47 indicating potential substitutions of gp47 sequences in the α-helical domain/MPR with the MPR of HIV-1 (italics). The predicted (SOSUIcoil) a and d heptad repeat positions in the FV gp-47 α-helical domain sequence are indicated below the sequence. Substitutions may encompass various lengths of the core α-helical and HIV-1 MPR sequences as indicated by the dotted lines. ▪ transmembrane domain [symbol] N-terminal heptad repeat; [symbol] C-terminal α-helical region.

### BRIEF DESCRIPTION OF THE TABLES

Table 1 provides a description of the SEQ ID NOs provided herein.

Table 2 provides a list of suitable naturally occurring proteogenic amino acids.

Table 3 provides an amino acid sub-classification.

Table 4 provides exemplary amino acid substitutions.

Table 5 provides a list of abbreviations.

### DETAILED DISCUSSION OF PARTICULAR EMBODIMENTS

The articles “a” and “an” are used herein to refer to one or to more than one (i.e. to at least one) of the grammatical object of the article. By way of example, “a cell” means one cell or more than one cell. A “POI” means one POI or more than one POI.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Abbreviations are set out in Table 5.

The present invention provides inter alia methods for engineering proteins to facilitate the production of oligomeric proteins of interest (POI), such as trimeric virus envelope proteins that are bound to and exploit the structural advantages of Foamy Virus (FV) envelope protein. It is proposed, in some embodiments, that the chimeric trimeric proteins produced in this way exhibit improved homogeneity, stability, or half-life, or are improved substrates for neutralizing, conformational or other forms of antibody production.

Foamy Virus (FV), also known as Spumavirus is a non-pathogenic retrovirus of primates, cattle and cats. Simian Foamy Viruses have been isolated from chimpanzees, New and Old World monkeys and apes. Humans are susceptible to cross infection from primates and a Human Foamy Virus isolated from an individual in Kenya was shown to be related to Simian Foamy Virus (SFV). Human Foamy Virus is known under the other names, SFVcpz(hu) and Prototype Foamy Virus (Lui et al., PLoS Pathogens 4(7): 1-22, 2008).

Unlike other retroviruses, such as HIV-1, the Foamy Virus envelope proteins are able to assemble without other viral structural proteins into subviral particles and these bud from the endoplasmic reticulum membrane, much like hepatitis B viruses and sub-viral particles of hepatitis B viruses (Shaw et al., J. Virol. 77(4): 2338-48, 2003).

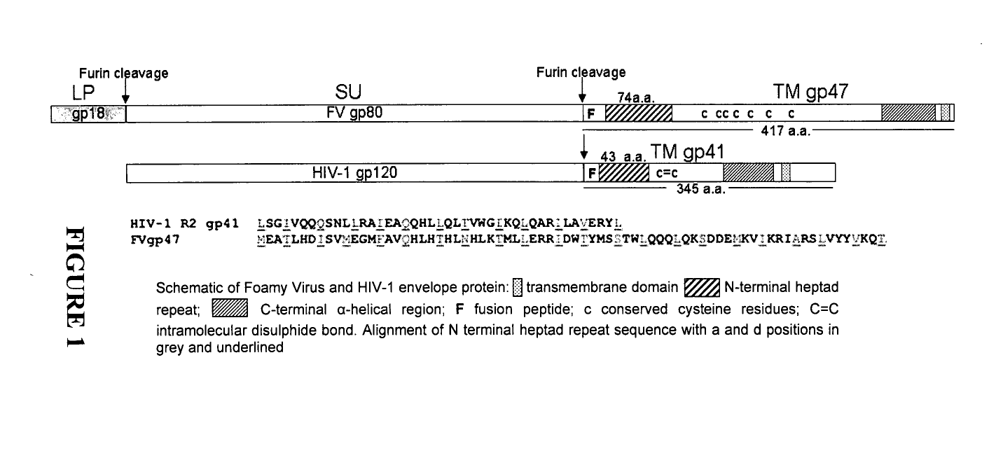

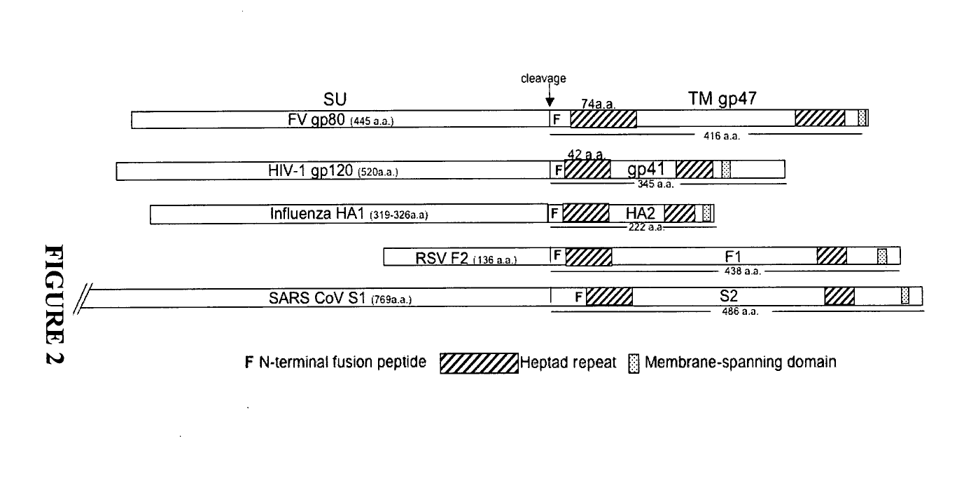

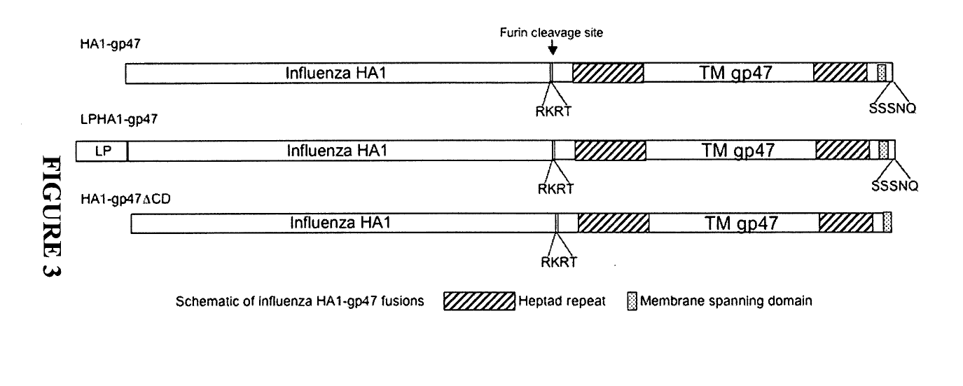

In nature, the FV envelope protein is produced in the cell as a polyprotein which is processed by the cellular enzyme, furin, into three subunits: leader peptide (LP), surface (SU) and transmembrane (TM). The LP, SU and TM protein subunits are all assembled into particles. It is assumed that FV SU and TM are associated non-covalently in particles like other retroviruses. Consistent with other retroviruses, the envelope protein forms trimers with the FV gp47 containing a similar arrangement of the N terminal fusion peptide, N and C terminal heptad repeats with intervening cysteine residues external to the transmembrane domain (as shown in FIGS. 1 and 33).

The N-terminal heptad repeat is known as the trimer interaction domain, facilitating assembly of three envelope monomers. The “heptad repeat” is a repeating structural motif of seven amino acids consisting generally of a first hydrophobic amino acid (H) followed by two polar (P) amino acids followed by a single hydrophobic (H) amino acid followed by three polar (P) amino acids. Each consecutive position of the motif (HPPHPPP) is referred to as a, b, c, d, e, f and g, respectively.

Electron microscopy of Human Foamy Virus (HFV) shows the envelope forms distinct trimers in hexameric arrays on the particle surface with no monomer visible, in contrast to HIV-1 cryoEM tomography analysis where there is no pattern of distribution of trimers, there are low numbers of trimers and they are heterogeneous (Wilk et al., J. Virol. 74(6): 2885-2887, 2000 incorporated herein in its entirety by reference). The clarity of HFV trimers implies that they are more stable. It has been proposed that the greater stability may be attributed to the longer heptad repeat domain of gp47 (74 amino acids compared with HIV-1 gp47 heptad repeat of 43 amino acids).

The subject invention is not limited to particular screening procedures for agents, specific formulations of agents and various medical methodologies, as such may vary. The terminology used herein is for the purpose of describing particular embodiments only and is not intended to be limiting.

Unless defined otherwise, all technical and scientific terms used herein have the same meanings as commonly understood by one of ordinary skill in the art to which this invention belongs. Any materials and methods similar or equivalent to those described herein can be used to practise or test the present invention. Practitioners are particularly directed to Ream et al., eds., Molecular Biology Techniques: An Intensive Laboratory Course, Academic Press, 1998; Newton and Graham eds., PCR, Introduction to Biotechniques Series, 2nd ed., Springer Verlag, 1997; Sambrook et al., Molecular Cloning: A Laboratory Manual, Cold Spring Harbor Laboratory, Cold Spring Harbor, N.Y., 1989, Coligan et al., Current Protocols in Protein Science, John Wiley & Sons, Inc. 1995-1997, in particular Chapters 1, 5 and 6, and Ausubel et al., Cell Immunol., 193(1): 99-107, 1999; Colowick and Kaplan, eds., Methods In Enzymology, Academic Press, Inc.; Weir and Blackwell, eds., Handbook of Experimental Immunology, Vols. I-IV, Blackwell Scientific Publications, 1986; Joklik ed., Virology, 3rd Edition, 1988; Fields and Knipe, eds, Fundamental Virology, 2nd Edition, 1991; Fields et al., eds, Virology, 3rd Edition, Lippincott-Raven, Philadelphia, Pa., 1996.

The present invention provides a chimeric protein comprising a heterologous (non-FV) polypeptide or peptide of interest and at least a trimer forming or a trimer and virus-like particle (VLP) forming portion of a transmembrane protein of foamy virus envelope protein (FV TM).

Various chimeric trimeric and higher oligomeric proteins comprising FV TM and a POI have been produced using HIV gp120 or influenza virus HAI envelope proteins as illustrative POI. For example, a trimer forming portion of FV TM comprising the N-terminal heptad repeat domain, C-terminal α-helical portion and intervening cysteine rich region of FV gp47 was produced as a fusion protein with HIV-1 gp120 and fusion peptide from HIV-1 gp41 and formed stable soluble HIV-1 gp120-FV TM trimers of the correct size with little or no monomer species. In another example, a trimer forming portion of FV TM comprising the N-terminal heptad repeat domain, C-terminal α-helical portion, intervening cysteine rich region, and a membrane spanning domain of FV TM was produced as a fusion protein with HIV-1 gp120 and formed VLPs comprising chimeric trimeric proteins.

FV TM is able to form trimers unaccompanied by other viral envelope proteins and accordingly, in some embodiments, chimeric FV TM proteins are contemplated wherein the POI is a polypeptide or peptide of a viral or non-viral pathogen. In some embodiments, the POI is an antigenic polypeptide or peptide of a non-viral pathogen or tumour antigen or autoimmune antigen. The POI may be positioned within the linear amino acid sequence of the FV TM, or it may be at one or both ends of an FV TM including the trimer and/or VLP forming portions described herein.

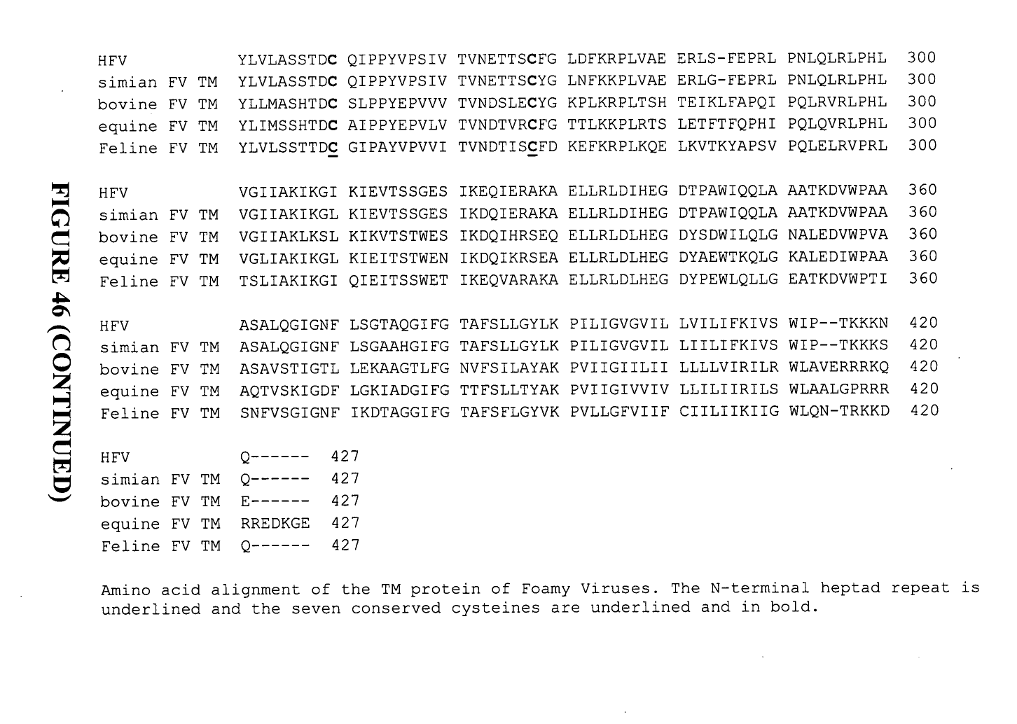

The term “FV TM” or “foamy virus transmembrane protein” is used through out to refer to a full length protein having an amino acid sequence which is the same or similar to one found in nature. The term also includes functional portions of the full length protein, referred to as trimer or trimer and VLP forming portions of the full length protein. The difference between the trimer forming portion and the trimer and VLP forming portion resides in the presence of a membrane spanning domain for efficient VLP formation. The term further includes functional variants that are trimer or trimer and VLP forming variants of the full length or trimer forming portions. Human foamy virus glycoprotein TM gp47, as well as trimer and trimer and VLP forming portions of human foamy virus TM gp47, provide illustrative examples of the invention. Further illustrative examples of FV TM include simian, feline, bovine and equine foamy virus transmembrane proteins as set out in FIG. 46 showing highly conserved amino acid sequences including within the ectodomain and N-terminal heptad repeat regions. The term FV TM is used to encompass all functionally analogous homologs, including orthologs and paralogs, isoforms and variants in any species.

The foamy virus may be from any species, and be any subspecies or type, although primate, simian (SFV) or human (HFV) forms are particularly contemplated. In accordance with the present invention, FV TM encompasses any naturally-occurring form from any animal species as well as their biologically active portions and variants or derivatives of these, as defined herein.

The amino acid sequence of N-terminal heptad repeat regions from other (non-FV) viruses are quite diverse. The HIV heptad repeat is about 43 amino acids, that of SARs S1 is reported as having 122 amino acids. RSV, Newcastle Disease virus and Measles virus heptad repeat regions are reported as approximately 43 amino acids. Influenza A, B, and C viruses are reported to have a dispersed heptad repeat arrangement of 16 amino acids followed by 20 amino acids that do not form the seven amino acid repeat pattern followed by 25 amino acids which follow the repeat patterns.

By “chimeric” is meant that the protein comprises sequences derived from at least two different organisms, such as different species, sub-species or variety/clades. The components of the chimera may be attached to each other by covalent or non-covalent bonds.

In an illustrative embodiment, chimeric proteins are produced wherein at least two chimeric components derived from different viral species are linked by covalent bonds, either by being expressed as part of the same expression product or by direct synthesis. In both cases the chimeric protein is referred to as a fusion protein.

For the avoidance of doubt, the term heterologous is used to convey that the polypeptide or peptide of interest, including the viral envelope protein or a peptide of interest is not derived from foamy virus. Foamy virus is a non-pathogenic organism while the viral POI of the invention are derived from pathogenic viruses.

By “derived from” is meant naturally occurring forms and functional variants of naturally occurring forms and therefore includes sequences directly or indirectly derived from an organism. Generally, a viral polypeptide is “derived from” a particular polypeptide of a virus (viral polypeptide) if it is (i) encoded by an open reading frame of a polynucleotide of that virus (viral polynucleotide), or (ii) displays sequence similarity to polypeptides of that virus as described herein.

The phrase “a heterologous (non-FV) polypeptide or peptide of interest” or “POI” is, in some embodiments, replaced by the term “an antigen”. Antigens are particularly antigens of pathogens or conditions against which an immune response is sought.

The term “polypeptide/s of interest” or “POI” includes a polypeptide, a peptide, an epitope or a series of peptides or epitopes which is/are selected for presentation, in an assay or to a subject, as trimers in a trimeric FV TM and/or as trimers in a trimeric FV TM within a VLP structure. The polypeptide of interest may be positioned at one or both ends of FV TM and/or within the linear amino acid sequence of an FV TM. In some embodiments, higher order oligomeric forms including trimer-dimers, tetramers etc are also provided.

In some embodiments, the POI is an antigenic polypeptide of a pathogen or one or more antigenic (immunogenic) portions thereof. One form of POI is a viral envelope protein not derived from foamy virus. One non-limiting aspect of the present invention is illustrated using viral envelope polypeptides which form trimers at least to some extent in their native state.

In some embodiments, the trimeric structure of the POI within the chimeric trimer or oligomer or as a VLP more closely resembles a native configuration and, in some particular embodiments, provides a more stable and/or more uniform form thereof.

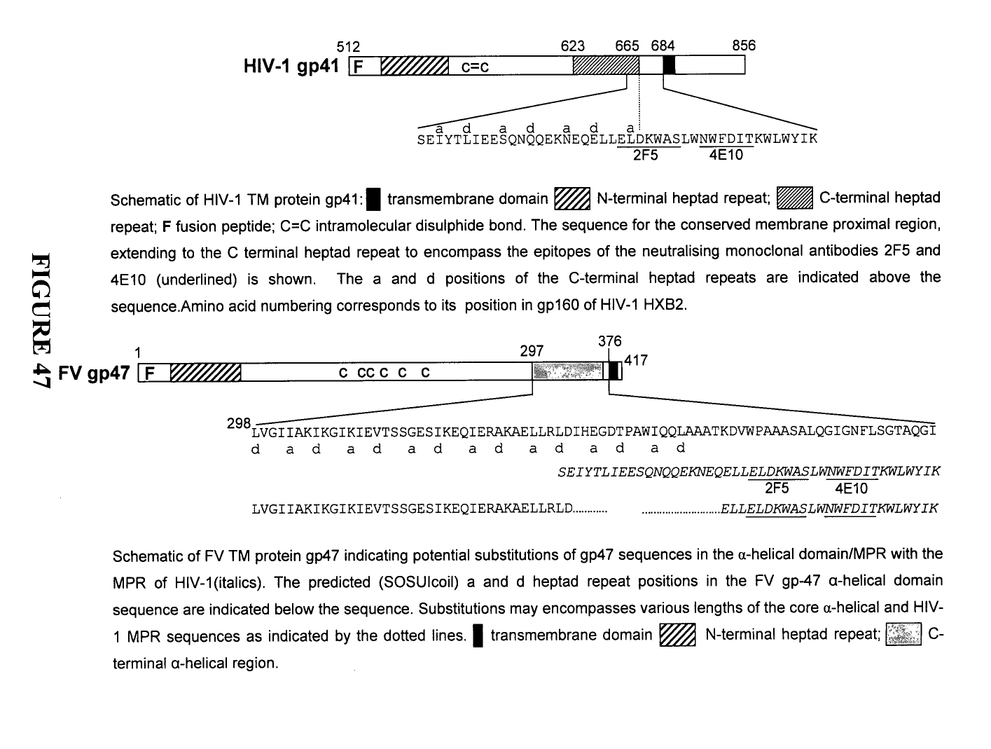

FV TM described herein forms soluble trimers and VLPs in the absence of a viral surface envelope polypeptide. The FV TM is therefore useful alone as a soluble or membrane binding trimer (or higher oligomeric form) or VLP. The FV TM trimeric proteins may include a POI. In some embodiments, the FV TM includes a terminal addition of a POI or is modified to comprise a POI. In one illustrative example, the FV TM comprises as a POI an antigenic peptide of a pathogen. An illustrative example is the membrane proximal region (MPR) of HIV-1. The MPR peptide may be introduced in a homologous region of FV TM such as between the C-terminal heptad region and membrane spanning domain, as illustrated in FIG. 47. In another example, the FP of FVTM may be substituted for the FP of HIV. Thus, it is proposed that peptides comprising epitopes of pathogens such as HIV are locked into a stable conformation in association with FV TM and are likely to engender and/or bind specific binding agents including neutralising antibodies.

In a particular embodiment, a trimeric FV TM protein is produced without a POI as defined above, although a detectable tag may be included. In this example, the detectable tag may be considered as a POI to facilitate detection of the FV TM protein.

The invention is illustrated by reference to non-FV viral envelope proteins which form trimers by interaction with the FV TM. However, the invention is not limited to viral envelope proteins as POI nor to trimers and extends to any protein that forms dimeric, trimeric, tetrameric or other oligomeric forms via binding to the TM protein of FV envelope protein. Non-envelope viral proteins are expressly contemplated as POI in the present chimeric polypeptides. Non-viral proteins include transcription factors, such as heat shock transcription factor, vitamin B2 receptor, macrophage scavenger protein and to other dimeric, tetrameric or oligomeric protein whose three-dimensional or other structure can be stabilised using a FV TM including at least the N-terminal heptad repeat domain therefrom. Further, non-viral pathogens or disease or autoimmune antigens are contemplated, including antigenic polypeptides and peptides from bacteria, parasites, fungi, and cancers, as known by those skilled in the art.

The terms “polypeptide” “protein” and “peptide” and “glycoprotein” are used interchangeably and mean a polymer of amino acids not limited to any particular length. The term does not exclude modifications such as myristylation, glycosylation, phosphorylation and addition or deletion of signal sequences.

The term “virus-like particle” or “VLP” is used in a broad sense to mean a particle or three dimensional structure which, like sub-viral particles of enveloped viruses, form particles by self assembly or folding of envelope proteins within a lipid bilayer. VLPs may be assembled in vitro or in vivo using techniques known in the art. VLPs are non-replicating and non-infectious. The presence of VLPs can be detected using conventional techniques known in the art, such as by electron microscopy, biophysical characterization, and the like. See, e.g., Baker et al., Biophys. J., 60: 1445-1456, 1991; Hagensee et al., J. Virol., 68: 4503-4505, 1994. For example, VLPs can be isolated by density gradient centrifugation and/or identified by characteristic density banding. Alternatively, cryoelectron microscopy can be performed on vitrified aqueous VLP samples.

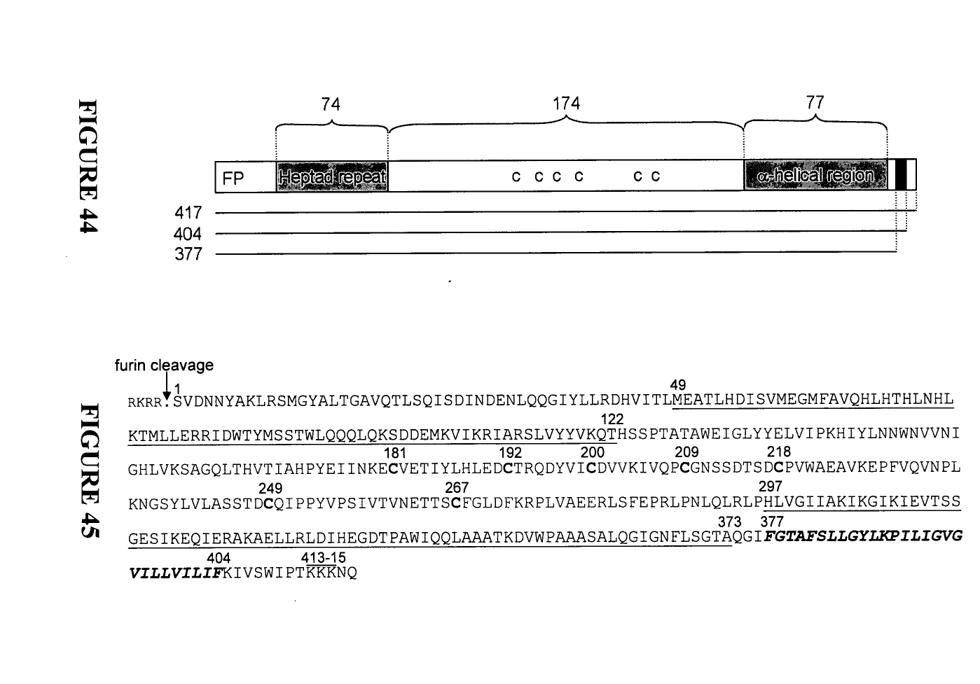

A “part” or “portion” or “region” or “domain” of a polypeptide such as a FV TM or a POI is defined as having a minimal size of at least about 3 amino acids or about 10 to 100 amino acids or about 70 to 180 amino acids or about 50 to 400 amino acids. This definition includes all sizes in the range of 3 to 400 amino acids including 50, 100, 200, 300 and 350 amino acids or molecules having any number of amino acids 3 and 400 amino acids. The gp47 polypeptide of HFV (HFV TM) comprises various portions including an N-terminal fusion peptide (approximately 48 amino acids) and a C-terminal cytoplasmic domain (about 13 amino acids). The gp47 further comprises an N-terminal heptad repeat region (about 74 amino acids), a C-terminal heptad repeat region also referred to as a C-terminal α-helical domain (about 77 amino acids) and an intervening cysteine-rich region comprising seven conserved cysteines (about 174 amino acids). At the C-terminal end between the C-terminal α-helical domain and the cytoplasmic domain resides a membrane spanning (transmembrane) domain (approximately 27 amino acids) (FIG. 44).

In some embodiments, the polypeptide or peptide of interest is a viral envelope polypeptide or peptide.

Any viral envelope protein may be engineered using the methods described in this specification. Reference herein to a non-FV virus includes without limitation a surface protein envelope protein from a virus from any virus family. Non-limiting examples of viral families include Adenoviridae, African swine fever-like viruses, Arenaviridae, Arterivirus, Astroviridae, Baculoviridae, Birnaviridae, Bunyaviridae, Caliciviridae, Circoviridae, Coronaviridae, Deltavirus, Filoviridae, Flaviviridae, Hepadnaviridae, Hepeviridae, Herpesviridae, Orthomyxoviridae, Paramyxoviridae, Picornaviridae, Poxyviridae, Reoviridae, Retroviridae and Rhabdoviridae. Particular virus envelope proteins are class I viral fusion proteins from Paramyxoviridae, Retroviridae and Filoviridae.

Illustrative antigens include those selected from influenza virus haemagglutinin, human respiratory syncytial virus G glycoprotein, core protein, matrix protein or other protein of Dengue virus, measles virus haemagglutinin, herpes simplex virus type 2 glycoprotein gB, poliovirus I VP1, envelope or capsid glycoproteins of HIV-I or HIV-II, hepatitis B surface antigen, diptheria toxin, streptococcus 24M epitope, gonococcal pilin, pseudorabies virus g50 (gpD), pseudorabies virus II (gpB), pseudorabies virusgIII (gpC), pseudorabies virus glycoprotein H, pseudorabies virus glycoprotein E, transmissible gastroenteritis glycoprotein 195, transmissible gastroenteritis matrix protein, swine rotavirus glycoprotein 38, swine parvovirus capsid protein, Serpulinahydrodysenteriae protective antigen, bovine viral diarrhea glycoprotein 55, newcastle disease virus hemagglutinin-neuraminidase, swine flu hemagglutinin, swine flu neuraminidase, foot and mouth disease virus, hog colera virus, swine influenza virus, African swine fever virus, mycoplasma liyopneutiioniae, infectious bovine rhinotracheitis virus, infectious bovine rhinotracheitis virus glycoprotein E, glycoprotein G, infectious laryngotracheitis virus, infectious laryngotracheitis virus glycoprotein G or glycoprotein I, a glycoprotein of La Crosse virus, neonatal calf diarrhoea virus, Venezuelan equine encephalomyelitis virus, punta toro virus, murine leukemia virus, mouse mammary tumor virus, hepatitis B virus core protein and hepatitis B virus surface antigen or a fragment or derivative thereof, antigen of equine influenza virus or equine herpes virus, including equine influenza virus type A/Alaska 91 neuraminidase, equine influenza virus typeA/Miami 63 neuraminidase, equine influenza virus type A/Kentucky 81 neuraminidase equine herpes virus type 1 glycoprotein B, and equine herpes virus type 1 glycoprotein D, antigen of bovine respiratory syncytial virus or bovine parainfluenza virus, bovine respiratory syncytial virus attachment protein (BRSV G), bovine respiratory syncytial virus fusion protein (BRSV F), bovine respiratory syncytial virus nucleocapsid protein (BRSVN), bovine parainfluenza virus type 3 fusion protein, bovine parainfluenza virus type 3 hemagglutinin neuraminidase, bovine viral diarrhoea virus glycoprotein 48 and glycoprotein 53.

Illustrative cancer antigens include KS ¼ pan-carcinoma antigen, ovarian carcinoma antigen (CA125), prostatic acid phosphate, prostate specific antigen, melanoma-associated antigen p97, melanoma antigen gp75, high molecular weight melanoma antigen (HMW-MAA), prostate specific membrane antigen, carcinoembryonic antigen (CEA), polymorphic epithelial mucin antigen, human milk fat globule antigen, colorectal tumor-associated antigens, CEA, TAG-72, LEA, Burkitt's lymphoma antigen-38.13, CD19, human B-lymphoma antigen-CD20, CD33, melanoma specific antigens, ganglioside GD2, ganglioside GD3, ganglioside GM2, ganglioside GM3, tumor-specific transplantation type of cell-surface antigen (TSTA), virally-induced tumor antigens, T-antigen DNA tumor viruses, Envelope antigens of RNA tumor viruses, oncofetal antigen-alpha-fetoprotein, CEA of colon, bladder tumor oncofetal antigen, differentiation antigen, human lung carcinoma antigen L6, L20, antigens of fibrosarcoma, human leukemia T cell antigen-Gp37, neoglycoprotein, sphingolipids, breast cancer antigen, EGFR (Epidermal growth factor receptor), HER2 antigen, polymorphic epithelial mucin, malignant human lymphocyte antigen-APO-1, differentiation antigen, including I antigen found in fetal erythrocytes, primary endoderm, I antigen found in adult erythrocytes, preimplantation embryos, I (Ma) found in gastric adenocarcinomas, M18, M39 found in breast epithelium, SSEA-1 found in myeloid cells, VEP8, VEP9, Myl, VIM-D5, Du56-22 found in colorectal cancer, TRA-1-85 (blood group H), C14 found in colonic adenocarcinoma, F3 found in lung adenocarcinoma, AH6 found in gastric cancer, Y hapten, LeY found in embryonal carcinoma cells, TL5 (blood group A), EGF receptor found in A431 cells, E1 series (blood group B) found in pancreatic cancer, FC10. 2 found in embryonal carcinoma cells, gastric adenocarcinoma antigen, CO-514 (blood group Lea) found in Adenocarcinoma, NS-10 found in adenocarcinomas, CO-43 (blood groupLeb), G49 found in EGF receptor of A431 cells, MH2 (blood groupALeb/Ley) found in colonic adenocarcinoma, 19.9 found in colon cancer, gastric cancer mucins, TsA7 found in myeloid cells, R24 found in melanoma, 4.2, GD3, D1.1, OFA-1, GM2, OFA-2, GD2, and M1:22:25:8 found in embryonal carcinoma cells, and SSEA-3 and SSEA-4 found in 4 to 8-cell stage embryos.

Of particular relevance as a non-FV virus in some aspects of the invention is a pathogenic virus. Non-limiting examples of pathogenic viruses are influenza haemagglutinin (HA); a lentivirus, such as HIV-1 glycoprotein (gp) 120 including the R2 subtype or HIV-2 gp125; a coronavirus, such as SARS Si glycoprotein; a paramyxovirus, such as respiratory syncytial virus (RSV) F2; or a flavivirus, such as Dengue virus E protein. As described further, the protein of any pathogen or disease may be combined with FV TM in accordance with the various aspects of the present invention.

One important group of pathogens is the primary systemic fungal pathogens of man such Coccidioides immitis, Histoplasma capsulatum, Blastomyces dermatitidis, and Paracoccidioides brasiliensis. Important opportunistic fungal pathogens which tend to rely upon an immunocompromised host include Cryptococcus neoformans, Pneumocystis jiroveci, Candida spp., Aspergillus spp., Penicillium marneffei, and Zygomycetes, Trichosporon beigelii, and Fusarium spp. A range of pathogenic fungi are associated with immunocompromised subjects including those with AIDS, with chemotherapy induced neutropenia or patients undergoing haematopoietic stem cell transplantation, among others.

In some embodiments, the pathogen is a microbe including a bacterium, fungus, virus, algae, parasite, (including ecto-or endo-parasites) prion, oomycetes, slime, moulds, nematode, mycoplasma and the like. By way of non-limiting example, the microbe is selected from one or more of the following orders, genera or species: Acinetobacter, Actinobacillus, Actinomycetes, Actinomyces, Aeromonas, Bacillus, Bacteroides, Bordetella, Borrelia, Brucella, Burkholderia, Campylobacter, Citrobacter, Clostridium, Corynebacterium, Enterobacter, Enterococcus, Erysipelothrix, Escherichia, Francisella, Haemophilus, Helicobacter, Klebsiella, Legionella, Leptospira, Listeria, Micrococcus, Moraxella, Morganella, Mycobacterium (tuberculosis), Nocardia, Neisseria, Pasteurella, Plesiomonas, Propionibacterium, Proteus, Providencia, Pseudomonas, Rhodococcus, Salmonella, Serratia, Shigella, Staphylococcus, Stenotrophomonas, Streptococcus, Treponema, Vibrio (cholera) and Yersinia (plague), Adenoviridae, African swine fever-like viruses, Arenaviridae (such as viral haemorrhagic fevers, Lassa fever), Astroviridae (astroviruses) Bunyaviridae (La Crosse), Calicivirid (Norovirus), Coronaviridae (Corona virus), Filoviridae (such as Ebola virus, Marburg virus), Parvoviridae (B19 virus), Flaviviridae (such as hepatitis C virus, Dengue viruses), Hepadnaviridae (such as hepatitis B virus, Deltavirus), Herpesviridae (herpes simplex virus, varicella zoster virus), Orthomyxoviridae (influenza virus) Papovaviridae (papilloma virus) Paramyxoviridae (such as human parainfluenza viruses, mumps virus, measles virus, human respiratory syncytial virus) Picornaviridae (common cold virus), Poxyiridae (small pox virus, orf virus, monkey poxvirus) Reoviridae (rotavirus) Retroviridae (human immunodeficiency virus) Paroviridae (paroviruses) Papillomaviridae, (papillomaviruses) alphaviruses and Rhabdoviridae (rabies virus), Trypanosoma, Leishmania, Giardia, Trichomonas, Entamoeba, Naegleria, Acanthamoeba, Plasmodium, Toxoplasma, Cryptosporidium, Isospora, Balantidium, Schistosoma, Echinostoma, Fasciolopsis, Clonorchis, Fasciola, Opisthorchis and Paragonimus, Pseudophyllidea (e.g., Diphyllobothrium) and Cyclophyllidea (e.g., Taenia). Pathogenic nematodes include species from the orders; Rhabditida (e.g., Strongyloides), Strongylida (e.g., Ancylostoma), Ascarida (e.g., Ascaris, Toxocara), Spirurida (e.g., Dracunculus, Brugia, Onchocerca, Wucheria), and Adenophorea (e.g., Trichuris and Trichinella), Prototheca and Ptiesteria, Absidia, Aspergillus, Blastomyces, Candida (yeast), Cladophialophera, Coccidioides, Cryptococcus, Cunninghamella, Fusarium, Histoplasma, Madurella, Malassezia, Microsporum, Mucor, Paecilomyces, Paracoccidioides, Penicillium, Pneumocystis, Pseudallescheria, Rhizopus, Rhodotorula, Scedosporium, Sporothrix, Trichophyton and Trichosporon. For the avoidance of doubt the pathogen may include an emerging or re-emerging pathogen or an organism which has never previously been identified as a pathogen in a particular subject.

In particular embodiments, the polypeptide or peptide is “of interest” as being derived from a pathogen or antigen of a subject in need of treatment or prophylaxis and comprising epitopes proposed to engender or facilitate the production of an effective immune response in at least some subjects. Without being bound by any particular theory or mode of action, it is proposed that the present chimeric proteins stabilise the three dimensional structure of the polypeptide or peptide and provide improved proteins and/or VLPs for effective immune response production, for antibody and in particular neutralising antibody production and for immune response and antibody screening. In some embodiments, at least an antigenic portion of a non-FV viral envelope protein is employed as a POI. In some embodiments, a viral peptide derived from a viral envelope or a non-viral envelope protein comprising an epitope that stimulates the production of neutralising antibodies may be employed. In other embodiments, the invention permits identification of new conformational epitopes recognised by neutralising antibodies from infected subjects.

An “antigen” or “immunogen” or “antigenic” or “immunogenic” refers to a molecule containing one or more epitopes (either linear, conformational or both) that will stimulate an immune system to make a humoral and/or cellular antigen-specific response. Generally, a B-cell epitope will include at least about 5 amino acids but can be as small as 3-4 amino acids. A T-cell epitope, such as a cytolytic T-cell (CTL) epitope, will include at least about 7-9 amino acids, and a helper T-cell epitope at least about 12-20 amino acids. Normally, an epitope will include between about 7 and 15 amino acids, such as, 9, 10, 12 or 15 amino acids. The term “antigen” denotes both subunit antigens, (i.e., antigens which are separate and discrete from a whole organism with which the antigen is associated in nature), as well as, killed, attenuated or inactivated bacteria, viruses, fungi, parasites or other microbes. Antibodies such as anti-idiotype antibodies, or fragments thereof, and synthetic peptide mimotopes, which can mimic an antigen or antigenic determinant, are also captured under the definition of antigen as used herein. Similarly, an oligonucleotide or polynucleotide which expresses an antigen or antigenic determinant in vivo, such as in gene therapy and DNA immunization applications, is also included in the definition of antigen herein.

An “immunological response” or “immune response” to an antigen or composition is the development in a subject of a humoral and/or a cellular immune response to an antigen present in the composition of interest. The ability of a particular antigen to stimulate a cell-mediated immunological response may be determined by a number of assays, such as by lymphoproliferation (lymphocyte activation) assays, CTL cytotoxic cell assays, or by assaying for T-lymphocytes specific for the antigen in a sensitised subject. Such assays are well known in the art. Methods of measuring cell-mediated immune response include measurement of intracellular cytokines or cytokine secretion by T-cell populations, or by measurement of epitope specific T-cells. The immune response may serve to neutralize infectivity, and/or mediate antibody-complement, or antibody dependent cell cytotoxicity (ADCC) to provide protection to an immunised host. Such responses can be determined using standard immunoassays and neutralization assays, as known in the art. An “immunogenic composition” is a composition that comprises an antigenic molecule where administration of the composition to a subject results in the development in the subject of a humoral and/or a cellular immune response to the antigenic molecule of interest. In some embodiments, the immune response includes the production of neutralising antibodies capable of reducing pathogen infectivity, pathogen transmission, and/or pathogen load in a subject.

In some illustrative embodiments, the antigen, POI or non-foamy virus virus envelope protein comprises or is derived from HIV-1 gp120, HIV-2 gp125, HIV-1 gp41, HIV-2 gp36 or a functional variant and/or antigenic portion thereof.

In some illustrative embodiments, the POI or non-foamy virus virus envelope protein is HA of influenza virus or a functional variant and/or antigenic portion thereof.

In some embodiments, the POI or non-foamy virus virus envelope protein is SARS S1 protein or a functional variant or antigenic portion thereof. SARS coronavirus only naturally forms VLPs together with structural proteins M and E. It does not form VLPs with the spike (S) protein alone.

In some embodiments, the POI or non-foamy virus virus envelope protein is RSV F2 protein or a functional variant and/or antigenic portion thereof.

In some embodiments, the POI or non-foamy virus virus envelope protein is Dengue Virus E protein or a functional variant and/or antigenic portion thereof.

The POI or viral envelope polypeptide or peptide may be positioned at either one or both side/s (N-terminal or C-terminal) of the trimer forming portion of FV TM.

As described in herein, soluble forms of the trimeric chimeric FV TM are produced wherein the membrane spanning domain and the cytoplasmic tail of FV TM are absent. A transmembrane domain is useful for efficient VLP formation and the transmembrane domain may be derived from FV TM or another source or (non-FV TM) viral envelope protein.

The cytoplasmic domain comprises an endoplasmic reticulum retrieval signal (ERRS) and removal or mutation of the ERRS diverts virus budding from the endoplasmic reticulum to the plasma membrane. Accordingly, the cytoplasmic tail may be deleted or mutated to facilitate viral budding. Alternatively, equine foamy virus may be employed which does not have an ERRS and which buds exclusively from the plasma membrane.

In some embodiments, the trimer and/or VLP forming portion of transmembrane protein of foamy virus envelope protein comprises:
i) full length foamy virus transmembrane protein;
ii) foamy virus transmembrane protein absent a functional cytoplasmic domain;
iii) foamy virus transmembrane protein absent a functional cytoplasmic domain and transmembrane domain;
iv) foamy virus ectodomain comprising N-terminal heptad repeat region and cysteine rich region between N-terminal heptad repeat region and C-terminal α-helical region;
v) N-terminal heptad repeat region;
vi) a functional variant of any one of i) to v) optionally having conservative substitutions therein; or
vii) any one of i) to vi) lacking an FV fusion peptide domain.

The “cysteine rich region” between the N-terminal heptad repeat region and the C-terminal α-helical region comprises seven cysteines at amino acid positions 181, 192, 200, 209, 218, 249 and 267. In some embodiments, at least 2, 3, 4, 6 or 7 of these cysteines are retained in the region together with interspersed amino acids or conservative substitutions thereof. It is proposed that the two or more of the cysteines facilitate VLP formation or the stability of trimers/oligomers.

In some embodiments, the present invention provides chimeric trimeric proteins comprising at least a trimer forming portion of FV TM and a POI.

In one embodiment, the present invention provides a chimeric protein comprising a protein of interest (POI) bound to a transmembrane protein (TM) of a FV envelope protein or a functional variant of a transmembrane protein (TM) of a FV envelope protein (collectively herein referred to as “FV TM”).

In particular embodiments, the chimeric proteins form dimers, trimers or oligomers at least in part through interactions of the protein of interest (POI) with FV TM. In particular embodiments, the invention provides chimeric viral envelope proteins wherein the protein of interest is a viral envelope protein or a functional variant thereof.

Reference herein to “bound” includes covalent and non-covalent bonds. In illustrated embodiments, the bond is a covalent bond, such as between components of a fusion protein. “Fused” refers to a covalent bond. In some embodiments, chimeric proteins are in the form of a trimer or a complex comprising a trimeric antigen or higher order multimers or olimeric forms of the trimeric protein.

In some embodiments, the present invention provides a chimeric protein comprising a surface protein (SU) of a non-foamy virus (non-FV) virus envelope protein bound or fused to a FV TM. Accordingly, in some embodiments the POI is a surface protein (SU) of a non-foamy virus (non-FV) virus envelope protein. In some embodiments, the non-FV viral envelope protein is recognised by a neutralising or conformational antibody.

Reference herein to “functional variants” of FV TM include naturally or non-naturally occurring functional variants, biologically active parts or portions, precursors, derivatives, analogs and recombinant or synthetic forms having a degree of sequence similarity or the omission of one or more biologically active parts or portions sufficient to retain the functional and structural ability of the sequences identified herein to form oligomeric structures and VLPs as described herein. Functional variants are described further in the detailed description. The trimer or oligomer or forming activity of functional variants can be tested using methods routine in the art and described herein.

For example, the full length FV TM comprises various FV domains that do not appear to be essential for the purposes of the present invention, such as the FV fusion domain and the FV cytoplasmic domain and FV TM without one or more of these domains are illustrative functional variants.

In some embodiments, a FV TM comprises the amino acid sequence of the FV TM protein set out in SEQ ID NO: 3 (HFV gp47) or SEQ ID NO: 6 (HFV gp47ΔCD) or SEQ ID NO: (HA1-gp47 with ERRS K>S substitution at amino acids 413-415) or SEQ ID NO: 14 (HA1-gp47ΔCD with furin cleavage site mutated) or SEQ ID NO: 22 (HIV-1R2 gp120-FP-gp47ΔCD comprising fusion peptide of HIV) or a sequence having at least about 90% sequence similarity to one of the FV TM sequences set out in SEQ ID NO: 3 or SEQ ID NO: 6 or SEQ ID NO: 10 or SEQ ID NO: 12 or SEQ ID NO: 14, SEQ ID NO: 22, SEQ ID NO: SEQ ID NO: 24, SEQ ID NO: 26, or SEQ ID NO: 28. In some embodiments, the FV TM comprises an amino acid sequence having at least 90% similarity to the sequences set out in SEQ ID NO: 24 (heptad repeat domain) or to a functional variant thereof having one or more conservative amino acid substitutions.

In a particular embodiment, the FV gp47 protein has the amino acid sequence set out in SEQ ID NO: 3 or comprises the amino acid sequence set out in SEQ ID NO: 24.

In further particular embodiments, soluble trimeric FV TM comprises an N-terminal heptad repeat region, a C-terminal α-helical domain and a cysteine-rich intervening region. Membrane binding FV TM further comprises a transmembrane domain derived from FV TM or from a non-FV TM source. Amino acid sequences derived from HIV gp41, Influenza HA2, RSV F2, or SARS S2 transmembrane domains may, for example, be employed. In some embodiments, the FV TM comprises FV TM domains including one or more of a fusion peptide domain, a membrane (transmembrane) spanning domain, a C-terminal α-helical region, a cytoplasmic tail region, and a polypeptide or peptide of interest. An illustrative peptide for inclusion within FV TM is the membrane proximal region of HIV. The skilled artisan is now able to routinely modify the subject constructs to produce constructs with one or more of these domains and functional variants thereof.

In some embodiments, the chimeric protein is recognised by anti-POI neutralising antibody. In an illustrative example, the trimeric HIV gp120-FV gp47 proteins are recognised by conformational anti-HIV MAb b12 and/or MAb 2G12. In another illustrative example, the trimeric HA-gp47 proteins are recognised by a conformational anti-HA antibody, MAb 149.

In some embodiments, the transmembrane protein is produced lacking a transmembrane domain and cytoplasmic tail. However, membrane attached forms are also expressly contemplated and exemplified herein in the form of VLPs. Unlike other retroviruses, envelope proteins of FV assemble into subviral particles without requiring other viral structural such as gag polypeptide.

“Similarity” refers to the percentage number of amino acids that are identical or constitute conservative substitutions as defined in Table 4. Similarity may be determined using sequence comparison programs such as GAP (Deveraux et al., Nucleic Acids Research 12: 387-395, 1984). In this way, sequences of a similar or substantially different length to those cited herein might be compared by insertion of gaps into the alignment, such gaps being determined, for example, by the comparison algorithm used by GAP. Gap comparisons may not be appropriate where the position of a particular residue is critical in determining function and in this context un-gapped comparisons are performed, as known in the art.

The term “sequence identity” as used herein refers to the extent that sequences are identical on a nucleotide-by-nucleotide basis or an amino acid-by-amino acid basis over a window of comparison. Thus, a “percentage of sequence identity” is calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. For the purposes of the present invention, “sequence identity” will be understood to mean the “match percentage” calculated by an appropriate method. For example, sequence identity analysis may be carried out using the DNASIS computer program (Version 2.5 for windows; available from Hitachi Software engineering Co., Ltd., South San Francisco, Calif., USA) using standard defaults as used in the reference manual accompanying the software.

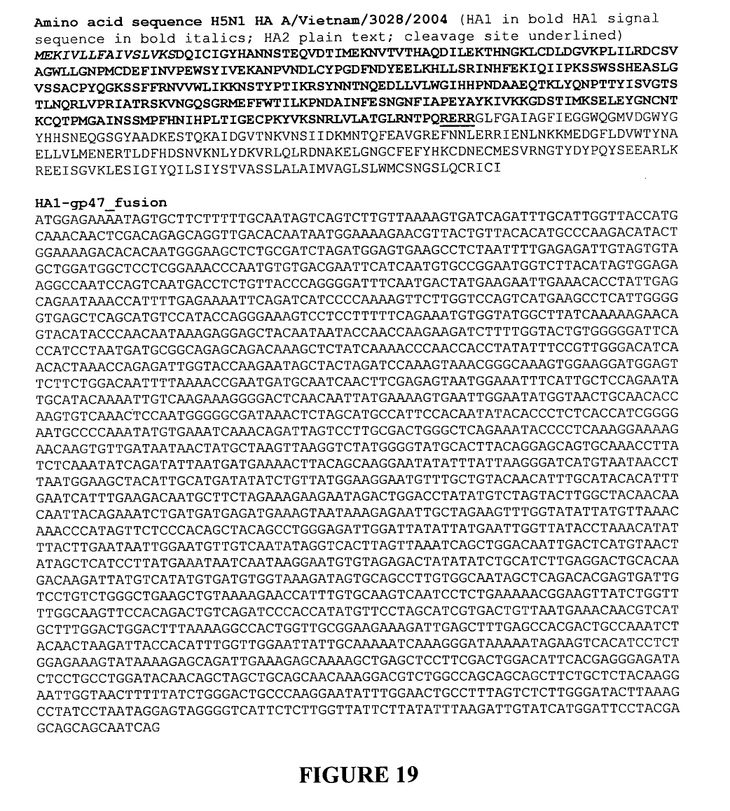

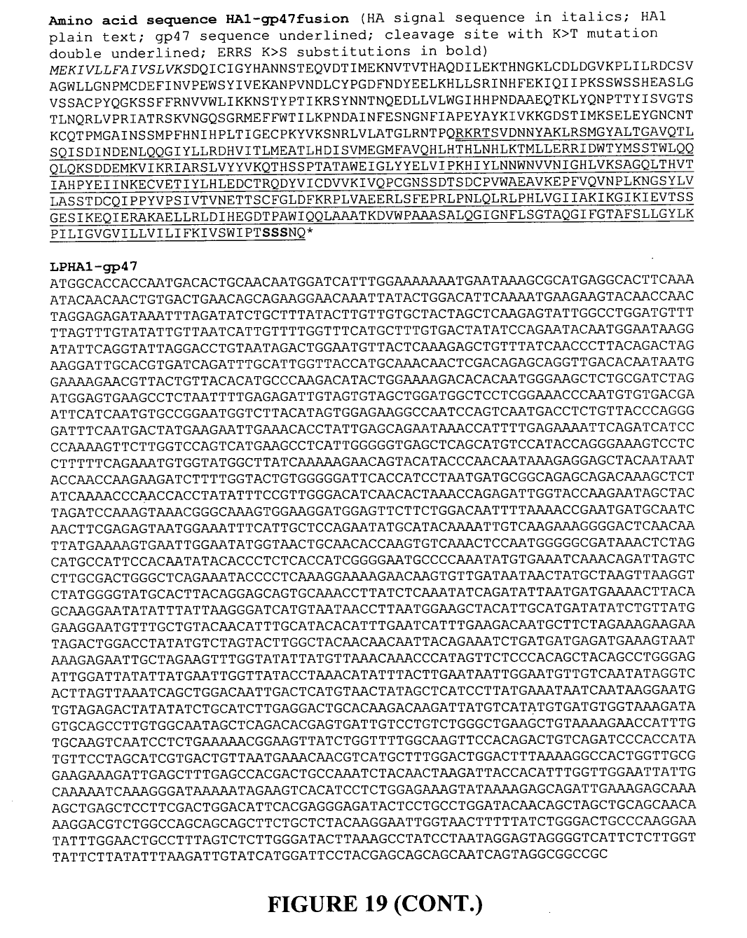

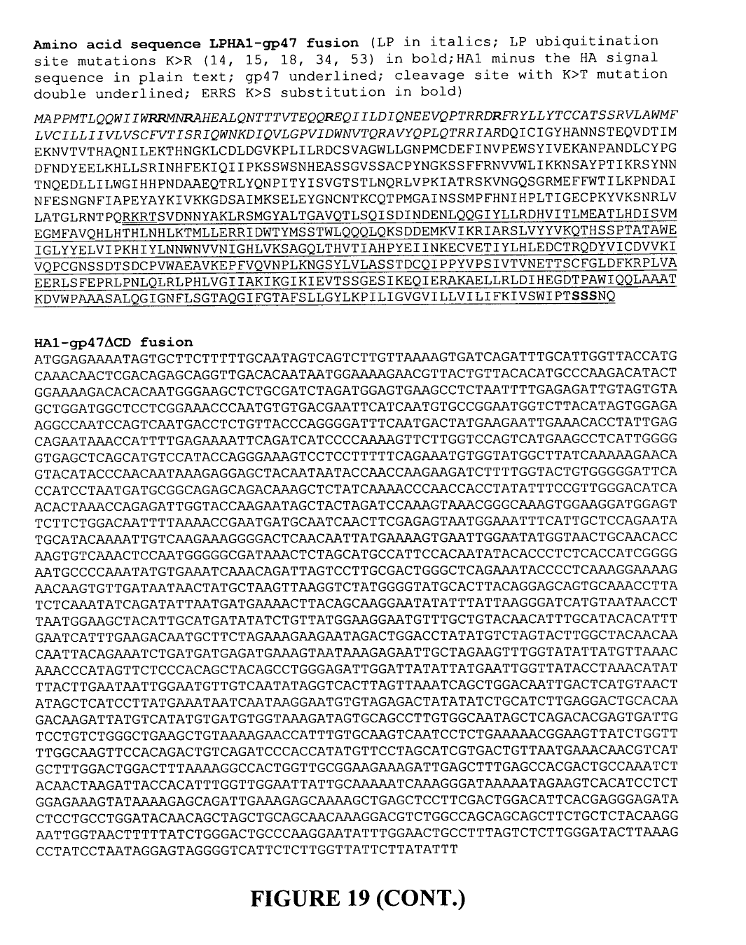

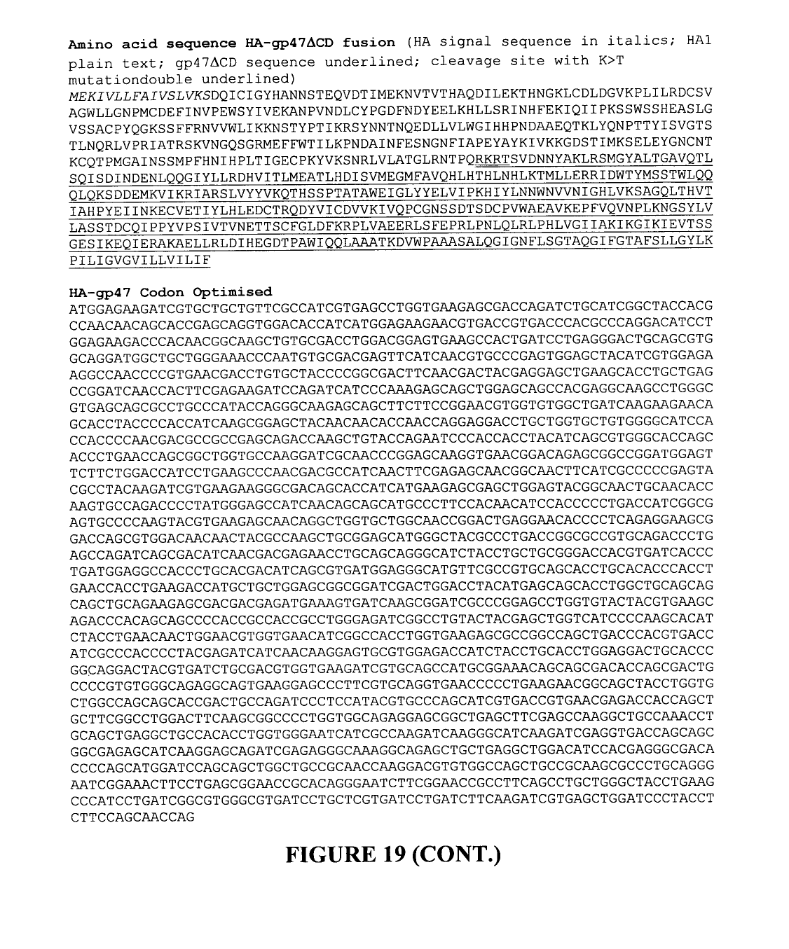

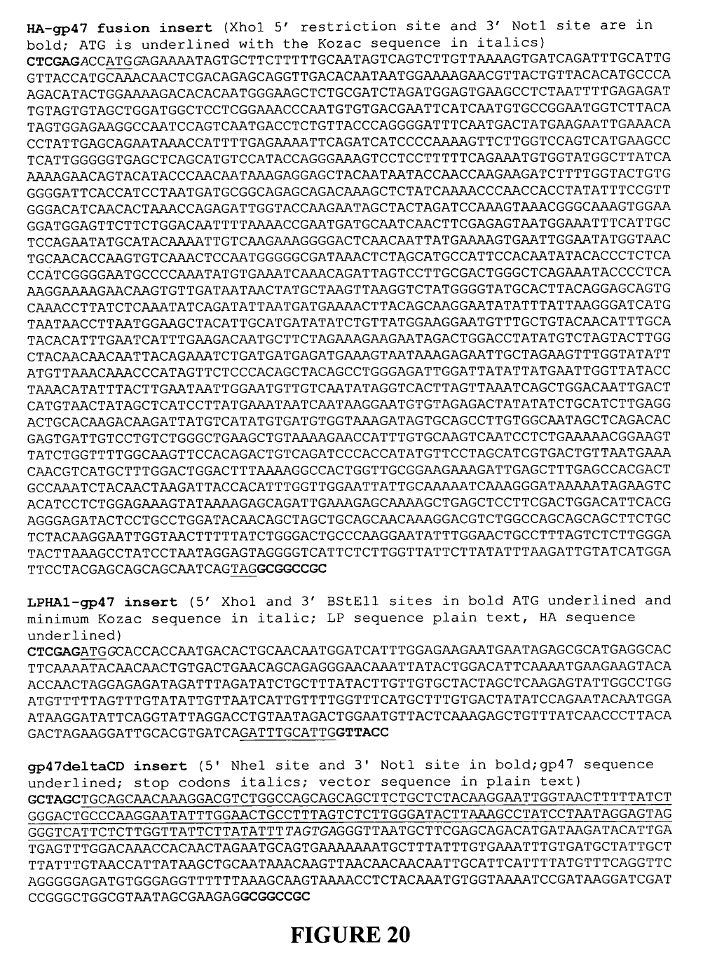

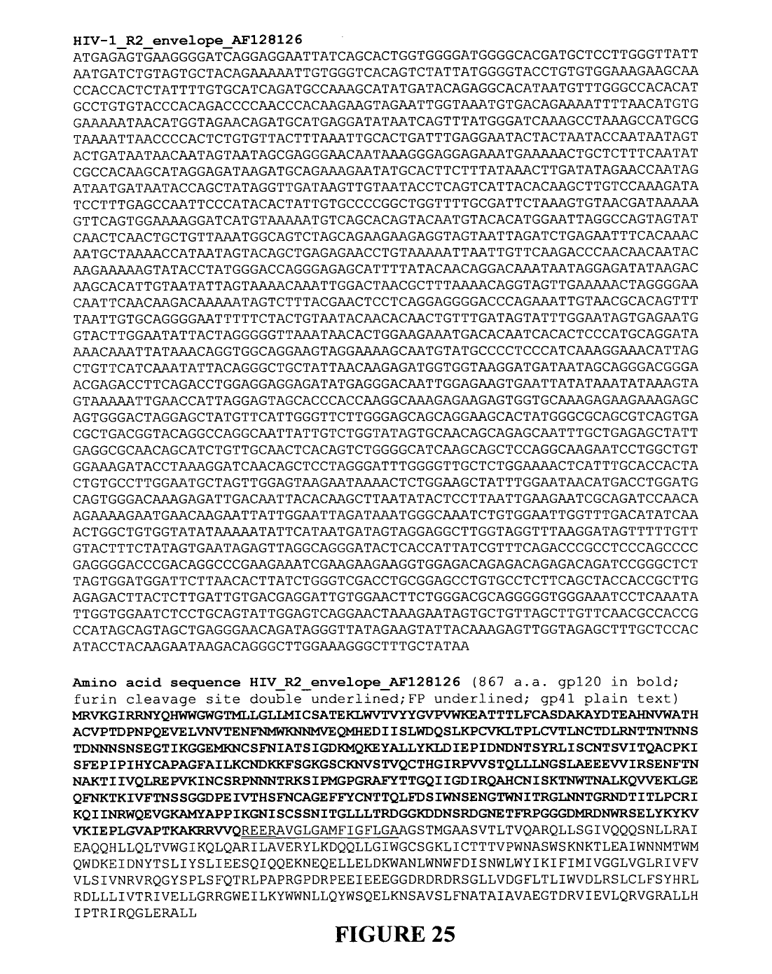

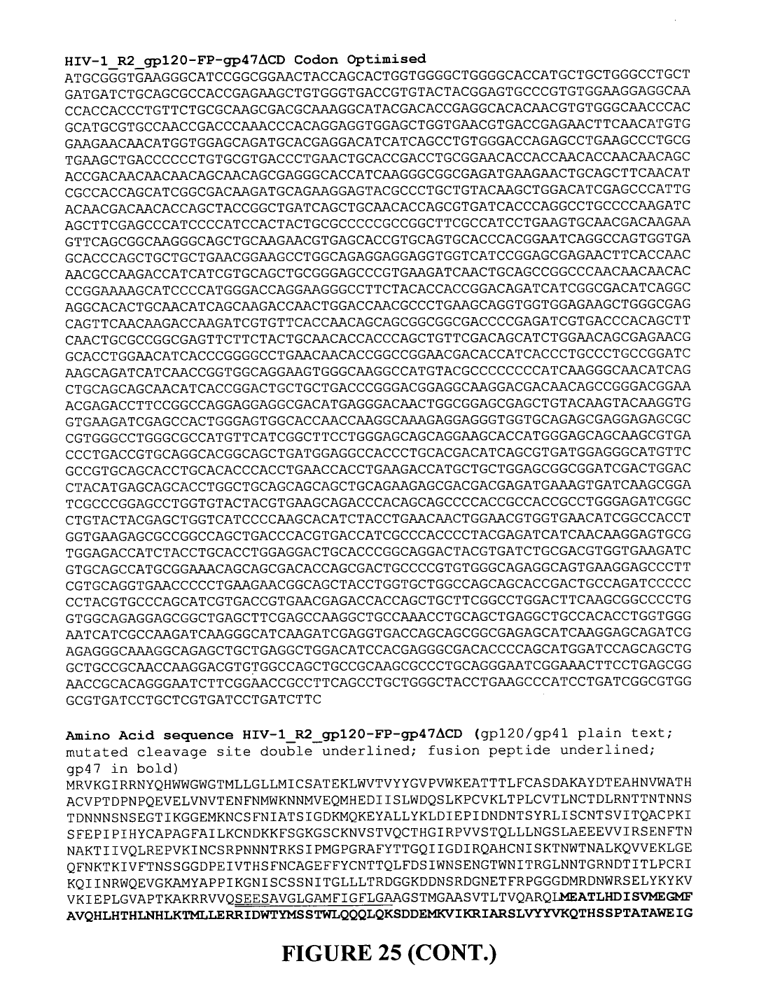

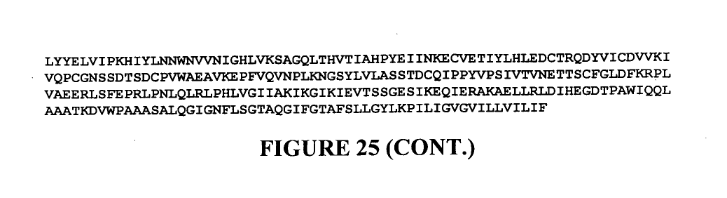

In some embodiments, the FV TM comprises an amino acid sequence of FV TM encoded by a sequence of nucleotides set forth in SEQ ID NO: 2, SEQ ID NO: 9, SEQ ID NO: 11, SEQ ID NO: 13, SEQ ID NO: 15, SEQ ID NO: 18, SEQ ID NO: 21 or SEQ ID NO: 23, SEQ ID NO: 25 or SEQ ID NO: 27 further described and illustrated in FIG. 3, FIGS. 12, 19, 20 and 25, or corrected versions thereof or a sequence of nucleotides having at least 90% sequence identity thereto or a sequence of nucleotides that hybridises to a complementary form of FV TM encoding sequences under conditions of medium stringency.

In some embodiments, the FV TM comprises FV gp47 absent FV fusion peptide, absent membrane spanning domain (MSP), and absent the cytoplasmic tail (CD) (illustrated in the amino acid sequence set forth in SEQ ID NO: 35) and is encoded by the sequence of FV TM nucleotides set out in SEQ ID NO: 25. In other embodiments, the C-terminal α-helical region is further absent (see, for example, the amino acid sequence of an N-terminal heptad repeat domain and a cysteine rich region as set forth in SEQ ID NO: 36).

Terms used to describe sequence relationships between two or more polynucleotides or polypeptides include “reference sequence”, “comparison window”, “sequence identity”, “percentage of sequence identity” and “substantial identity”. A “reference sequence” is at least 12 but frequently 15 to 18 and often at least 25 monomer units, inclusive of nucleotides and amino acid residues, in length. Because two polynucleotides may each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of at least 6 contiguous positions, usually about 50 to about 100, more usually about 100 to about 150 in which a sequence is compared to a reference sequence of the same number of contiguous positions after the two sequences are optimally aligned. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerised implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also may be made to the BLAST family of programs as for example disclosed by Altschul et al., Nucl. Acids Res., 25: 3389-3402, 1997. A detailed discussion of sequence analysis can be found in Unit 19.3 of Ausubel et al., Current Protocols in Molecular Biology, John Wiley & Sons Inc, Chapter 15, 1994-1998.

“Synthetic” sequences, as used herein, include polynucleotides whose expression has been optimised as described herein, for example, by codon substitution, deletions, replacements and/or inactivation of inhibitory sequences. “Wild-type” or “native” or “naturally occurring” sequences, as used herein, refers to polypeptide encoding sequences that are essentially as they are found in nature. The subject polynucleotide sequences may be codon optimised as illustrated herein.

In some embodiments, the chimeric protein lacks a functional cleavage site for host cellular proteinases between the POI and FV TM. In an illustrative embodiment, the N-terminal furin cleavage site in FV TM is changed from RKRR to RKRT. In other embodiments, the fusion peptide domain of a non-FV envelope protein is employed and the furin cleavage site disabled. In an illustrative embodiment, the non-FV virus envelope protein is HIV gp120 and the fusion peptide domain of HIV gp41 is employed. In this embodiment, the furin cleavage site REER is changed to SEES. In this embodiment, the fusion peptide domain is bound or fused to the N-terminal heptad repeat of FV TM minus the cytoplasmic domain, as described in the Examples.

In some embodiments, the FV TM lacks a transmembrane domain and cytoplasmic tail. Accordingly, in some embodiments, the protein is soluble and readily secreted into media.

The invention further provides trimeric FV TM without a POI, as described in the Examples, and methods for producing same. See, for example, HAtag-gp47 which comprises an HA tag for detection purposes but which also forms a trimer.

Further, the invention provides trimeric chimeric FV TM comprising a POI (see FIG. 47) and methods for producing same.

The present invention provides inter alia methods for producing a soluble trimeric viral envelope protein such as trimeric HIV gp120, HIV-2 gp125, SARS S1, Dengue virus E protein, RSV F2, Influenza HA protein, Measles M protein. The methods comprise producing a nucleic acid encoding and capable of directing the expression of a soluble (non-membrane bound) chimeric protein as defined herein, transfecting (transforming) cells with the nucleic acid and isolating trimeric protein from the cell media. The proteins are produced by culturing the host cells for a period of time sufficient to allow for expression of the protein in the host cells or, more preferably, secretion of the protein into the culture medium in which the host cells are grown.

In other embodiments, the invention provides a host cell or cell membrane preparation, chimeric VLP or proteoliposome, each comprising or encoding a chimeric protein of the present invention including a chimeric protein comprising a surface protein (SU) of a non-foamy virus (non-FV) virus envelope protein or a functional variant thereof bound or fused to a transmembrane protein (TM) of a FV envelope protein or a functional variant thereof. Methods for making proteoliposomes are described in the art.

In some embodiments, the cell is a host cell for expression of chimeric protein such as eukaryotic cell, preferably a yeast, avian, insect, plant or mammalian cells. In other embodiments, the cell is the cell of a subject to be treated.

A “coding sequence” or a sequence which “encodes” a selected polypeptide, is a nucleic acid molecule which is transcribed (in the case of DNA) and translated (in the case of mRNA) into a polypeptide in vivo when placed under the control of appropriate regulatory sequences (or “control elements”). The boundaries of the coding sequence are determined by a start codon at the 5′ (amino) terminus and a translation stop codon at the 3′ (carboxy) terminus. A coding sequence can include, but is not limited to, cDNA from viral, prokaryotic or eukaryotic mRNA, genomic DNA sequences from viral or prokaryotic DNA, and synthetic DNA sequences. A transcription termination sequence may be located 3′ to the coding sequence.

Typical “control elements”, which may be employed to provide expression include, but are not limited to, transcription promoters, transcription enhancer elements, transcription termination signals, polyadenylation sequences (located 3′ to the translation stop codon), sequences for optimization of initiation of translation (located 5′ to the coding sequence), and translation termination sequences.

“Operably linked” refers to an arrangement of elements wherein the components so described are configured so as to perform their usual function. Thus, a given promoter operably linked to a coding sequence is capable of effecting the expression of the coding sequence when the proper enzymes are present. The promoter need not be contiguous with the coding sequence, so long as it functions to direct the expression thereof.

The term “recombinant” may be used herein to describe a nucleic acid molecule and means a polynucleotide of genomic, cDNA, semisynthetic, or synthetic origin which, by virtue of its origin or manipulation: (1) is not associated with all or a portion of the polynucleotide with which it is associated in nature; and/or (2) is linked to a polynucleotide other than that to which it is linked in nature. The term “recombinant” as used with respect to a protein or polypeptide means a polypeptide produced by expression of a recombinant polynucleotide.

“Recombinant host cells,” “host cells,” “cells,” “cell lines,” “cell cultures,” and other such terms denoting prokaryotic microorganisms or eukaryotic cell lines cultured as unicellular entities, are used interchangeably, and refer to cells which can be, or have been, used as recipients for recombinant vectors or other transfer DNA, and include the progeny of the original cell which has been transfected.

The production and characteristics of soluble HA1-gp47 trimers, soluble HIV-1 gp120-FVgp47 trimers and soluble HAtag-gp47 trimers are described in detail in the Examples.

The production and characterisation of membrane bound trimeric chimeric proteins or trimeric FV TM alone proteins are also within the scope of the present invention. These proteins comprise a membrane spanning domain.

In another embodiment, the present invention also provides a method for producing a protein or a VLP, the method comprising producing a nucleic acid construct comprising a sequence encoding protein of interest (POI) connected in frame to a sequence encoding a at least a trimer forming or a trimer and VLP forming portion of transmembrane protein (gp47) of foamy virus (FV) or a functional variant thereof and introducing same into an expression vector and expressing same in a suitable cell. The sequences encoding the POI may be N-terminal or C-terminal to the trimer forming portion of FV TM.

In another embodiment, the present invention also provides a method for producing a protein or a VLP comprising same, the method comprising producing a nucleic acid construct comprising a 5′ sequence encoding protein of interest (POI) connected in frame to a 3′ sequence encoding a transmembrane protein (gp47) of foamy virus (FV) or a functional variant thereof; and introducing same into an expression vector and expressing same. In accordance with the present invention, the polynucleotide sequence encoding the FV TM comprises sequence encoding a trimer forming portion of FV TM.

In some embodiments, the present invention provides nucleic acid molecules encoding the chimeric proteins of the present invention. In some embodiments, the present invention provides a nucleic acid molecule comprising one of the polynucleotide sequences set out in SEQ ID NO: 9, SEQ ID NO: 11, SEQ ID NO: 13, SEQ ID NO: 15, SEQ ID NO: 16, SEQ ID NO: 17, SEQ ID NO: 18, SEQ ID NO: 21, SEQ ID NO: 23, and SEQ ID NO: 25 or corrected or codon optimised versions thereof or a sequence of nucleotides having at least 90% sequence identity thereto or a sequence of nucleotides that hybridises to a complementary form under conditions of medium stringency. The polynucleotide sequences encode a chimeric polypeptide comprising a POI and FV TM in accordance with the present invention.

In another embodiment, the invention provides a nucleic acid molecule comprising one of the polynucleotide sequences set out in SEQ ID NO: 27 or a corrected or codon optimised version thereof or a sequence of nucleotides having at least 90% sequence identity thereto or a sequence of nucleotides that hybridises to a complementary form under conditions of medium stringency. The polynucleotide sequence encodes HAsigHAtag-gp47 described in Examples 17 to 19.

In some embodiments, the FV TM comprises FV gp47 absent FV fusion peptide, absent membrane spanning domain (MSP), and absent the cytoplasmic tail (CD) (amino acid sequence SEQ ID NO: 35) and is encoded by the sequence of FV TM nucleotides set out in SEQ ID NO: 25. In other embodiments, the C-terminal α-helical region is also absent (providing the amino acid set out in SEQ ID NO: 36).

In some embodiments, polynucleotide sequences encode a chimeric polypeptide comprising a FV TM and a POI wherein the FV TM comprises:
i) full length foamy virus transmembrane protein;
ii) foamy virus transmembrane protein absent a functional cytoplasmic domain;
iii) foamy virus transmembrane protein absent a transmembrane domain or absent a functional cytoplasmic domain and a transmembrane domain;
iv) foamy virus ectodomain comprising N-terminal heptad repeat region and cysteine rich region between N-terminal heptad repeat region and C-terminal α-helical region;
v) N-terminal heptad repeat region;
vi) a functional variant of any one of i) to v) optionally having conservative substitutions therein; or
vii) any one of i) to vi) lacking an FV fusion peptide domain.

In other embodiments, the present invention provides nucleic acid molecules encoding and capable of producing the presently described chimeric proteins in a range of cells, in vitro or in vivo.

In another embodiment, the present invention provides an isolated nucleic acid molecule encoding a chimeric protein wherein the chimeric protein comprises a surface protein (SU) of a non-foamy virus (non-FV) virus envelope protein or a functional variant thereof bound or fused to a transmembrane protein (TM) of a FV envelope protein or a functional variant thereof.

In some embodiments, the nucleic acid molecules are introduced into expression systems to facilitate expression of chimeric proteins in vitro or in vivo.

Any suitable expression system can be employed including stable cell lines and transient expression vectors, CMV-promoter-based mammalian vectors, and a shuttle vector for use in baculovirus expression systems.

Suitable mammalian cell lines include, but are not limited to, BHK, VERO, HT1080, 293, 293T, RD, COS-7, CHO, Jurkat, HUT, SUPT, C8166, MOLT4/clone8, MT-2, MT-4, H9, PM1, CEM, myeloma cells (e.g., SB20 cells) and CEMX174 are available, for example, from the ATCC).

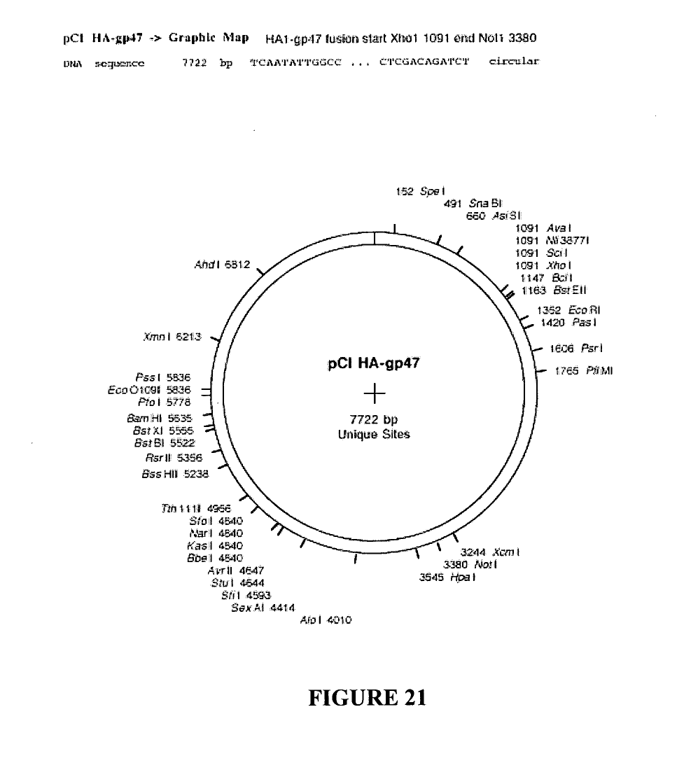

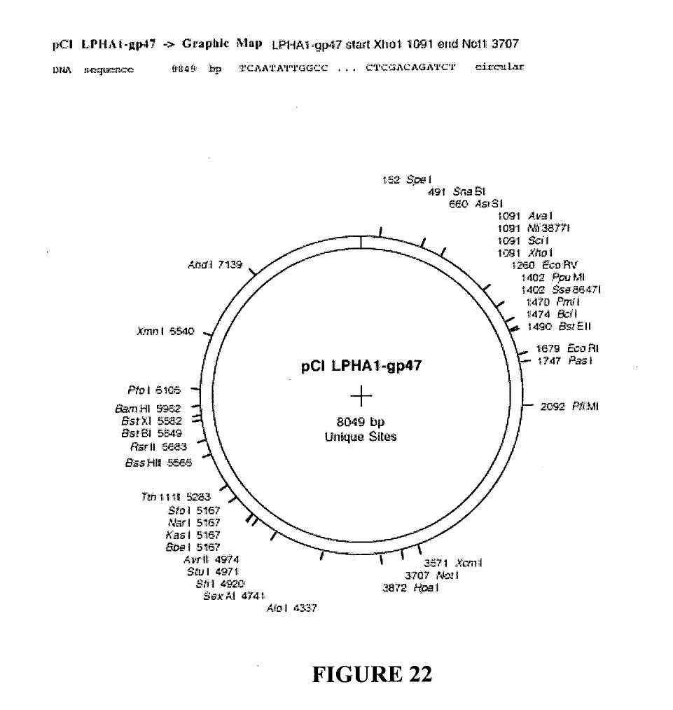

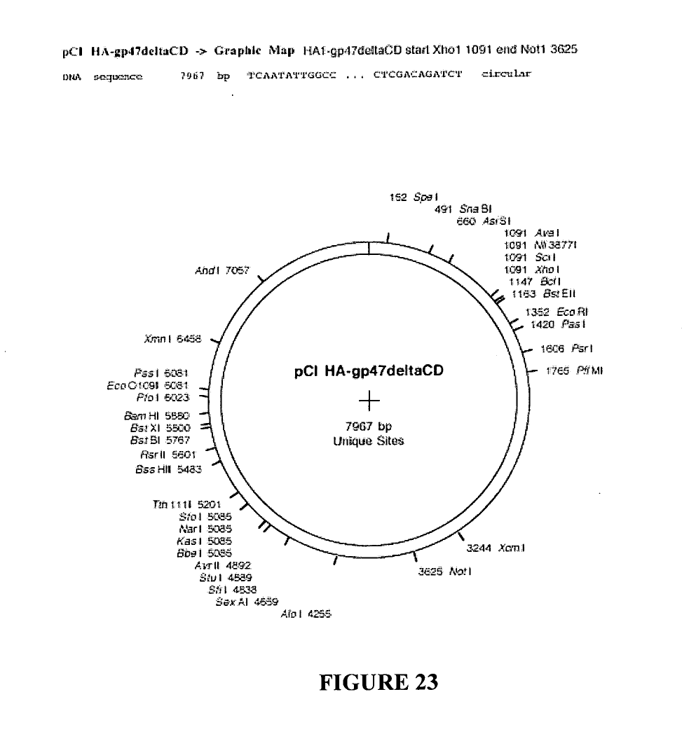

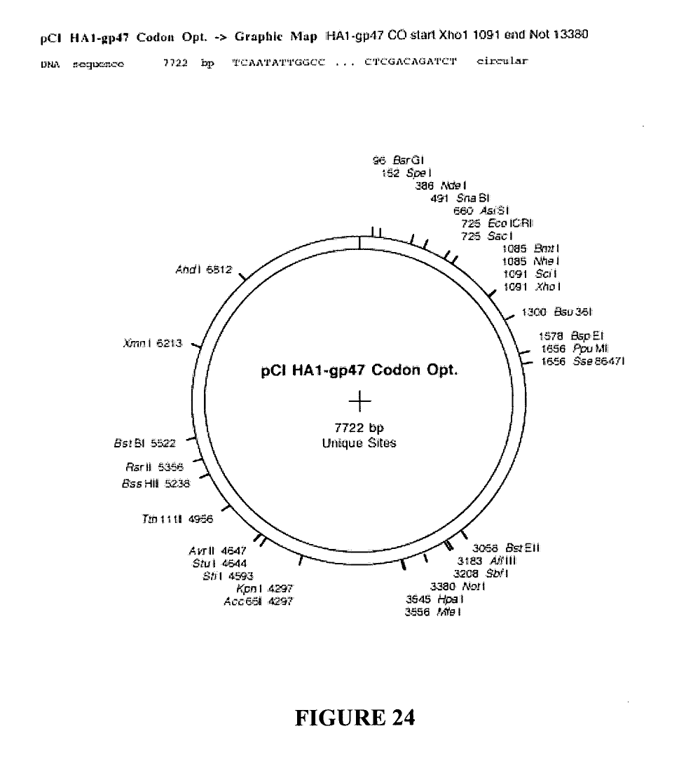

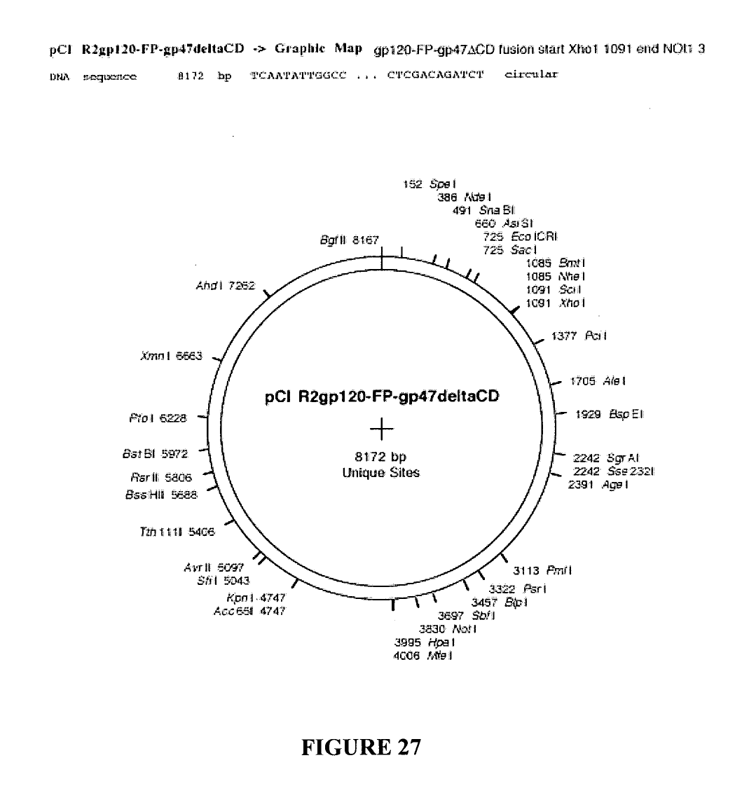

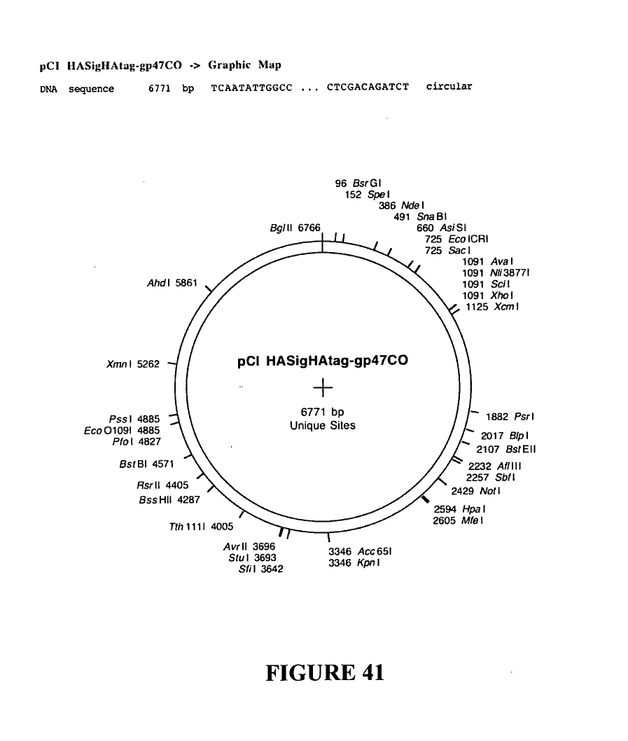

The synthetic DNA may be recombinantly expressed by molecular cloning into an expression vector containing a suitable promoter and other appropriate transcription regulatory elements, and transferred into prokaryotic or eukaryotic host cells to produce recombinant protein. Techniques for such manipulations are fully described by Sambrook et al., 1989 (supra); Ausubel et al., Current Protocols in Molecular Biology, Green Pub. Associates and Wiley-Interscience, New York, 1988. Plasmid maps for illustrative plasmids are provided in FIGS. 21, 22, 23, 24, 27, 35, and 41.

In some non-limiting embodiments, the present invention provides a method for expressing a chimeric protein in a recombinant host cell, comprising: (a) introducing a vector comprising a nucleic acid encoding an a chimeric protein into a yeast host cell; and (b) culturing the yeast host cell under conditions which allow expression of said protein. For example, construct for expression in yeast preferably contains the synthetic gene, with related transcriptional and translations control sequences operatively linked to it, such as a promoter (such as GAL10, GALT, ADH1, TDH3 or PGK), and termination sequences (such as the S. cerevisiae ADH1 terminator). The yeast may be selected from the group consisting of: Saccharomyces cerevisiae, Haisenula polymorpha, Pichia pastoris, Kluyveromyces fragilis, Kluyveromyces lactis, and Schizosaccharomyces pombe. See also Yeast Genetics: Rose et al., A Laboratory Course Manual, Cold Spring Harbor Laboratory, Cold Spring Harbor, N.Y., 1990. Nucleic acid molecules can be codon optimised for expression in yeast as known in the art (see Sharp and Cowe, Yeast, 7: 657-678, 1991).

Appropriate vectors and control elements for any given cell type can be selected by one having ordinary skill in the art in view of the teachings of the present specification and information known in the art about expression vectors.

Translational control elements have been reviewed by M. Kozak (e.g., Kozak, Mamm Genome, 7(8): 563-74, 1996; Kozak, Biochimie., 76(9):815-21, 1994; Kozak, J Cell Biol, 108(2): 229-241, 1989; Kozak and Shatkin, Methods Enzymol, 60: 360-375, 1979).

Purification can be carried out by methods known in the art including salt fractionation, ion exchange chromatography, gel filtration, size-exclusion chromatography, size-fractionation, and affinity chromatography. Immunoaffinity chromatography can be employed using antibodies generated based on, for example, envelope antigens.

Chimeric polypeptides may be prepared by any suitable procedure known to those of skill in the art. For example, chimeric fusion polypeptides may be prepared by a procedure including the steps of: (a) preparing a construct comprising a polynucleotide sequence that encodes the polypeptide and that is operably linked to a regulatory element; (b) introducing the construct into a host cell; (c) culturing the host cell to express the polypeptide; and (d) isolating the polypeptide from the host cell. In illustrative examples, the nucleotide sequence encodes at least a biologically active portion of the sequence set forth in any one of SEQ ID NO: 3, 6, 10, 12, 14, 22, 14, 26, 28 or a variant thereof. Recombinant polypeptides can be conveniently prepared using standard protocols as described for example in Sambrook, et al., 1989 (supra), in particular Sections 16 and 17; Ausubel et al., 1994 (supra), in particular Chapters 10 and 16; and Coligan et al., Current Protocols in Protein Science, John Wiley & Sons, Inc. 1995-1997, in particular Chapters 1, 5 and 6. The polypeptides or polynucleotides may be synthesised by chemical synthesis, e.g., using solution synthesis or solid phase synthesis as described, for example, in Chapter 9 of Atherton and Shephard (supra) and in Roberge et al., Science, 269(5221): 202-204, 1995.

As mentioned above, envelope proteins of FV assemble into sub-viral particles without requiring other viral structural proteins such as gag polypeptide. In accordance with the present invention it has been further identified that FV TM alone is able to form trimers and VLPs (see Examples 17 to 19).

In another aspect of the present invention, viral-like particles (VLP) are provided comprising a chimeric protein comprising a surface protein (SU) of a non-foamy virus (non-FV) virus envelope protein bound or fused to a transmembrane protein (TM) of a FV envelope protein. In particular embodiments, the FV transmembrane protein comprises a transmembrane domain for VLP formation. As shown in the Examples and Figures, HA1-FVgp47 and gp120/gp41-FVgp47 trimers of the expected molecular mass are produced and VLPs comprising said trimers are also produced.

Various variants of FV-TM are contemplated to modulate inter alia protein production and secretion.

In some embodiments, for example, the chimeric protein lacks a functional cytoplasmic domain. In some embodiments, the chimeric protein lacks an endoplasmic reticulum (ER) retrieval signal. Accordingly, as show in Example 2, the LPHA1-gp47 construct comprising an N-terminal leader peptide (LP) showed instability and/or misfolding. In some embodiments, the chimeric protein lacks a FV leader peptide.

Variants of HA or gp120 are contemplated inter alia to modulate an immune or antibody response to the chimeric polypeptide or to enhance the trimer, oligomer or VLP formation.

In some embodiments, the present invention provides a viral-like particle comprising a trimeric chimeric protein as described herein.

In a related embodiment, it is proposed to use trimeric chimeric proteins comprising FV TM and a POI and, in addition to employ FV TM trimers that do not comprise a POI that is a viral envelope protein. In some embodiments, such mixed trimer VLPs are useful for spacing out trimeric POI in the VLP to increase VLP immunogenicity and/or stability or half-life. The optimum ratio of POI-trimers and non-POI trimers can be established by routine methods. In an illustrative embodiment, the POI is HIV gp120 or influenza HA1. The production and testing of VLPs is standard in the art and methods such as those described in the specification are employed.

“Hybridization” or “hybridise” is used herein to denote the pairing of complementary nucleotide sequences to produce a DNA-DNA hybrid or a DNA-RNA hybrid. Hybridization can occur under varying circumstances as known to those of skill in the art. The phrase “hybridizing specifically to” and the like refer to the binding, duplexing, or hybridizing of a molecule only to a particular nucleotide sequence under stringent conditions.

“Stringent conditions” as used herein refers to temperature and ionic conditions under which only polynucleotides having a high proportion of complementary bases, preferably having exact complementarity, will hybridize. The stringency required is nucleotide sequence dependent and depends upon the various components present during hybridization, and is greatly changed when nucleotide analogues are used. Generally, stringent conditions are selected to be about 10° C. to 20° C. less than the thermal melting point (Tm) for the specific sequence at a defined ionic strength and pH. The Tm is the temperature (under defined ionic strength and pH) at which 50% of a target sequence hybridizes to a complementary probe.

It will be understood that a polynucleotide will hybridize to a target sequence under at least low stringency conditions, preferably under at least medium stringency conditions and more preferably under high stringency conditions. Reference herein to low stringency conditions include and encompass from at least about 1% v/v to at least about 15% v/v formamide and from at least about 1 M to at least about 2 M salt for hybridization at 42° C., and at least about 1 M to at least about 2 M salt for washing at 42° C. Low stringency conditions also may include 1% Bovine Serum Albumin (BSA), 1 mM EDTA, 0.5 M NaHPO4 (pH 7.2), 7% SDS for hybridization at 65° C., and (i) 2×SSC, 0.1% SDS; or (ii) 0.5% BSA, 1 mM EDTA, 40 mM NaHPO4 (pH 7.2), 5% SDS for washing at room temperature. Medium stringency conditions include and encompass from at least about 16% v/v to at least about 30% v/v formamide and from at least about 0.5 M to at least about 0.9 M salt for hybridization at 42° C., and at least about 0.5 M to at least about 0.9 M salt for washing at 42° C. Medium stringency conditions also may include 1% Bovine Serum Albumin (BSA), 1 mM EDTA, 0.5 M NaHPO4 (pH 7.2), 7% SDS for hybridization at 65° C., and (i) 2×SSC, 0.1% SDS; or (ii) 0.5% BSA, 1 mM EDTA, 40 mM NaHPO4 (pH 7.2), 5% SDS for washing at 42° C. High stringency conditions include and encompass from at least about 31% v/v to at least about 50% v/v formamide and from at least about 0.01 M to at least about 0.15 M salt for hybridization at 42° C., and at least about 0.01 M to at least about 0.15 M salt for washing at 42° C. High stringency conditions also may include 1% BSA, 1 mM EDTA, 0.5 M NaHPO4 (pH 7.2), 7% SDS for hybridization at 65° C., and (i) 0.2×SSC, 0.1% SDS; or (ii) 0.5% BSA, 1 mM EDTA, 40 mM NaHPO4 (pH 7.2), 1% SDS for washing at a temperature in excess of 65° C. Other stringent conditions are well known in the art. A skilled addressee will recognize that various factors can be manipulated to optimize the specificity of the hybridization. Optimization of the stringency of the final washes can serve to ensure a high degree of hybridization. For detailed examples, see CURRENT PROTOCOLS 1N MOLECULAR BIOLOGY (supra) at pages 2.10.1 to 2.10.16 and Sambrook, et al., 1989 (supra) at sections 1.101 to 1.104.

The present invention provides methods for inducing a specific immune response in a subject in need comprising administering a chimeric protein comprising a POI and a trimer forming portion of FV TM, a viral-like particle or a nucleic acid encoding the chimeric protein of the present invention as described herein.

“Subjects” contemplated in the present invention include any animal of commercial or humanitarian interest including conveniently, primates, livestock animals including fish, crustacea, and birds, laboratory test animals, companion animals, or captive wild animals. In some embodiments the subject is a mammalian animal. In particular embodiments, the subject is a human subject.

The present invention provides methods for producing neutralising antibodies to a pathogen in an animal comprising administering a chimeric protein, a viral-like particle or a nucleic acid of the present invention as described herein to the animal and selecting antibodies therefrom that are able to bind to the POI of the pathogen. In a particular embodiment, the POI is a viral envelope protein such as HIV gp120 or influenza HAL Antibodies are tested, in some embodiments, for their ability to reduce virus infectivity or viral load.

The invention further provides methods of screening for antibodies or other agents that specifically bind trimeric viral envelope polypeptide comprising contacting a sample or solution comprising an antibody or other agent with a chimeric trimeric or higher oligomer-protein as described herein and determining binding relative to controls. Binding agents are then tested for their therapeutic or prophylactic ability for example to reduce infectivity, viral load or transmission.

The present invention contemplates a method of screening, the method comprising contacting a putative interacting compound with a chimeric protein comprising a POI and a trimer forming portion of FV TM as described herein or a viral-like particle comprising same; and determining binding characteristics of an interaction between the putative interacting compound and the POI of the chimeric protein.

The present invention contemplates a method of screening, the method comprising contacting a putative interacting compound with a chimeric protein comprising a surface (SU) of a non-foamy virus (non-FV) virus envelope protein bound to a transmembrane (TM) of a FV envelope protein, or a viral like particle comprising same; and determining binding characteristics of an interaction between the putative interacting compound and the chimeric protein.

The present invention contemplates a method comprising contacting a sample from a subject with a chimeric protein comprising a POI and a trimer forming portion of FV TM as described herein or a virus like particle comprising same; and determining an interaction between the sample and the POI of the chimeric protein. In some embodiments, arrays of different chimeric proteins comprising different POI may be employed. In some embodiments, the sample is a sample comprising antibodies.

In some embodiments, the sample is from an infected subject. Control samples include samples from uninfected individuals. A sample may be from any part of the subject. Convenient samples include blood, serum, plasma, urine, sputum and the like.

Suitable assays are known to those of skill in the art and include ELISA, RIA and EIA-like assays and competitive assays. The subject assays are particularly useful for serosurveillance.

The present invention contemplates a method comprising contacting a sample from a subject with a chimeric protein comprising a surface (SU) of a non-foamy virus (non-FV) virus envelope protein bound or fused to a transmembrane (TM) of a FV envelope protein or a virus like particle comprising same; and determining an interaction between the sample and the chimeric protein. In some embodiments, arrays of different chimeric proteins comprising different POI may be employed.

Chimeric trimers are used, inter alia to screen antibodies present in a subject. Such methods may be useful prognostically and diagnostically.

The invention further provides a kit comprising a chimeric protein comprising a POI and a trimer forming portion of FV TM as described herein or a virus like particle comprising same. In some embodiments, the kit is conveniently used for (or is for use in) diagnosis or prognosis of a viral infection, or pathogen monitoring or serosurveillance kits, optionally including packaging, instructions and various other components such as buffers, substrates, antibodies or ligands, control antibodies or ligands, and detection reagents.

The invention further provides a kit comprising a chimeric protein wherein the protein comprises a surface (SU) of a non-foamy virus (non-FV) virus envelope protein bound or fused to a transmembrane (TM) of a FV envelope protein or a virus like particle comprising same. In some embodiments, the kit is conveniently used for or for use in diagnosing or prognosing a viral infection

In some embodiments, the present invention provides a method for producing an antibody comprising immunizing a subject animal or bird with a chimeric protein comprising a POI and a trimer forming portion of FV TM as described herein or a virus like particle comprising same. In some embodiments, the method includes purifying the antibody.

In some embodiments, the antibody binds to a trimeric antigen but not to a monomeric antigen and it is particularly useful for monitoring or quantifying viral particles, or detecting trimeric.

In some embodiments, the antigen is to a conserved region of the pathogen and therefore antibodies to the antigen detect a broad range of subspecies.

In other embodiments, the antigen is derived from a variant region.

In some embodiments, the present invention provides a method for producing an antibody comprising immunising a subject animal or bird with a chimeric protein comprising a surface protein (SU) of a non-foamy virus (non-FV) virus envelope protein bound or fused to a transmembrane protein (TM) of a FV envelope protein or a virus like particle comprising same. In some embodiments, the method includes purifying the antibody.

In some embodiments, the present invention provides a method for stimulating an immune response comprising administering a chimeric protein comprising a POI and a trimer forming portion of FV TM as described herein or a virus like particle comprising same to a subject animal or bird for a time and under conditions sufficient to engender a specific immune response to the POI or all or part of a pathogen comprising same. In some embodiments the immune response comprises the production of neutralizing antibodies to trimer or trimer and higher oligomeric forms of the POI. In some embodiments, the trimer forming portion comprises an N-terminal heptad repeat domain of FV TM and all or part of the cysteine-rich domain of FV TM.

In some embodiments, the present invention provides a method for stimulating a cellular immune response comprising immunizing a subject animal or bird with a chimeric protein comprising a surface protein (SU) of a non-foamy virus (non-FV) virus envelope protein bound or fused to a transmembrane protein (TM) of a FV envelope protein or a virus like particle comprising same.

In one particular embodiment the present invention provides an antibody determined by a chimeric protein of the present invention, such as trimeric HIV gp120, or an antigen-binding fragment or a chimeric, human or humanised form thereof.

The present antibodies bind specifically to the subject antigens.

The terms “antibody” and “antibodies” include polyclonal and monoclonal antibodies and all the various forms derived from monoclonal antibodies, including but not limited to full-length antibodies (e.g. having an intact Fc region), antigen-binding fragments, including for example, Fv, Fab, Fab′ and F(ab′)₂ fragments; and antibody-derived polypeptides produced using recombinant methods such as single chain antibodies. The terms “antibody” and “antibodies” as used herein also refer to human antibodies produced for example in transgenic animals or through phage display, as well as chimeric antibodies, human or humanized antibodies, primatised antibodies or deimmunised antibodies. It also includes other forms of antibodies that may be therapeutically acceptable and antigen-binding fragments thereof, for example single domain antibodies derived from cartilage marine animals or Camelidae, or from libraries based on such antibodies. The selection of fragmented or modified forms of the antibodies may also involve consideration of any affect the fragments or modified forms have on the half-lives of the antibody or fragment.

The term “monoclonal antibody” is used herein to refer to an antibody obtained from a population of substantially homogeneous antibodies. That is, the individual antibodies comprising the population are identical except for naturally occurring mutations that may be present in minor amounts. The term “monoclonal” as used herein indicates the character of the antibody as being obtained from a substantially homogeneous population of antibodies, and is not used to indicate that the antibody was produced by a particular method. For example, monoclonal antibodies in accordance with the present invention may be made by the hybridoma method described by Kohler and Milstein, Nature 256:495-499, 1975, or may be made by recombinant DNA methods (such as described in U.S. Pat. No. 4,816,567). Monoclonal antibodies may also be isolated from phage antibody libraries using the techniques described in Clackson et al., Nature 352:624-628, 1991 or Marks et al., J. Mol. Biol. 222:581-597, 1991.

The terms “effective amount” and “therapeutically effective amount” as used herein mean a sufficient amount of an agent which provides the desired therapeutic or physiological effect. Undesirable effects, e.g. side effects, may sometimes manifest along with the desired therapeutic effect; hence, a practitioner balances the potential benefits against the potential risks in determining an appropriate “effective amount”. The exact amount of agent required will vary from subject to subject, depending on the species, age and general condition of the subject, mode of administration and the like. Thus, it may not be possible to specify an exact “effective amount”. However, an appropriate “effective amount” in any individual case may be determined by one of ordinary skill in the art using routine experimentation. One of ordinary skill in the art would be able to determine the required amounts based on such factors as prior administration of agents, the subject's size, the severity of the subject's symptoms, viral load, and the particular composition or route of administration selected.

Insofar as one embodiment of the present invention relates to the use of neutralising antibodies to pathogens such as HIV the effective amount include from about 10 μg/kg body weight to 20 mg/kg body weight of antibody such as 10, 20, 30, 40, 50, 60, 70, 80, 90, 100 μg/kg body weight, 100, 200, 300, 400, 500, 600, 700, 800, 900, 1000 μg/kg body weight or 2, 3, 4, 5, 6, 7, 8, 9, 10, 11, 12, 13, 14, 15, 16, 17, 18, 19 or 20 mg/kg body weight.

Generally, the antibody is provided with a pharmaceutically or pharmacologically acceptable carrier, diluent or excipient.

Antibodies may be produced using recombinant methods (for example, in an E. coli expression system) well known in the art. In this approach, DNA encoding monoclonal antibodies, such as a murine monoclonal antibody produced by immunising a mouse with a chimeric trimeric protein of the present invention, may be isolated from the hybridoma cell lines, sequenced using standard procedures and optionally manipulated using recombinant DNA technology. For example, the DNA may be fused to another DNA of interest, or altered (such as by mutagenesis or other conventional techniques) to add, delete, or substitute one or more nucleic acid residues. The DNA may be placed into vectors which are then transfected or transformed into appropriate host cells using methods well known in the art (such as described in U.S. Pat. Nos. 4,399,216; 4,912,040; 4,740,461 and 4,959,455). The DNA isolated from the hybridoma cell lines may also be modified to change the character of the antibody produced by its expression.

Chimeric forms of murine monoclonal antibodies may be produced by replacing the nucleotides encoding selected murine heavy and light chain constant domains with nucleotides encoding human heavy and light chain constant domains, such as is described in U.S. Pat. No. 4,816,567 and by Morrison et al., Proc. Nat. Acad. Sci. 81:6851, 1984. The chimeric antibodies may then be produced in an appropriate cell line, such as a murine myeloma cell line, that has been transfected with modified DNA.

Among the antibodies contemplated by the present invention are chimeric antibodies that comprise the heavy and light chain variable regions of a murine monoclonal antibody fused to non-murine heavy and light chain antibody constant domains. In a particular embodiment, the non-murine heavy and light chain constant domains are human heavy and light chain antibody constant domains.

Antibodies contemplated in the present invention include humanised antibodies. In general, humanised antibodies are human antibodies (the recipient antibody) in which the complementarity determining (CDR) region residues have been replaced by CDR region residues from a non-human species (the donor antibody), such as from a mouse, rat, rabbit or non-human primate. In some cases, certain framework region (FR) residues of the human antibody may also be replaced by corresponding non-human residues, or the humanised antibodies may comprise residues which are not found in the recipient antibody or in the donor antibody. These modifications are made to enhance antibody performance and affinity. In general, the humanised antibody will comprise substantially all of at least one, and typically two, variable regions, in which all or substantially all of the CDR regions correspond to those of a non-human antibody, and all or substantially all of the FRs are those of a human antibody sequence. The humanised antibody may also optionally comprise at least a portion of an antibody constant region (Fc), typically that of a human antibody (Jones et al., Nature 321:522-525, 1986; Reichmann et al., Nature 332:323-329, 1988; Presta, Curr. Op. Struct. Biol. 2:593-596, 1992; Liu et al., Proc. Natl. Acad. Sci. USA 84:3439, 1987; Larrick et al., Bio/Technology 7:934, 1989; Winter & Harris, TIPS 14:139, 1993; Carter et al., Proc. Nat. Acad. Sci. 89:4285 1992). Similarly, to create a primatised antibody the murine CDR regions can be inserted into a primate framework using methods known in the art (see e.g. WO 93/02108 and WO 99/55369).

Alternatively, a humanised antibody may be created by a process of “veneering”. A statistical analysis of unique human and murine immunoglobulin heavy and light chain variable regions revealed that the precise patterns of exposed residues are different in human and murine antibodies, and most individual surface positions have a strong preference for a small number of different residues (see Padlan et al., Mol. Immunol. 28:489-498, 1991 and Pedersen et al., J. Mol. Biol. 235:959-973, 1994). Therefore, it is possible to reduce the immunogenicity of a non-human Fv by replacing exposed residues in its framework regions that differ from those usually found in human antibodies. Because protein antigenicity may be correlated with surface accessibility, replacement of the surface residues may be sufficient to render the mouse variable region “invisible” to the human immune system. This procedure of humanization is referred to as “veneering” because only the surface of the antibody is altered, the supporting residues remain undisturbed.

International publication No. WO 2004/006955 describes methods for humanizing antibodies, based on selecting variable region framework sequences from human antibody genes by comparing canonical CDR structure types for CDR sequences of the variable region of a non-human antibody to canonical CDR structure types for corresponding CDRs from a library of human antibody sequences, e.g. germline antibody gene segments. Human antibody variable regions having similar canonical CDR structure types to the non-human CDRs form a subset of member human antibody sequences from which to select human framework sequences. The subset members may be further ranked by amino acid similarity between the human and the non-human CDR sequences. In the method of WO 2004/006955, top ranking human sequences are selected to provide the framework sequences for constructing a chimeric antibody that functionally replaces human CDR sequences with the non-human CDR counterparts using the selected subset member human frameworks, thereby providing a humanised antibody of high affinity and low immunogenicity without need for comparing framework sequences between the non-human and human antibodies. The CDRs of a given antibody may be readily identified, for example using the system described by Kabat et al in Sequences of Proteins of Immunological Interest, 5th Ed., US Department of Health and Human Services, PHS, NIH, NIH Publication No. 91-3242, 1991).

In some embodiments, the antibodies are human monoclonal antibodies. Such human monoclonal antibodies directed against, for example HIV, can be generated using transgenic or transchromosomic mice carrying parts of the human immune system rather than the mouse system. These transgenic and transchromosomic mice include mice referred to herein as HuMAb mice and KM mice.

Alternative transgenic animal systems expressing human immunoglobulin genes are available in the art and can be used to raise antibodies. For example, an alternative transgenic system referred to as the Xenomouse (Abgenix, Inc.) can be used; such mice are described in, for example, U.S. Pat. Nos. 5,939,598; 6,075,181; 6,114,598; 6,150,584 and 6,162,963.

Moreover, alternative transchromosomic animal systems expressing human immunoglobulin genes are available in the art. For example, mice carrying both a human heavy chain transchromosome and a human light chain transchromosome, referred to as “TC mice” can be used; such mice are described in Tomizuka et al., Proc. Natl. Acad. Sci. USA 97:722-727, 2000.

Human monoclonal antibodies can also be prepared using phage display methods for screening libraries of human immunoglobulin genes. Such phage display methods for isolating human antibodies are established in the art. See for example: U.S. Pat. Nos. 5,223,409; 5,403,484; and 5,571,698; U.S. Pat. Nos. 5,427,908 and 5,580,717; U.S. Pat. Nos. 5,969,108 and 6,172,197 and U.S. Pat. Nos. 5,885,793; 6,521,404; 6,544,731; 6,555,313; 6,582,915 and 6,593,081.

Human monoclonal antibodies can also be prepared using SCID mice into which human immune cells have been reconstituted such that a human antibody response can be generated upon immunization. Such mice are described in, for example, U.S. Pat. Nos. 5,476,996 and 5,698,767.

A number of recombinant methods have now been developed for producing antigen-binding fragments of antibodies directly in recombinant host cells. For example, Fab′-SH fragments can be directly recovered from E. coli and chemically coupled to form F(ab′)₂ fragments (Carter et al., Bio/Technology 10:163-167, 1992). F(ab′)₂ fragments can also be formed using the leucine zipper GCN4 to promote assembly of the F(ab′)₂ molecule. Fv, Fab or F(ab′)₂ fragments can also be isolated directly from recombinant host cell cultures. A number of recombinant methods have been developed for the production of single chain antibodies including those described in U.S. Pat. No. 4,946,778; Bird, Science 242:423, 1988, Huston et al., Proc. Natl. Acad. Sci. USA 85:5879, 1988 and Ward et al., Nature 334:544, 1989. Single chain antibodies may be formed by linking heavy (V_{H}) and light (V_{L}) chain variable region (Fv region) fragments via an short peptide linker to provide a single polypeptide chain (scFvs). The scFvs may also form dimers or trimers, depending on the length of a peptide linker between the two variable regions (Kortt et al., Protein Engineering 10:423, 1997). Phage display is another well known recombinant method for producing the antigen-binding fragments of the present invention.

The antigen-binding fragments of the antibody may be screened for desired properties. The assays described herein provide the means to identify antibodies and other ligands that reduce infectivity.

Mammalian cell lines available as hosts for expression are well known in the art and include many immortalized cell lines available from the American Type Culture Collection (ATCC). These include, inter alia, Chinese hamster ovary (CHO) cells, NSO, SP2 cells, HeLa cells, baby hamster kidney (BHK) cells, monkey kidney cells (COS), human hepatocellular carcinoma cells (e.g. Hep G2), A549 cells, 3T3 cells, and a number of other cell lines. Other cell lines that may be used are insect cell lines, such as Sf9. cells, amphibian cells, bacterial cells, plant cells and fungal cells. When recombinant expression vectors encoding the heavy chain or antigen-binding portion thereof, the light chain and/or antigen-binding portion thereof are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods. Further, expression of antibodies of the invention from host cell lines can be enhanced using a number of known techniques.

Techniques are also known for deriving an antibody of a different subclass or isotype from an antibody of interest, i.e. subclass switching. Thus, IgG1 or IgG4 monoclonal antibodies may be derived from an IgM monoclonal antibody, for example, and vice versa. Such techniques allow the preparation of new antibodies that possess the antigen-binding properties of a given antibody (the parent antibody), but also exhibit biological properties associated with an antibody isotype or subclass different from that of the parent antibody. Recombinant DNA techniques may be employed. Cloned DNA encoding particular antibody polypeptides may be employed in such procedures, e.g. DNA encoding the constant region of an antibody of the desired isotype.

Vectors available for cloning and expression in host cell lines are well known in the art, and include but are not limited to vectors for cloning and expression in mammalian cell lines, vectors for cloning and expression in bacterial cell lines, vectors for cloning and expression in phage and vectors for cloning and expression insect cell lines. The antibodies can be recovered using standard protein purification methods.

Chemical analogs contemplated herein include, but are not limited to, modifications of side chains, incorporation of unnatural amino acids and/or their derivatives during peptide, polypeptide or protein synthesis and the use of crosslinkers and other methods which impose conformational constraints on the proteinaceous molecule or their analogs.

The invention provides a method for producing an antibody comprising immunising a non-human animal or screening expression products of a library of human immunoglobulin genes with a chimeric protein as described herein, a viral-like particle comprising same or a nucleic acid encoding same and isolating an antibody that binds specifically to the polypeptide or peptide of interest or to all or part of a pathogen comprising same.

In another embodiment, the invention provides an antibody produced by the methods described herein using a subject chimeric protein, or an antigen-binding fragment or a chimeric, human or humanised form thereof. The antibody is preferable monoclonal rather than polyclonal and is preferably chimeric, humanised, deimmunised or is a human antibody.

In one embodiment, the present invention contemplates a use of a chimeric protein as described herein or a virus like particle comprising same in the manufacture of a medicament for the treatment or prophylaxis of a condition such as a viral infection or an infection with a pathogen, or cancer.

The terms “treatment” or “prophylaxis” or “therapy” are used interchangeably in their broadest context and include any measurable or statistically significant amelioration in at least some subjects in one or more symptoms of a condition to be treated or in the risk of developing a particular condition. Prophylaxis may be considered as reducing the severity or onset of a condition or signs of a condition. Treatment may also reduce the severity of existing conditions.

In one embodiment, the present invention contemplates a use of a chimeric protein as described herein or a virus like particle comprising same in the manufacture of a medicament for the treatment or prophylaxis of a disease or condition, such as an infection with a virus, bacteria, parasite or a condition such as a cancer.

A non-exhaustive list of suitable diseases includes: tuberculosis, HIV, malaria. H. pylori, influenza, hepatitis, CMV, human papilloma virus (HPV), herpes virus-induced diseases and other viral infections, leprosy, non-malarial protozoan parasites such as toxoplasma, and various malignancies such as tumours and/or cancers, infectious disease caused by protozoans: malaria, particularly Plasmodium falciparum and P. vivax, toxoplasma, Theileria parva, Trypanosoma cruzi, by mycobacteria such as tuberculosis and leprosy, bacteria such as Chlamydia pneumoniae and Helicobacter pylori, by viruses such as HIV, EBV, CMV, HBV, HCV, HPV, HSV, RSV, influenza virus and various malignacies such as renal, colorectal, lung, skin (melanoma), liver, ovary, testis, pancreas, uterus, prostate, stomach, head and neck, cervix, breast cancer and various lymphomas, as well as HIV/AIDS, hepatitis B, hepatitis C, malaria, tuberculosis, HPV infection and disease, HSV infection and disease, CMV infection and disease, EBV infection and disease, leishmaniasis, listeriosis, theileria, HTLV infection and disease, pneumococcal disease, staphylococcal disease, lung cancer, breast cancer, colon cancer, melanoma, myeloma, lymphoma, renal cell carcinoma.

For the avoidance of doubt, in some embodiments, VLPs are contemplated comprising chimeric proteins comprising TM FV bound to two or more different non-foamy virus virus envelope proteins.

By “co-administered” is meant simultaneous administration in the same formulation or in two different formulations via the same or different routes or sequential administration by the same or different routes. For example, the HA1gp47 trimers may be co-administered together with other antigens such as neuraminidase or matrix protein of influenza in order to enhance its effects. In another example, soluble trimers are co-administered with VLPs. By “sequential” administration is meant a time difference of from seconds, minutes, hours or days between the administration of the two types of molecules. These molecules may be administered in any order.

In another embodiment, the invention provides a chimeric protein as described herein or a virus like particle comprising same for use in the treatment or prophylaxis of a viral infection.

In another embodiment, the invention provides a chimeric protein comprising a surface (SU) of a non-foamy virus (non-FV) virus envelope protein bound or fused to a transmembrane (TM) of a FV envelope protein or a virus like particle comprising same for use in the treatment or prophylaxis of a viral infection. Illustrative viral infections are HIV, SARS, RSV, Dengue virus and Influenza.

In a similar embodiment, the invention provides a method for treatment or prophylaxis of a viral infection the method comprising administering a chimeric protein comprising a surface (SU) of a non-foamy virus (non-FV) virus envelope protein bound or fused to a transmembrane (TM) of a FV envelope protein or a virus like particle comprising to a subject in need thereof.

In some embodiments, the chimeric proteins are delivered to a subject, animal or bird in genetic form.

In some embodiments, a chimeric polypeptide or VLP comprising same or nucleic acid encoding same or an antibody determined thereby are provided in an “effective amount” sufficient to treat or reduce a viral infection.

The chimeric proteins, their encoding nucleic acid molecules and VLPs comprising same are contemplated for use in the manufacture of compositions including pharmaceutical compositions. Illustrative compositions are those developed for immunisation.

In some embodiments, the invention provides pharmaceutical compositions comprising the isolated nucleic acid molecule. In some embodiments, pharmaceutical compositions are formulated with a pharmaceutically acceptable carrier and/or diluent.

In other embodiments, the present invention provides a pharmaceutical composition comprising a chimeric protein as described herein including a chimeric protein comprising a surface protein (SU) of a non-foamy virus (non-FV) virus envelope protein bound or fused to a transmembrane protein (TM) of a FV envelope protein or a virus like particle comprising same.

The pharmaceutical composition comprising the chimeric protein, including VLPs comprising same, is contemplated to exhibit therapeutic activity when administered in an amount which depends on the particular case. The variation depends, for example, on the human or animal and the agent chosen. A broad range of doses may be applicable. Considering a subject, for example, from about 0.1 μg to 1 μg (i.e., including 0.1 μg, 0.2 μg, 0.3 μg, 0.4 μg, 0.5 μg, 0.6 μg, 0.7 μg, 0.8 μg and 0.9 μg) 0.5 μg to 50 μg, 1 μg to 10 μg, 2 μg to 200 μg, 0.1 mg to 1.0 mg (i.e., including 0.1 mg, 0.2 mg, 0.3 mg, 0.4 mg, 0.5 mg, 0.6 mg, 0.7 mg, 0.8 mg and 0.9 mg), from about 15 mg to 35 mg, about 1 mg to 30 mg or from 5 to 50 mg, or from 10 mg to 100 mg of agent may be administered per kilogram of body weight per day or per every other day or per week or per month. Therapeutic including prophylactic compositions may be administered at a dosage of about 0.1 to 20 mg/kg however dosages above or below this amount are contemplated in the ranges set out above. Dosage regimes may be adjusted to provide the optimum therapeutic response. For example, several divided doses may be administered daily, weekly, monthly or other suitable time intervals or the dose may be proportionally reduced as indicated by the exigencies of the situation. It is also possible to administer compositions in sustained release formulations.

Administration is generally for a time and under conditions sufficient to treat or prevent a viral infection. The agents may be administered in a convenient manner such as by the oral, intravenous (where water soluble), intraperitoneal', intramuscular, subcutaneous, intradermal, intrathecal or suppository routes or implanting (e.g. using slow release molecules). Administration may be systemic or local, although systemic is more convenient. References to systemic include intravenous, intraperitoneal, subcutaneous injection, infusion as well as administration via oral, rectal, vaginal and nasal routes or via inhalation which is advantageous. Other contemplated routes of administration are by patch, cellular transfer, implant, sublingually, intraocularly, topically or transdermally. Depending upon the severity or stage of disease and integrity of the blood brain barrier, suitable compositions are required to cross the blood brain barrier.

Pharmaceutical compositions are conveniently prepared according to conventional pharmaceutical compounding techniques. See, for example, Remington's Pharmaceutical Sciences, 18th Ed., Mack Publishing, Company, Easton, Pa., U.S.A., 1990. The composition may contain the active agent or pharmaceutically acceptable salts of the active agent. These compositions may comprise, in addition to one of the active substances, a pharmaceutically acceptable excipient, carrier, buffer, stabilizer or other materials well known in the art. Such materials should be non-toxic and should not interfere with the efficacy of the active ingredient. The carrier may take a wide variety of forms depending on the form of preparation desired for administration, e.g. intravenous, oral or parenteral.

A “pharmaceutically acceptable carrier” and/or a diluent is a pharmaceutical vehicle comprised of a material that is not otherwise undesirable i.e., it is unlikely to cause a substantial adverse reaction by itself or with the active agent. Carriers may include all solvents, dispersion media, coatings, antibacterial and antifungal agents, agents for adjusting tonicity, increasing or decreasing absorption or clearance rates, buffers for maintaining pH, chelating agents, membrane or barrier crossing agents. A pharmaceutically acceptable salt is a salt that is not otherwise undesirable. The agent or composition comprising the agent may be administered in the form of pharmaceutically acceptable non-toxic salts, such as acid addition salts or metal complexes,

For oral administration, the compounds can be formulated into solid or liquid preparations such as capsules, pills, tablets, lozenges, powders, suspensions or emulsions. In preparing the compositions in oral dosage form, any of the usual pharmaceutical media may be employed, such as, for example, water, glycols, oils, alcohols, flavoring agents, preservatives, coloring agents, suspending agents, and the like in the case of oral liquid preparations (such as, for example, suspensions, elixirs and solutions); or carriers such as starches, sugars, diluents, granulating agents, lubricants, binders, disintegrating agents and the like in the case of oral solid preparations (such as, for example, powders, capsules and tablets). Because of their ease in administration, tablets and capsules represent the most advantageous oral dosage unit form, in which case solid pharmaceutical carriers are obviously employed. Tablet may contain a binder such as tragacanth, corn starch or gelatin; a disintegrating agent, such as alginic acid; and a lubricant, such as magnesium stearate. If desired, tablets may be sugar-coated or enteric-coated by standard techniques. The active agent can be encapsulated to make it stable to passage through the gastrointestinal tract. See for example, International Patent Publication No. WO 96/11698.

For parenteral administration, the composition may be dissolved in a carrier and administered as a solution or a suspension. When the agents are administered intrathecally, they may also be dissolved in cerebrospinal fluid. For transmucosal or transdermal (including patch) delivery, appropriate penetrants known in the art are used for delivering the antagonist. For inhalation, delivery uses any convenient system such as dry powder aerosol, liquid delivery systems, air jet nebulizers, propellant systems. For example, the formulation can be administered in the form of an aerosol or mist. The agents may also be delivered in a sustained delivery or sustained release format. For example, biodegradable microspheres or capsules or other polymer configurations capable of sustained delivery can be included in the formulation. Formulations can be modified to alter pharmacokinetics and biodistribution. For a general discussion of pharmacokinetics, see, e.g., Remington's. In some embodiments the formulations may be incorporated in lipid monolayers or bilayers such as liposomes or micelles. Targeting therapies known in the art may be used to deliver the agents more specifically to certain types of cells or tissues.

The actual amount of active agent administered and the rate and time-course of administration will depend on the nature and severity of the disease. Prescription of treatment, e.g. decisions on dosage, timing, etc. is within the responsibility of general practitioners or specialists and typically takes into account the condition of the individual patient, the site of delivery, the method of administration and other factors known to practitioners. Examples of techniques and protocols can be found in Remington's Pharmaceutical Sciences (supra).

Sustained-release preparations that may be prepared are particularly convenient for inducing immune responses. Examples of sustained-release preparations include semipermeable matrices of solid hydrophobic polymers containing the antagonist, which matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides, copolymers of L-glutamic acid and ethyl-L-glutamate, non-degradable ethylene-vinyl acetate, degradable lactic acid-glycolic acid copolymers, and poly-D-(−)-3-hydroxybutyric acid. While polymers such as ethylene-vinyl acetate and lactic acid-glycolic acid enable release of molecules for over 100 days, certain hydrogels release proteins for shorter time periods. Liposomes may be used which are of the small (about 200-800 Angstroms) unilamellar type in which the lipid content is greater than about 30% cholesterol, the selected proportion being adjusted for the optimal therapy.

Stabilization of proteins may be achieved by modifying sulfhydryl residues, lyophilizing from acidic solutions, controlling moisture content, using appropriate additives, and developing specific polymer matrix compositions. The in vivo half life of proteins may be extended using techniques known in the art, including, for example, by the attachment of other elements such as polyethyleneglycol (PEG) groups.

Prime-boost immunisation strategies as disclosed in the art are clearly contemplated. See for example International Publication No. WO/2003/047617. Thus, compositions may be in the form of a vaccine, priming or boosting agent.

Instead of administering these agents directly, they could be produced in the target cell, e.g. in a viral vector such as described below or in a cell based delivery system such as described in U.S. Pat. No. 5,550,050 and International Patent Publication Nos. WO 92/19195, WO 94/25503, WO 95/01203, WO 95/05452, WO 96/02286, WO 96/02646, WO 96/40871, WO 96/40959 and WO 97/12635. The vector could be targeted to the target cells. The cell based delivery system is designed to be implanted in a patient's body at the desired target site and contains a coding sequence for the target agent. Alternatively, the agent could be administered in a precursor form for conversion to the active form by an activating agent produced in, or targeted to, the cells to be treated. See, for example, European Patent Application No. 0 425 731A and International Patent Publication No. WO 90/07936.

Vaccine composition may therefore alternatively comprise nucleic acid molecules or constructs encoding the subject chimeric proteins or VLPs comprising same.

Gene transfer systems known in the art may be useful in the practice of genetic manipulation. These include viral and non-viral transfer methods. A number of viruses have been used as gene transfer vectors or as the basis for preparing gene transfer vectors, including papovaviruses (e.g. SV40, Madzak et al., J. Gen. Virol. 73: 1533-1536, 1992), adenovirus (Berkner, Curr. Top. Microbiol. Immunol. 158: 39-66, 1992; Berkner et al., BioTechniques 6; 616-629, 1988; Gorziglia and Kapikian, J. Virol. 66: 4407-4412, 1992; Quantin et al., Proc. Natl. Acad. Sci. USA 89: 2581-2584, 1992; Rosenfeld et al., Cell 68: 143-155, 1992; Wilkinson et al., Nucleic Acids Res. 20: 2233-2239, 1992; Stratford-Perricaudet et al., Hum. Gene Ther. 1: 241-256, 1990; Schneider et al., Nature Genetics 18: 180-183, 1998), vaccinia virus (Moss, Curr. Top. Microbiol. Immunol. 158: 25-38, 1992; Moss, Proc. Natl. Acad. Sci. USA 93: 11341-11348, 1996), adeno-associated virus (Muzyczka, Curr. Top. Microbiol. Immunol. 158: 97-129, 1992; Ohi et al., Gene 89: 279-282, 1990; Russell and Hirata, Nature Genetics 18: 323-328, 1998), herpesviruses including HSV and EBV (Margolskee, Curr. Top., Microbiol. Immunol. 158: 67-95, 1992; Johnson et al., J. Virol. 66: 2952-2965, 1992; Fink et al., Hum. Gene Ther. 3: 11-19, 1992; Breakefield and Geller, Mol. Neurobiol. 1: 339-371, 1987; Freese et al., Biochem. Pharmacol. 40: 2189-2199, 1990; Fink et al., Ann. Rev. Neurosci. 19: 265-287, 1996), lentiviruses (Naldini et al., Science 272: 263-267, 1996), Sindbis and Semliki Forest virus (Berglund et al., Biotechnology 11: 916-920, 1993) and retroviruses of avian (Bandyopadhyay and Temin, Mol. Cell. Biol. 4: 749-754, 1984; Petropoulos et al., J. Viol. 66: 3391-3397, 1992], murine [Miller, Curr. Top. Microbiol. Immunol. 158: 1-24, 1992; Miller et al., Mol. Cell. Biol. 5: 431-437, 1985; Sorge et al., Mol. Cell. Biol. 4: 1730-1737, 1984; Mann and Baltimore, J. Virol. 54: 401-407, 1985; Miller et al., J. Virol. 62: 4337-4345, 1988] and human [Shimada et al., J. Clin. Invest. 88: 1043-1047, 1991; Helseth et al., J. Virol. 64: 2416-2420, 1990; Page et al., J. Virol. 64: 5270-5276, 1990; Buchschacher and Panganiban, J. Virol. 66: 2731-2739, 1982] origin.

Non-viral gene transfer methods are known in the art such as chemical techniques including calcium phosphate co-precipitation, mechanical techniques, for example, microinjection, membrane fusion-mediated transfer via liposomes and direct DNA uptake and receptor-mediated DNA transfer. Naked DNA (or RNA) may be linear or circular (for example, a plasmid). It may be provided in a carrier such as a liposome or in a free form. Viral-mediated gene transfer can be combined with direct in vivo gene transfer using liposome delivery, allowing one to direct the viral vectors to particular cells. Alternatively, the retroviral vector producer cell line can be injected into particular tissue. Injection of producer cells would then provide a continuous source of vector particles.

In an approach which combines biological and physical gene transfer methods, plasmid DNA of any size is combined with a polylysine-conjugated antibody specific to the adenovirus hexon protein and the resulting complex is bound to an adenovirus vector. The trimolecular complex is then used to infect cells. The adenovirus vector permits efficient binding, internalization and degradation of the endosome before the coupled DNA is damaged. For other techniques for the delivery of adenovirus based vectors, see U.S. Pat. No. 5,691,198.

Liposome/DNA complexes have been shown to be capable of mediating direct in vivo gene transfer. While in standard liposome preparations the gene transfer process is non-specific, localised in vivo uptake and expression have been reported in tumor deposits, for example, following direct in situ administration.

By “nucleic acid immunization” is meant the introduction of a nucleic acid molecule encoding one or more selected chimeric proteins into a host cell, for in vivo expression of an antigen, antigens, an epitope, or epitopes. The nucleic acid molecule can be introduced directly into a recipient subject, such as by injection, inhalation, oral, intranasal and mucosal administration, or the like, or can be introduced ex vivo, into cells which have been removed from the host. In the latter case, the transformed cells are reintroduced into the subject where an immune response can be mounted against the antigen encoded by the nucleic acid molecule.

“Gene transfer” or “gene delivery” refers to methods or systems for reliably inserting DNA or RNA of interest into a host cell. Such methods can result in transient expression of non-integrated transferred DNA, extrachromosomal replication and expression of transferred replicons (e.g., episomes), or integration of transferred genetic material into the genomic DNA of host cells. Gene delivery expression vectors include, but are not limited to, vectors derived from bacterial plasmid vectors, viral vectors, non-viral vectors, alphaviruses, pox viruses and vaccinia viruses. When used for immunization, such gene delivery expression vectors may be referred to as vaccines or vaccine vectors.

The term “isolated” means material that is substantially or essentially free from components that normally accompany it in its native state. For example, an “isolated nucleic acid molecule”, as used herein, refers to a nucleic acid or polynucleotide, isolated from the sequences which flank it in a naturally-occurring state, e.g., a DNA fragment which has been removed from the sequences that are normally adjacent to the fragment. Alternatively, an “isolated chimeric protein” or an “isolated polypeptide” and the like, as used herein, refer to in vitro isolation and/or purification of a protein from its natural cellular environment, and from association with other components of the cell. Without limitation, an isolated nucleic acid, polynucleotide, peptide, or polypeptide can refer to a native sequence that is isolated by purification or to a sequence that is produced by recombinant or synthetic means.

By “effective amount,” in the context of treatment or prophylaxis of an infection or condition is meant the administration of that amount of active to a subject, either in a single dose or as part of a series or slow release system that is effective for producing a therapeutic effect, in some subjects. The effective amount will vary depending upon the health and physical condition of the subject and the taxonomic group of individual to be treated, the formulation of the composition, the assessment of the medical situation, and other relevant factors. It is expected that the amount will fall in a relatively broad range that can be determined through routine trials.

By “biologically active portion” or “biologically active part” or “functional part or portion” is meant a portion of a full-length POI such as HA or gp120 or a portion of FV TM polypeptide or a portion of a herein described chimeric polypeptide which portion retains the activity of the full length molecule at least in so far as it retains the structural and functional abilities to form dimers, trimers or other oligomers. As used herein, the term “biologically active portion” includes deletion mutants and peptides, for example of at least about 8, 9, 10, 11, 12, 13, 14, 15, 16, 17, 18, 19, 20, 21, 22, 23, 24, 25, 30, 40, 50, 60, 70, 80, 90, 100, 120, 150, 300, 350 contiguous amino acids (and every integer in between), which retains activity. One example of a biologically active portion of FV TM (gp47) is a form of the polypeptide without a fusion domain or without a cytoplasmic domain. Another important part is the long heptad repeat region of gp47 which is approximately 74 amino acids in length. Portions of this type may be obtained through the application of standard recombinant nucleic acid techniques or synthesised using conventional or state of the art liquid or solid phase synthesis techniques. For example, reference may be made to solution synthesis or solid phase synthesis as described, for example, in Chapter 9 entitled “Peptide Synthesis” by Atherton and Shephard which is included in a publication entitled “Synthetic Vaccines” edited by Nicholson and published by Blackwell Scientific Publications. Alternatively, peptides can be produced by digestion of a peptide or polypeptide of the invention with proteinases such as endoLys-C, endoArg-C, endoGlu-C and staphylococcus V8-protease. The digested fragments can be purified by, for example, high performance liquid chromatographic (HPLC) techniques. Recombinant nucleic acid techniques can also be used to produce such portions. The biological activities of portions are tested in vivo and/or in vitro.

By “vector” is meant a polynucleotide molecule, suitably a DNA molecule derived, for example, from a plasmid, bacteriophage, yeast, virus, mammal, avian, reptile or fish into which a polynucleotide can be inserted or cloned. A vector preferably contains one or more unique restriction sites and can be capable of autonomous replication in a defined host cell including a target cell or tissue or a progenitor cell or tissue thereof, or be integrable with the genome of the defined host such that the cloned sequence is reproducible. Accordingly, the vector can be an autonomously replicating vector, i.e., a vector that exists as an extrachromosomal entity, the replication of which is independent of chromosomal replication, e.g., a linear or closed circular plasmid, an extrachromosomal element, a minichromosome, or an artificial chromosome. The vector can contain any means for assuring self-replication. Alternatively, the vector can be one which, when introduced into the host cell, is integrated into the genome and replicated together with the chromosome(s) into which it has been integrated. A vector system can comprise a single vector or plasmid, two or more vectors or plasmids, which together contain the total DNA to be introduced into the genome of the host cell, or a transposon. The choice of the vector will typically depend on the compatibility of the vector with the host cell into which the vector is to be introduced. The vector can also include a selection marker such as an antibiotic resistance gene that can be used for selection of suitable transformants. Examples of such resistance genes are known to those of skill in the art.

By “derivative” is meant a polypeptide that has been derived from the basic sequence by modification, for example by conjugation or complexing with other chemical moieties or by post-translational modification techniques as would be understood in the art. The term “derivative” also includes within its scope alterations that have been made to a FV TM, POI or POI-FV TM polypeptide including additions, or deletions that provide for functionally equivalent molecules.

A “part” or “portion” of a polynucleotide is defined as having a minimal size of at least about 10 nucleotides or preferably about 13 nucleotides or more preferably at least about 20 nucleotides and may have a minimal size of at least about 35 nucleotides. This definition includes all sizes in the range of 10-35 nucleotides including 10, 11, 12, 13, 14, 15, 16, 17, 18, 19, 20, 21, 22, 23, 24, 25, 26, 27, 28, 29, 30, 31, 32, 33, 34 or 35 nucleotides as well as greater than 35 nucleotides including 50, 100, 300, 500, 600 nucleotides or nucleic acid molecules having any number of nucleotides within these values.

Reference to functional variants include those that are distinguished from a naturally-occurring form or from forms presented herein by the addition, deletion and/or substitution of at least one amino acid residue. Thus, variants include proteins derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Variant proteins encompassed by the present invention are biologically active, that is, they continue to possess the desired biological activity of the native protein (e.g., immunogenicity or ability to form trimers with FVTM etc). Variants may result from, for example, genetic polymorphism or from human manipulation. Biologically active variants of a viral polypeptide will typically have at least 40%, 50%, 60%, 70%, generally at least 75%, 80%, 85%, preferably about 90% to 95% or more, and more preferably about 98% or more sequence similarity or identity with the amino acid sequence for the protein described herein as determined by sequence alignment programs described elsewhere herein using default parameters. A biologically active variant of a chimeric polypeptide may differ from that polypeptide generally by as much 100, 50 or 20 amino acid residues or suitably by as few as 1-15 amino acid residues, as few as 1-10, such as 6-10, as few as 5, as few as 4, 3, 2, or even 1 amino acid residue.

A variant polypeptide may be altered in various ways including amino acid substitutions, deletions, truncations, and insertions. Methods for such manipulations are generally known in the art. For example, amino acid sequence variants of a chimeric polypeptide can be prepared by mutations in the DNA. Methods for mutagenesis and nucleotide sequence alterations are well known in the art. See, for example, Kunkel, Proc. Natl. Acad. Sci. USA, 82: 488-492, 1985; Kunkel et al., Methods in Enzymol., 154: 367-382, 1987; U.S. Pat. No. 4,873,192; Watson et al., Molecular Biology of the Gene, Fourth Edition, Benjamin/Cummings, Menlo Park, Calif., 1987) and the references cited therein. Guidance as to appropriate amino acid substitutions that do not affect biological activity of the protein of interest may be found in the model of Dayhoff et al., Atlas of Protein Sequence and Structure, Natl. Biomed. Res. Found., Washington, D.C., 1978. Methods for screening gene products of combinatorial libraries made by point mutations or truncation, and for screening cDNA libraries for gene products having a selected property are known in the art. Such methods are adaptable for rapid screening of the gene libraries generated by combinatorial mutagenesis of chimeric polypeptides. Recursive ensemble mutagenesis (REM), a technique which enhances the frequency of functional mutants in the libraries, can be used in combination with the screening assays to identify chimeric polypeptide variants (Arkin and Yourvan, Proc. Natl. Acad. Sci. USA, 89: 7811-7815, 1992; Delgrave et al., Protein Engineering, 6: 327-331, 1993). Conservative substitutions, such as exchanging one amino acid with another having similar properties, are desirable as discussed in more detail below.

Variant chimeric polypeptides may contain conservative amino acid substitutions at various locations along their sequence, as compared to the reference amino acid sequence. A “conservative amino acid substitution” is one in which the amino acid residue is replaced with an amino acid residue having a similar side chain. Families of amino acid residues having similar side chains have been defined in the art, which can be generally sub-classified as follows:

Acidic: The residue has a negative charge due to loss of H ion at physiological pH and the residue is attracted by aqueous solution so as to seek the surface positions in the conformation of a peptide in which it is contained when the peptide is in aqueous medium at physiological pH. Amino acids having an acidic side chain include glutamic acid and aspartic acid.

Basic: The residue has a positive charge due to association with H ion at physiological pH or within one or two pH units thereof (e.g., histidine) and the residue is attracted by aqueous solution so as to seek the surface positions in the conformation of a peptide in which it is contained when the peptide is in aqueous medium at physiological pH. Amino acids having a basic side chain include arginine, lysine and histidine.

Charged: The residues are charged at physiological pH and, therefore, include amino acids having acidic or basic side chains (i.e., glutamic acid, aspartic acid, arginine, lysine and histidine).

Hydrophobic: The residues are not charged at physiological pH and the residue is repelled by aqueous solution so as to seek the inner positions in the conformation of a peptide in which it is contained when the peptide is in aqueous medium. Amino acids having a hydrophobic side chain include tyrosine, valine, isoleucine, leucine, methionine, phenylalanine and tryptophan.

Neutral/polar: The residues are not charged at physiological pH, but the residue is not sufficiently repelled by aqueous solutions so that it would seek inner positions in the conformation of a peptide in which it is contained when the peptide is in aqueous medium. Amino acids having a neutral/polar side chain include asparagine, glutamine, cysteine, histidine, serine and threonine.

This description also characterizes certain amino acids as “small” since their side chains are not sufficiently large, even if polar groups are lacking, to confer hydrophobicity. With the exception of proline, “small” amino acids are those with four carbons or less when at least one polar group is on the side chain and three carbons or less when not. Amino acids having a small side chain include glycine, serine, alanine and threonine. The gene-encoded secondary amino acid proline is a special case due to its known effects on the secondary conformation of peptide chains. The structure of proline differs from all the other naturally-occurring amino acids in that its side chain is bonded to the nitrogen of the α-amino group, as well as the α-carbon. Several amino acid similarity matrices (e.g., PAM120 matrix and PAM250 matrix as disclosed for example by Dayhoff et al. 1978, (supra), A model of evolutionary change in proteins. Matrices for determining distance relationships In M. O. Dayhoff, (ed.), Atlas of protein sequence and structure, Vol. 5, pp. 345-358, National Biomedical Research Foundation, Washington D.C.; and by Gonnet et al., Science, 256(5062): 1443-1445, 1992), however, include proline in the same group as glycine, serine, alanine and threonine. Accordingly, for the purposes of the present invention, proline is classified as a “small” amino acid.

The degree of attraction or repulsion required for classification as polar or nonpolar is arbitrary and, therefore, amino acids specifically contemplated by the invention have been classified as one or the other. Most amino acids not specifically named can be classified on the basis of known behavior.

Amino acid residues can be further sub-classified as cyclic or noncyclic, and aromatic or nonaromatic, self-explanatory classifications with respect to the side-chain substituent groups of the residues, and as small or large. The residue is considered small if it contains a total of four carbon atoms or less, inclusive of the carboxylcarbon, provided an additional polar substituent is present; three or less if not. Small residues are, of course, always nonaromatic. Dependent on their structural properties, amino acid residues may fall in two or more classes. For the naturally-occurring protein amino acids, sub-classification according to this scheme is presented in the Table 3.

Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. For example, it is reasonable to expect that replacement of a leucine with an isoleucine or valine, an aspartate with a glutamate, a threonine with a serine, or a similar replacement of an amino acid with a structurally related amino acid will not have a major effect on the properties of the resulting variant polypeptide. Whether an amino acid change results in a functional chimeric polypeptide can readily be determined by assaying its activity. Conservative substitutions are shown in Table 4 (below) under the heading of exemplary substitutions. More preferred substitutions are shown under the heading of preferred substitutions. Amino acid substitutions falling within the scope of the invention, are, in general, accomplished by selecting substitutions that do not differ significantly in their effect on maintaining (a) the structure of the peptide backbone in the area of the substitution, (b) the charge or hydrophobicity of the molecule at the target site, or (c) the bulk of the side chain. After the substitutions are introduced, the variants are screened for biological activity

Alternatively, similar amino acids for making conservative substitutions can be grouped into three categories based on the identity of the side chains. The first group includes glutamic acid, aspartic acid, arginine, lysine, histidine, which all have charged side chains; the second group includes glycine, serine, threonine, cysteine, tyrosine, glutamine, asparagine; and the third group includes leucine, isoleucine, valine, alanine, proline, phenylalanine, tryptophan, methionine, as described in Zubay, G., Biochemistry, third edition, Wm.C. Brown Publishers (1993).

Thus, a predicted non-essential amino acid residue in a chimeric polypeptide is typically replaced with another amino acid residue from the same side chain family. Alternatively, mutations can be introduced randomly along all or part of a chimeric polynucleotide coding sequence, such as by saturation mutagenesis, and the resultant mutants can be screened for an activity of the parent polypeptide to identify mutants which retain that activity. Following mutagenesis of the coding sequences, the encoded peptide can be expressed recombinantly and the activity of the peptide can be determined.

Accordingly, the present invention also contemplates variants of the chimeric polypeptide sequences provided herein or their biologically-active fragments, wherein the variants are distinguished from the provided sequences by the addition, deletion, or substitution of one or more amino acid residues. In general, variants will display at least about 30, 40, 50, 55, 60, 65, 70, 75, 80, 85, 90, 91, 92, 93, 94, 95, 96, 97, 98, 99% similarity to a reference chimeric polypeptide sequence as, for example, set forth in any one of SEQ ID NOs: 10, 12, 14, 22, 26 and 28. Desirably, variants will have at least 30, 40, 50, 55, 60, 65, 70, 75, 80, 85, 90, 91, 92, 93, 94, 95, 96, 97, 98, 99% sequence identity to a parent chimeric polypeptide sequence as, for example, set forth in any one of SEQ ID NOs: 10, 12, 14, 22, 26 and 28. Moreover, sequences differing from the disclosed sequences by the addition, deletion, or substitution of 1, 2, 3, 4, 5, 6, 7, 8, 9, 10, 11, 12, 13, 14, 15, 16, 17, 18, 19, 20, 30, 40, 50, 60, 70, 80, 90, 100 or more amino acids but which retain the biological activity of the parent chimeric polypeptide are contemplated. Variant chimeric polypeptides also include polypeptides that are encoded by polynucleotides that hybridize under stringency conditions as defined herein, especially high stringency conditions, to disclosed polynucleotide sequences, or the non-coding strand thereof. Illustrative polynucleotide sequences are set forth in any one of SEQ ID NOs: 2, 7, 9, 11, 13, 15, 16, 17, 18, 21, 23, and 27.

In some embodiments, variant polypeptides differ from a FV TM or POI-FV TM sequence by at least one but by less than 50, 40, 30, 20, 15, 10, 8, 6, 5, 4, 3 or 2 amino acid residue(s). In another, variant polypeptides differ from the recited sequence by at least 1% but less than 20%, 15%, 10% or 5% of the residues. (If this comparison requires alignment the sequences should be aligned for maximum similarity. “Looped” out sequences from deletions or insertions, or mismatches, are considered differences.) The differences are, suitably, differences or changes at a non-essential residue or a conservative substitution.

A “non-essential” amino acid residue is a residue that can be altered from the wild-type sequence of an embodiment polypeptide without abolishing or substantially altering one or more of its activities. Suitably, the alteration does not substantially alter one of these activities, for example, the activity is at least 20%, 40%, 60%, 70% or 80% of wild-type. An “essential” amino acid residue is a residue that, when altered from the herein disclosed sequences of FV TM or POI, or chimeric polypeptide, results in abolition of an activity of the parent molecule such that less than 20% of the parent activity is present.

In other embodiments, a variant polypeptide includes an amino acid sequence having at least about 50%, 55%, 60%, 65%, 70%, 75%, 80%, 85%, 90%, 91%, 92%, 93%, 94% 95%, 96%, 97%, 98% or more similarity to a corresponding sequence of a chimeric polypeptide as, for example, set forth in any one of SEQ ID NOs: 3, 6, 8, 10, 12, 14, 20, 22, 26 or 28

The present invention is further described by the following non-limiting Examples.

### EXAMPLES

### Example 1

### Foamy Virus gp47: Influenza H5NI Haemagglutinin HA1 Subunit Chimeric Polypeptides

The haemagglutinin HA1 subunit is the major neutralising antibody target. The haemagglutinin protein forms trimers with the TM subunit, HA2, being the trimer forming domain. This domain has been substituted with the Foamy Virus TM subunit (gp47) allowing trimerisation of the fusion protein and, further, assembly into virus-like particles (VLPs). In some embodiments, VLPs present the envelope proteins as 3D structures within a lipid membrane and thus resemble the native virion. Their particulate nature and size allow for efficient uptake by antigen presenting cells of the immune system for stimulation of the innate and adaptive immune response.

Various regions of the envelope polyprotein play a role in or affect the regulation of virion and subviral particle secretion. The leader peptide (LP) is essential for association with viral capsid and virion production and also regulates SVP secretion.

Mutations in the five ubiquitination sites of the LP and N terminal truncation up to amino acid 25 increases SVP release, however, the major determinant of SVP retention is the cytoplasmic domain of gp47. Deletion of the cytoplasmic domain (13 amino acids aa 409-421 of gp47) results in a 7-fold increase in SVP release (Stanke et al., J. Virol. 79(24): 15074-15083. 2005).

Substitution of the triple lysines of the ER retrieval signal (ERRS) at the C-terminus with serines (K417S, K418S, K419S) or deletion of the cytoplasmic domain does not adversely affect SVP formation but increases secretion. (Stange et al., J. Virol. 82(20): 9858-69, 2008; Stanke et al., 2005 (supra) and Shaw et al., 2003 (supra)).

Removal or mutation of the ERRS diverts virus budding from the ER membrane to the plasma membrane (Goepfert et al., J. Virol. 73: 7210-7217, 1999) and this switch in the sorting pathway is seen naturally in Equine Foamy Virus which does not have an ERRS and buds exclusively from the plasma membrane (Tobaly-Tapiero et al., J. Virol. 74: 4064-4073, 2000).

Mutation of the furin cleavage site (RKRK to RKRT; R572T) at the junction of SU and TM does not hinder SVP budding (Bansal et al., J. Virol. 74(6): 2949-2954, 2000).

In addition, several versions of HA1-gp47 fusion have been constructed to assess: i) the role of the FV leader peptide in chimeric VLP formation and secretion; ii) whether deletion of the cytoplasmic tail of gp47 allows assembly and facilitates secretion of chimeric VLPs and iii) the level of VLP production with a fusion sequence codon optimised for mammalian cell expression. The following fusion proteins were generated.

HA1-gp47 is a direct fusion of the H5N1 HA1 sequence to gp47 inclusive of the FV cleavage site (RKRK) which incorporated the mutation RKRT according to the study by Bansal et al., 2000 (supra) to prevent cleavage of the HA1 from gp47 by the cellular enzyme, furin. The lysine residues of the C-terminal ER retrieval signal (ERRS) of gp47 (K417; K418 and K418) were substituted with serine residues to enhance VLP secretion (Shaw et al., 2003 (supra)).

LPHA1-gp47 is a direct fusion of the FV LP to HA1-gp47 minus the N-terminal HA signal sequence (MEKIVLLFAIVSLVKS) and with mutations to the cleavage site RKRT and the ERRS.

HA-gp47ΔCD has the C-terminal 13 amino acids (the cytoplasmic tail of gp47) deleted.

HA-gp47 CO is a codon-optimised version of HA1-gp47. Schematic diagrams of the above constructs are shown in FIG. 3.

### Example 2

### Expression of HA1-gp47 Constructs in 293T Cells

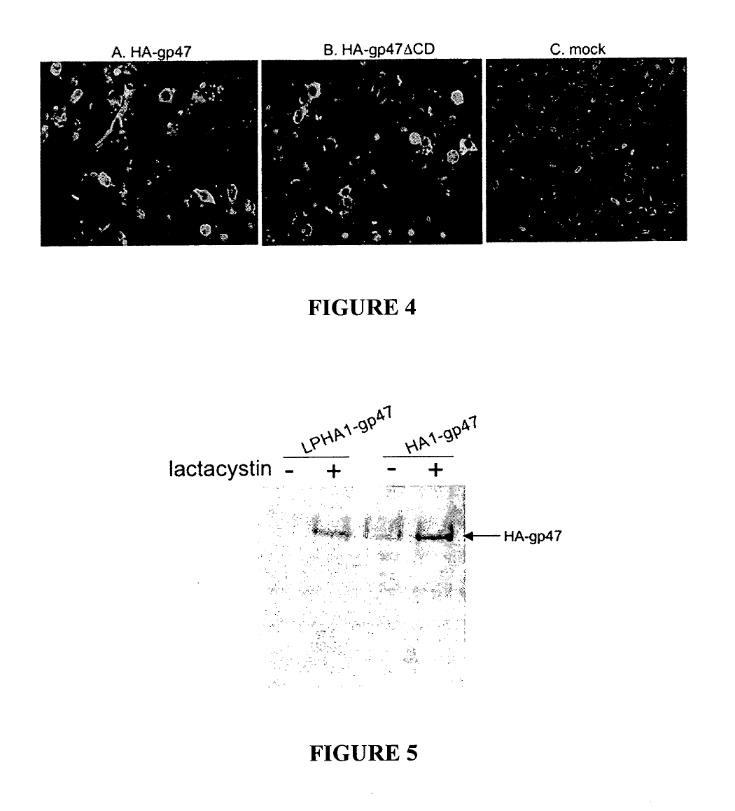

Expression of the HA1-gp47 and HA-gp47ΔCD fusion proteins was assessed by transfection of 232T cells with HA1-gp47 and HA-gp47ΔCD plasmid DNA and immunofluorescent staining of fixed cells grown on glass coverslips. Cells were fixed on day 2 post-transfection with cold methanol for 30 min at −20° C. and then blocked with 2% bovine serum albumin in PBS/0.25% TX100 for 1 hour at room temperature on a rocking platform. Cells were stained with monoclonal antibody to HA (MAb 149 WHO) diluted 1/500 in blocking solution for 1 hour at room temperature followed by 3 washes in PBS/0.25% TX100 and detection with a 1/2000 dilution of goat anti-mouse IgM ALEXA488 for 1 hour at room temperature. Cells were washed and nuclei stained with propidium iodine and images were collected using a Bio-Rad MRC 1024 confocal microscope mounted on a Nikon e600 upright epifluorescence microscope. FIG. 4 shows that both fusion constructs are expressed.

Expression of the LPHA1-gp47 construct was not detected by immunofluorescence labelling with MAb 149. Expression from this construct was tested in the presence or absence of the proteosome inhibitor, lactacystin (5 uM) and was detected with lactacystin but only by Western blot with a non-conformational anti-HA MAb 8D2 (FIG. 5) and not by immunofluorescent labelling with the conformational MAb 149. These results suggest that the addition of the LP causes instability and degradation of the protein. The LP does not appear to be tolerated in the context of these chimeric fusion proteins.

### Example 3

### Production of VLPs for HA1-gp47 Constructs

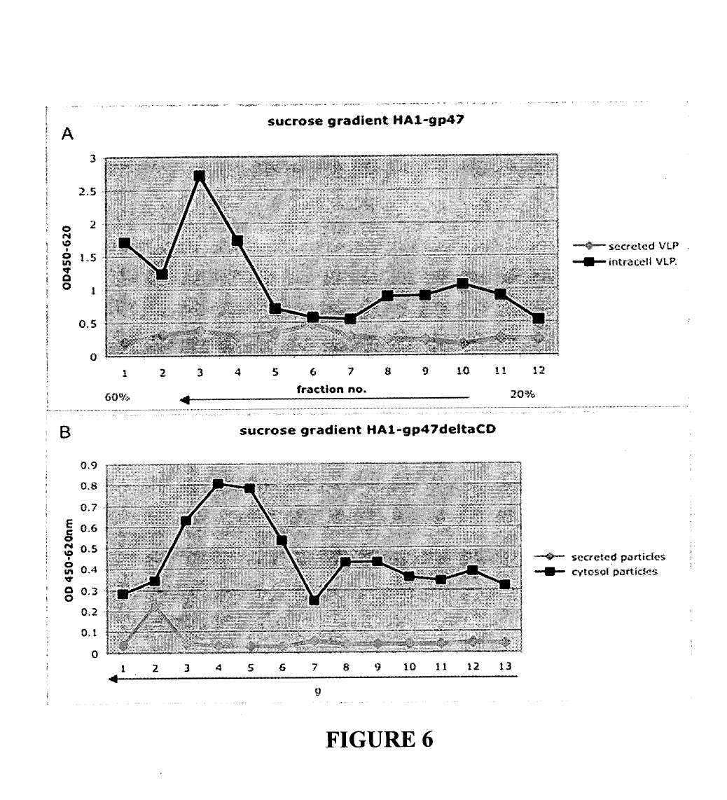

VLP formation for HA1-gp47 and HA1-gp47ΔCD was assessed by sucrose gradient sedimentation (FIG. 6). 293T cells were transfected with pHA1-gp47 or pHA1-gp47ΔCD and media and cells harvested day 2 post-transfection. Media was clarified of non-adherent cells by centrifugation at 1500 rpm for 5 minutes. Cell monolayers were washed with PBS, scraped and resuspended in PBS/1 mM EDTA (PBSE) and fractionated into a cytosolic fraction (containing intracellular particles) by three freeze-thawing cycles with vortexing, followed by centrifugation at 18000 g for 1 min. A soluble membrane protein preparation was obtained by vortexing the resulting pellet in the presence of PBSE with 1% NP-40 followed by centrifugation at 18000 g for 1 min. The cytosol fraction was vortexed well with a final concentration of 0.01% TX100. Media was concentrated to 3 mls in a Vivaspin 6 centrifugal device (MW cut off 100,000) for approximately 30 min at 3,000 rpm. The concentrated media and the cytosol fraction were applied to a sucrose step gradient composed of 1 ml each 70%, 60% 50% and 40% and 2 ml each of 30% and 20% sucrose (w/v) in PBS. Gradients were centrifuged for 5 hours at 38,000 rpm at 18° C. in an SW41 rotor. 500 ul fractions were collected from the bottom with a Beckman fraction recovery apparatus. 50 ul of each fraction was applied to Nunc Maxisorp plates for analysis by ELISA. Fractions were diluted 1/2 with carbonate coating buffer (pH9.6) and incubated overnight at 4° C. Contents were then discarded and wells blocked with 200 ul 3% skim milk in PBS for 1 hour at room temperature. Contents were discarded and wells incubated for 1 hour at RT with 100 ul of a 1/500 dilution of MAb 149 in 1% skim milk/PBS. Contents were discarded and wells washed 6 times with PBS/0.03% Tween 20. Bound antibody was detected with 100 ul goat anti-mouse IgM-HRP 1/2000 dilution in 1% skim milk/PBS for 1 hour at RT. Wells were then washed as before and 100 ul TMB (SureBlue Peroxidase substrate; KPL) added for 10 min omitting light and the reaction stoped with 100 ul H₂SO₄ stop solution. Detection of VLPs was measured by absorbance at 450-620 nm in an ELISA plate reader. Intracellular VLPs were detected but VLPs were not secreted from the cell.

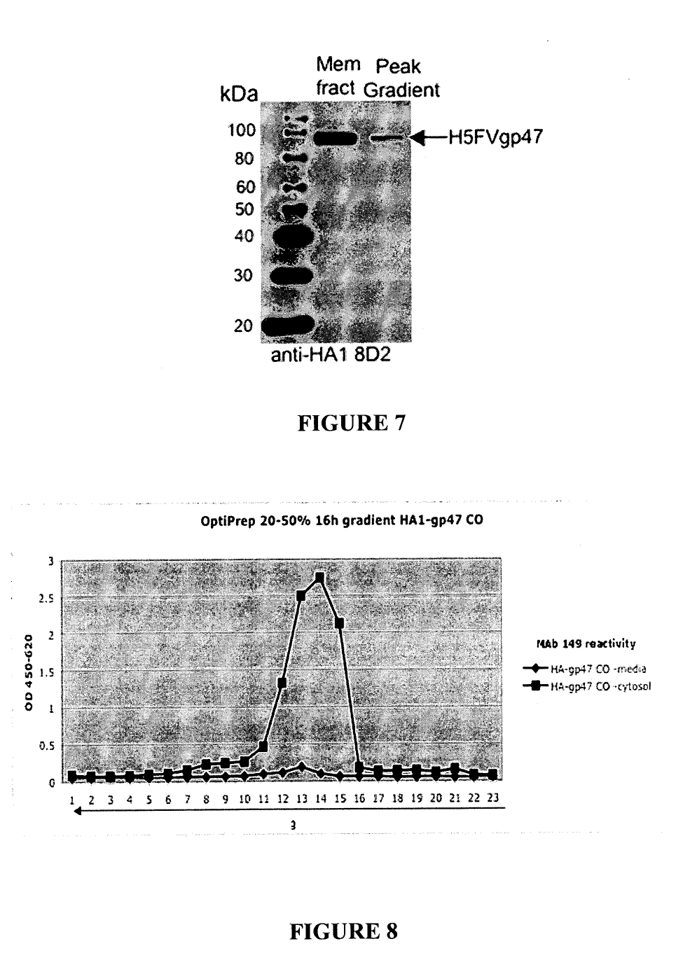

HA1-gp47 VLPs isolated by a sucrose step gradient were methanol precipitated and run on a 12% SDS-PAGE with the membrane fraction and the Western blot probed with anti-HA MAb 8D2 (Hytest) (FIG. 7). The fusion protein in both the membrane and VLP fraction was approximately the theoretical molecular weight of 85.27 kDa with carbohydrate modification of HA1 accounting for slight increase in size.

VLP formation for the HA1-gp47 Codon Optimised version was assessed by a two step process: sucrose cushion followed by an Iodixanol step gradient. Media from transfected cells was filtered through a 0.45 μm filter to remove non-adherant cells. Filtered media and the cytosol of transfected cells was centrifuges through 20% sucrose onto a 70% sucrose cushion (in NT: 10 mM Tris, 100 mM NaCl pH 7.4) at 38,000 rpm for 3 hours in a SW41 rotor. The interface between the 20 and 70% sucrose was then applied to a 20-50% Iodixanol gradient (OptiPrep; prepared in 0.85% NaCl, 10 mM Hepes pH7.4) and centrifuged for 16 hours at 38,000 rpm in an SW41 rotor. 500 ul fractions were collected from the top and 50 ul of each fraction was applied to Nunc Maxisorp plates for analysis by ELISA. Intracellular VLPs were detected but VLPs were not secreted from the cell (FIG. 8).

### Example 4

### Trimer Formation in HA1-gp47 Constructs

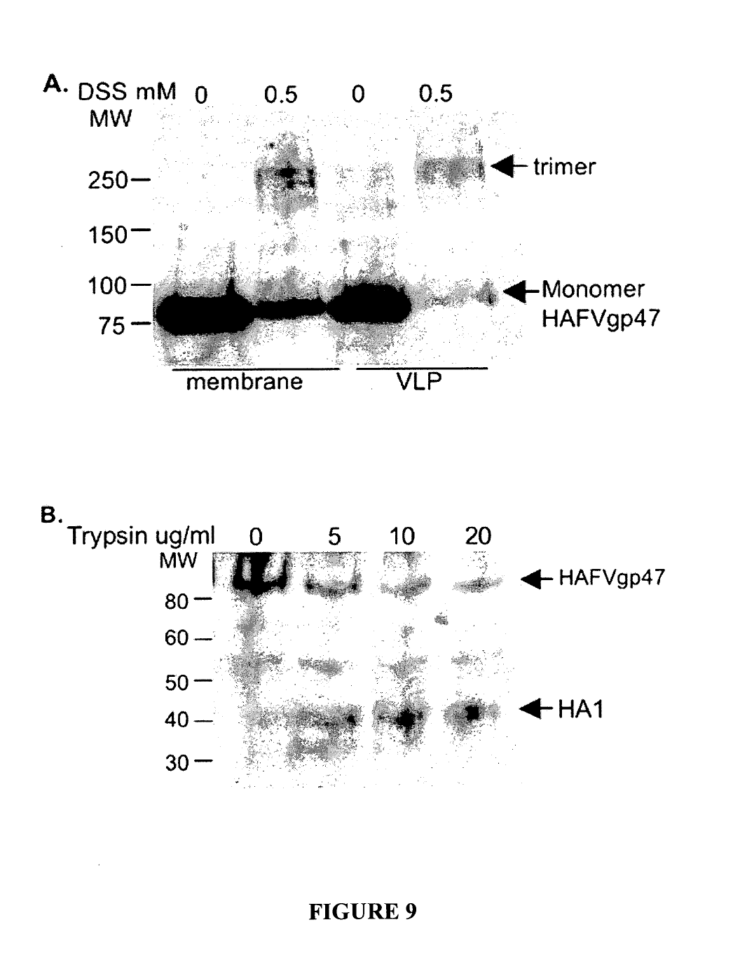

To assess trimer formation of HA1-gp47, the soluble membrane fraction and the VLP fraction was chemically cross-linked with 0.5 and 5 mM of the membrane permeable cross-linker, DSS (Pierce). Cross-linker was dissolved in DMSO and added to samples for 30 min at room temperature and later quenched with 1M Tris pH8.0 for 15 min at RT. The samples were run on 8% SDS-PAGE and the Western blot probed with anti-HA MAb 8D2 (FIG. 9A). Trimers at the expected size of approximately 255 kDa were detected in the membrane and VLP preparations. This result is consistent with trimer formation of influenza HA which occurs at the ER and is maintained in particles. For analysis of the conformation of HA by trypsin digestion, 40 ul of membrane fraction was incubated with 0, 5, 10 or 20 ug/ml trypsin on ice for 30 min. The reaction was stopped by the addition of 20 ug/ml aprotinin for 15 min. on ice and then boiled in Laemmli buffer and run on a 12% SDS-PAGE and Western blotted with MAb 8D2. Trypsin digestion of correctly folded HA molecules occurs at the multibasic cleavage site between HA1 and HA2. The furin cleavage site located between HA1 and gp47 consists of basic residues, RKRK, which have been mutated to RKRT to stop cleavage by furin. The remaining basic residues were cleaved by trypsin to release the 40 kDa HA1 subunit, indicative of a correct conformation of the globular HA1 bound or fused to the gp47 (FIG. 9B).

### Example 5

### HA1-gp47 VLPs are Immunogenic

To assess the immunogenicity of the HA-gp47 VLPs a small study was perform on 3 Balb/C mice. Mice were immunised with 100 ug of endotoxin free plasmid DNA followed by a VLP protein boost regimen (0.4 ng VLP HA equivalent) to assess if an antibody response occurred after DNA immunisation or after VLP boost.

### Immunisation Schedule

| | |
|---|---|
| Week 0 | 100 ug DNA intramuscular injection |
| Week 3 | 100 ug DNA intramuscular injection |
| Week 6 | 0.4 ng VLP boost FCA subcutaneous injection |
| Week 9 | 0.4 ng VLP boost FICA subcutaneous injection |

Mice were bled 2 weeks after the second dose of DNA and 2 and 3 weeks after the final VLP boost.

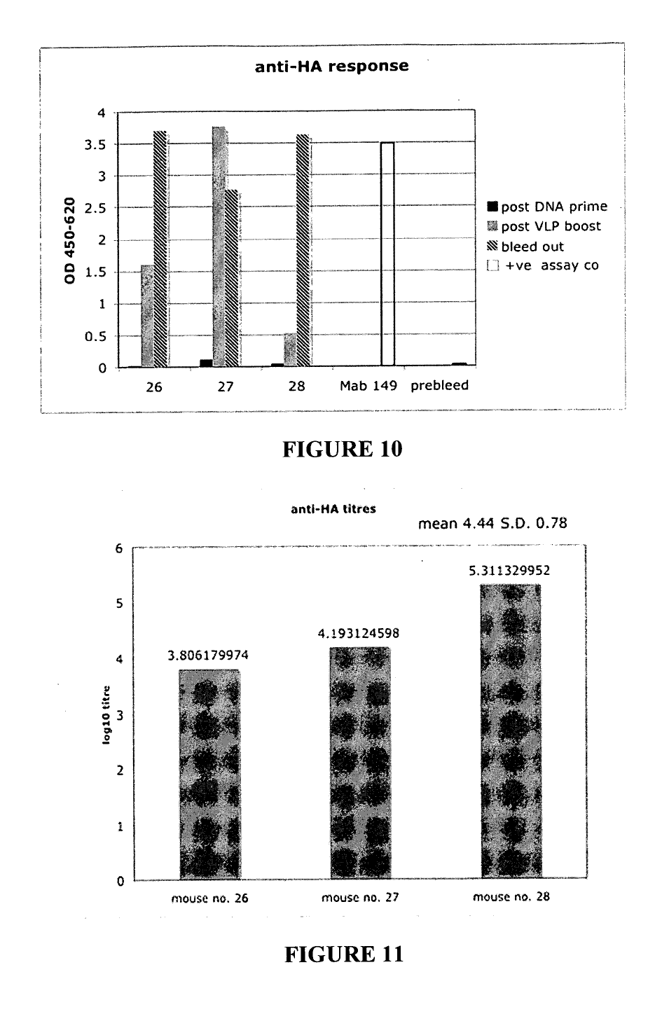

The anti-HA response in the mice sera was assessed by ELISA using 5 ug/ml of recombinant H5N1 HA (Protein Sciences). There was no antibody response after DNA immunisation but all mice responded after the VLP boosts (FIG. 10).

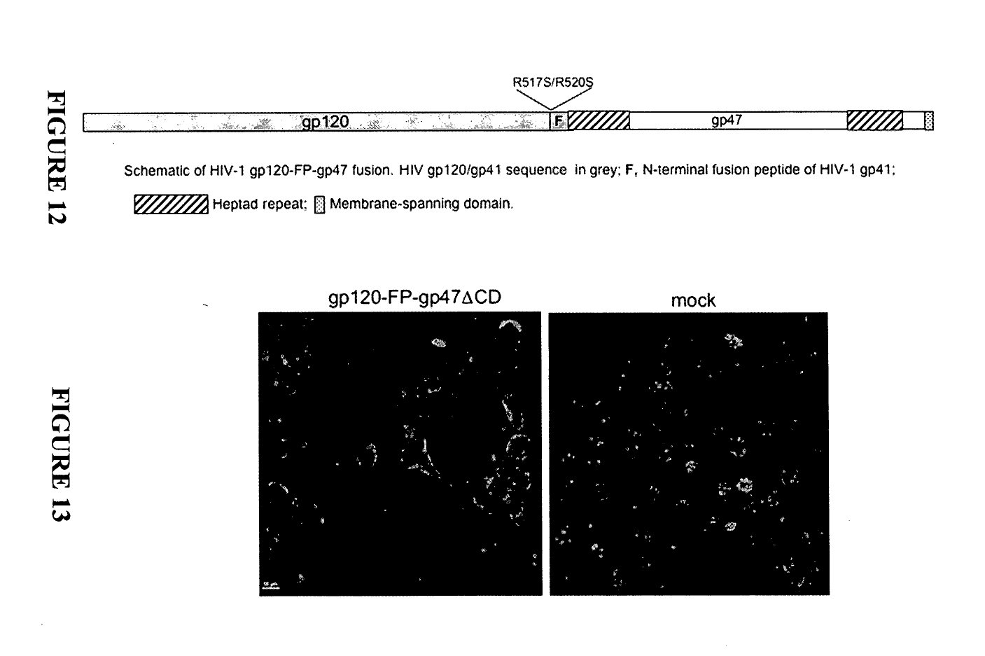

Endpoint anti-HA titres were calculated using the O.D. of 0.111 (the lowest dilution with a reading of the spare mouse sera) as the cut off (FIG. 11).

A haemagglutination inhibition assay was performed by the WHO Collaborating Centre for Reference and Research on Influenza (Parkville, Melbourne) on the final bleed mouse sera. Mouse no. 28 had a HI titre of 160 coinciding with the highest endpoint titre of the sera (log₁₀ 5.3). Mouse 26 and 27 were HI negative.

### Example 6

### HIV-1 gp120: FVgp47 Chimeric Constructs

A chimeric fusion protein was constructed consisting of the R2 sequence (Clade B, Accession No. AF128126) of gp120 and the fusion peptide domain of gp41 bound or fused directly to the N-terminal heptad repeat of FV gp47 minus the cytoplasmic tail (FIG. 12). The HIV-1 sequence encompasses the HIV-1 furin cleavage site between the gp120 and the HIV-1 TM protein, gp41. The furin cleavage site, REER, was mutated to SEES (R517S and R520S of the gp160 sequence) to halt cleavage of the fusion protein (Quinnan et al., J. Virol. 79(6): 3358-3369, 2005). The fusion sequence was codon optimised for expression in mammalian cells.

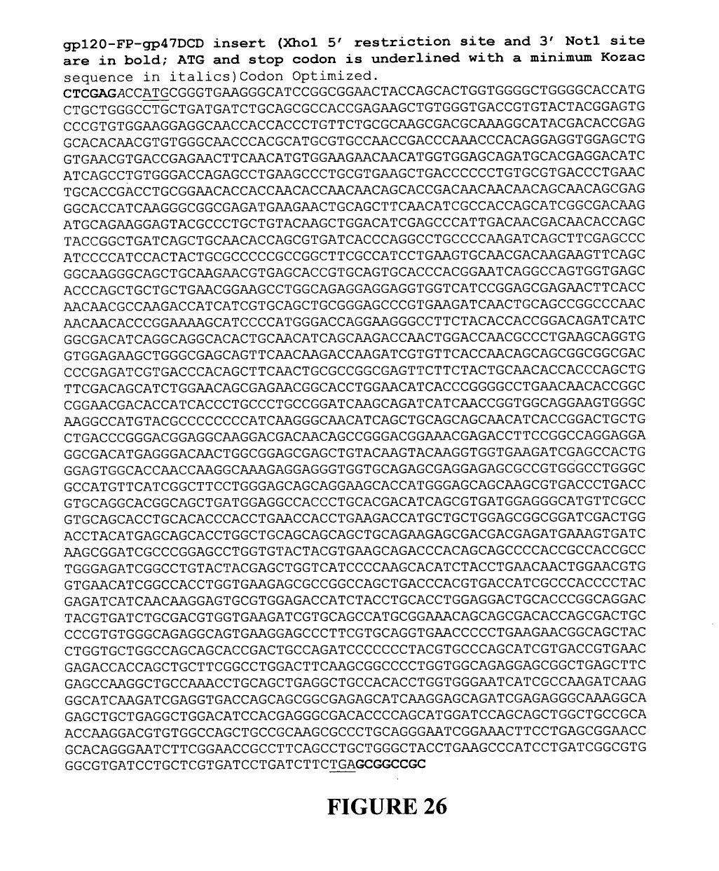

Genes were synthesised with a Kozac sequence and a XhoI restriction enzyme site at the 5′ end and a stop codon and NotI site at the 3′ end. The gene was subcloned from a pBlueScript vector into pClneo using the XhoI and NotI restriction sites. The insert used with restriction sites and stop codon is shown in FIG. 26.

Expression of the gp120-FP-gp47ΔCD fusion protein was assessed by transfection of 293T cells with pCI gp120-FP-gp47ΔCD plasmid DNA and immunofluorescent staining of fixed cells grown on glass coverslips. Cells were fixed on day 2 post-transfection with cold methanol for 30 min at −20° C. and then blocked with 2% bovine serum albumin in PBS/0.25% TX100 for 1 hour at room temperature on a rocking platform. Cells were stained with monoclonal antibody to gp120 (MAb 2G12) diluted 1/4000 in blocking solution for 1 hour at room temperature followed by 3 washes in PBS/0.25% TX100 and detection with a 1/2000 dilution of goat anti-mouse IgM ALEXA488 for 1 hour at room temperature. Cells were washed and nuclei stained with propidium iodine and images were collected using a Bio-Rad MRC 1024 confocal microscope mounted on a Nikon e600 upright epifluorescence microscope. FIG. 13 shows that the fusion construct is expressed and reacts with a neutralising MAb.

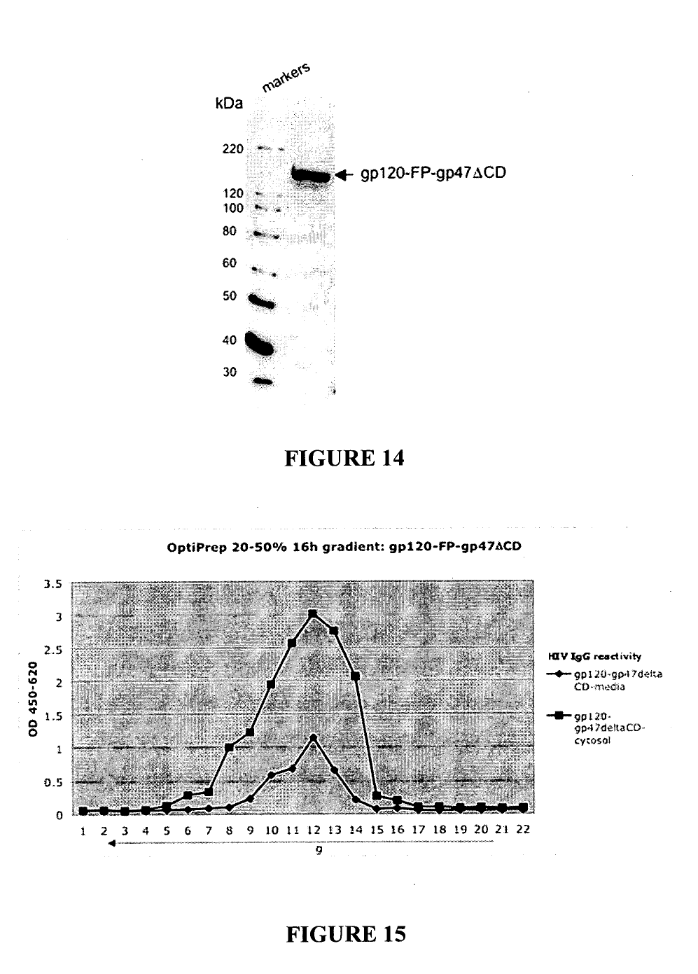

A soluble membrane fraction of gp120-FP-gp47ΔCD fusion protein was run on a 4-15% gradient SDS-PAGE and the Western blot probed with HIV-1 plasma IgG. FIG. 14 shows that the fusion protein runs at a molecular mass of approximately 140-180 kDa.

VLP formation was assessed by OptiPrep equilibrium gradient. 293T cells were transfected with pCIgp120-FP-gp47ΔCD and media and cells harvested day 2-post-transfection. Media from transfected cells was filtered through a 0.45 μm filter to remove non-adherant cells. Cell monolayers were washed with NT (100 mM Tris 150 mM NaCl, pH 7.4), scraped and resuspended in NT and fractionated into a cytosolic fraction (containing intracellular particles) by three freeze-thawing cycles with vortexing, followed by centrifugation at 18000 g for 1 min. A soluble membrane protein preparation was obtained by vortexing the resulting pellet in the presence of NT with 1% NP-40 followed by centrifugation at 18000 g for 1 min to remove the nuclei. Media and VLPs in the cytosol fraction were sedimented through 20% sucrose onto a 70% sucrose cushion for 3 hours at 38,000 rpm in an SW41 rotor. The interface between 20 and 70% sucrose was collected and diluted with 0.85% NaCl/10 mM Hepes-NaOH pH 7.4 and applied to a 20-50% OptiPrep density gradient. Gradients were centrifuged for 16 hours at 38,000 rpm at 18° C. in an SW41 rotor. 500 ul fractions were collected from the bottom with a Beckman fraction recovery apparatus. 50 ul of each fraction was applied to Nunc Maxisorp plates for analysis by ELISA. Gradient fractions were detected with HIV plasma IgG. Both intracellular and secreted VLPs were detected with HIV-1 plasma IgG (FIG. 15).

To determine binding of VLPs to conformational, neutralising monoclonal antibodies, gradient prepared VLPs was assessed by ELISA with MAb, b12 and 2G12. MAb b12 binds to an epitope overlapping the CD4 receptor-binding site in a conformation which resembles the CD4-bound structure. b12 is the most potently and broadly neutralising anti-gp120 antibody. Monoclonal antibody 2G12 recognises a discontinuous epitope of glycans on the silent face of gp120 and is broadly neutralising but unable to neutralise Clade C viruses (reviewed by Pantophlet and Burton, Annu. Rev. Immunol. 24: 739-769, 2006).

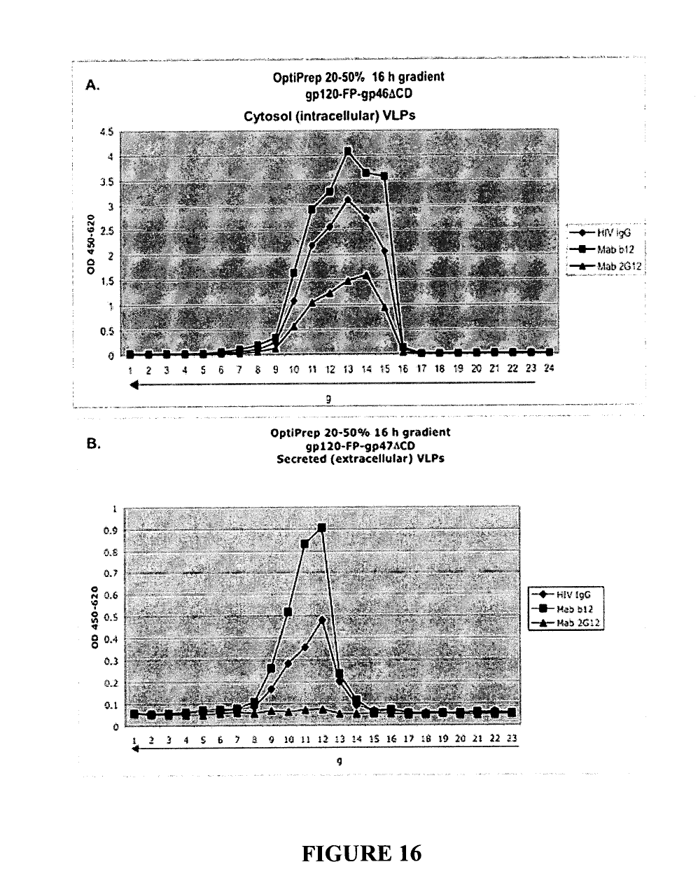

VLPs are secreted and both intracellular and secreted VLPs are reactive with monoclonal antibody b12, however the secreted VLPs are no longer recognised by the monoclonal, 2G12. This suggests that there may be differences in the glycosylation of gp120 between secreted and intracellular particles (FIG. 16).

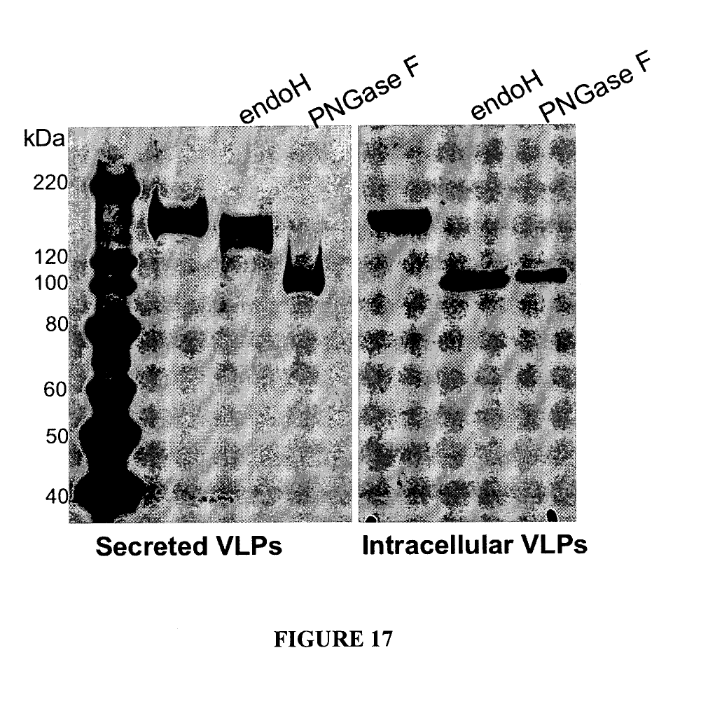

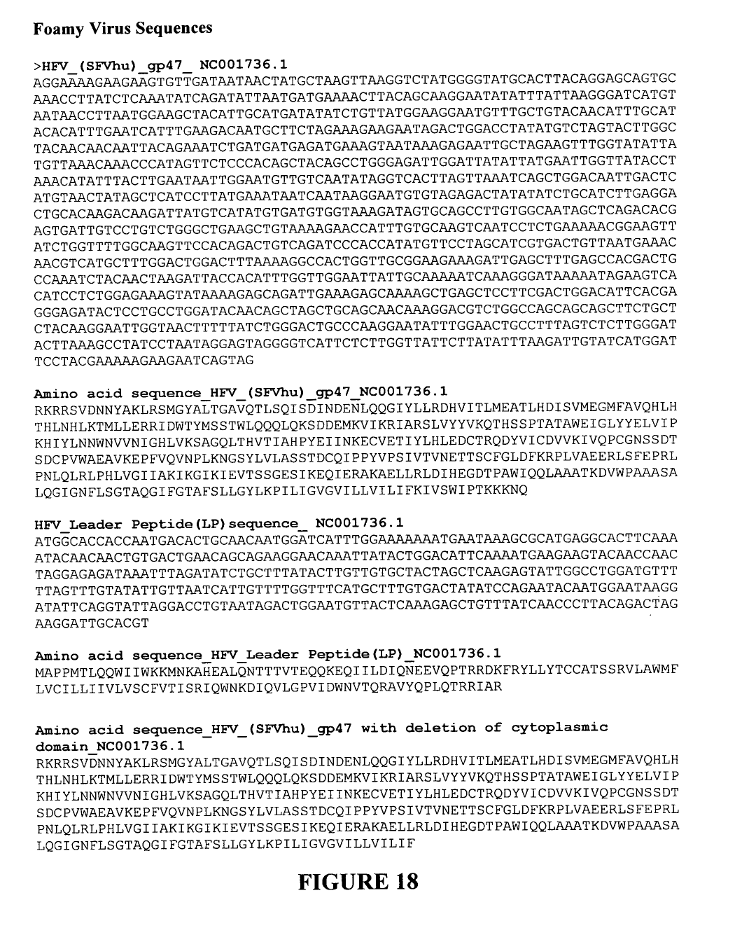

To assess the glycosylation pattern of secreted and intracellular VLPs VLPs were either left untreated or treated with the enzymes, endoglycosidase H (endo H) or PNGase F and then visualised by SDS-PAGE and Western blotting with HIV plasma IgG. The envelope protein of secreted VLPs is nearly all protected from endo H digestion, indicating that the carbohydrates on these VLPs are further modified in the Golgi to hybrid/complex glycans. The envelope protein of intracellular VLPs, in contrast, is completely digested with endo H, indicating the carbohydrates are high mannose in nature. PNGase F treatment causes digestion of both high mannose and complex carbohydrates as show in FIG. 17.

### Example 7

### Analysis of Trimer Formation of gp120-FP-gp47δCD on Vlps by Chemical Cross-Linking

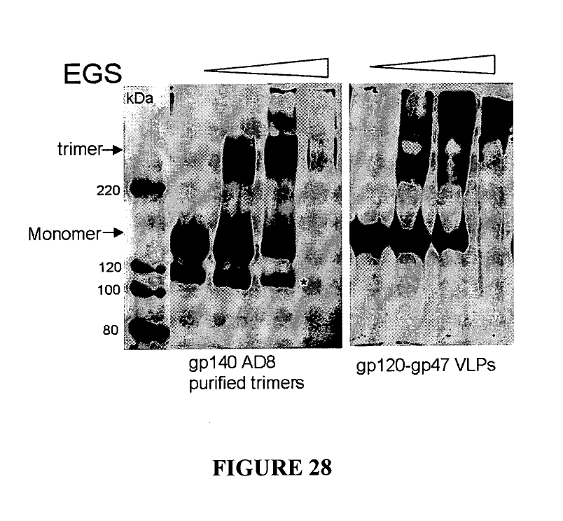

To assess trimer formation of gp120-FP-gp47ΔCD on the VLP, intracellular VLPs were isolated by sedimentation through 20% sucrose in PBS and the pellet concentrated 10-fold in a Vivaspin 20 centrifugal device with a MW cut off of 300,000. 20 μl aliquots were either left untreated or cross-linked with 0.04, 0.2 or 1 mM EGS (Ethylene glycolbis(succinimidylsuccinate). In parallel soluble HIV-1 trimer gp140 (AD8) purified by size exclusion chromatography was similarly cross-linked as an indicator of monomer, dimer and trimer species. Cross-linker was dissolved in DMSO and added to samples for 30 min at room temperature and later quenched with 1M Tris pH8.0 for 15 min at RT. The samples were run on 6% SDS-PAGE and the Western blot probed with HIV IgG. Trimers at the expected size of approximately 420 kDa were detected as well as some higher order oligomers (FIG. 28).

### Example 8

### Confirmation of VLP Formation by Electron Microscopy

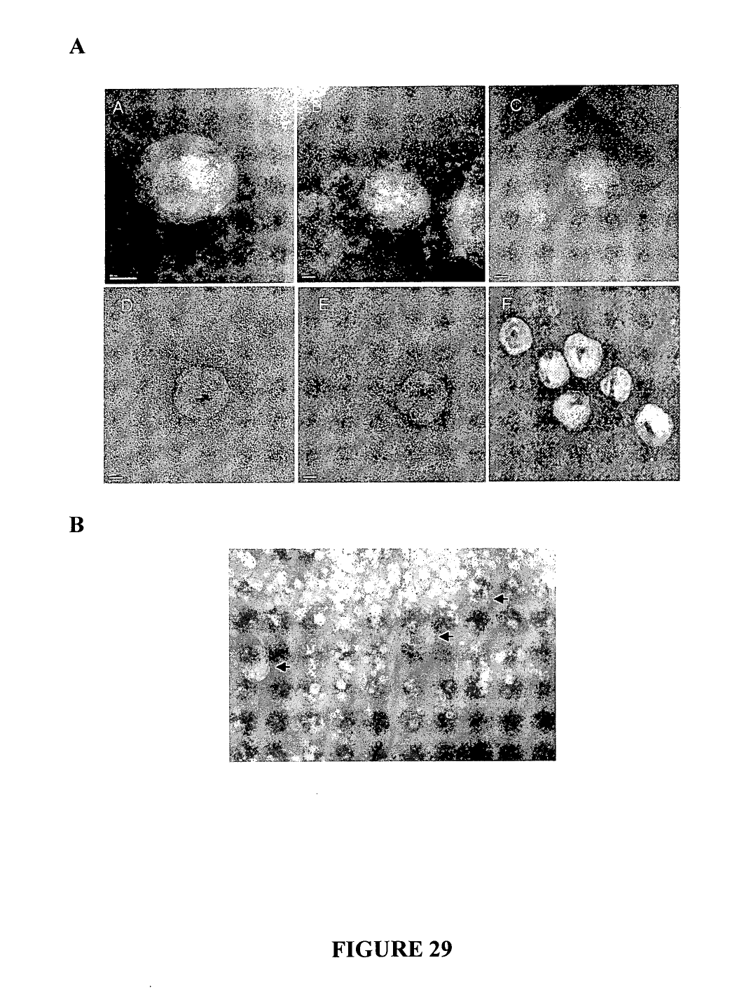

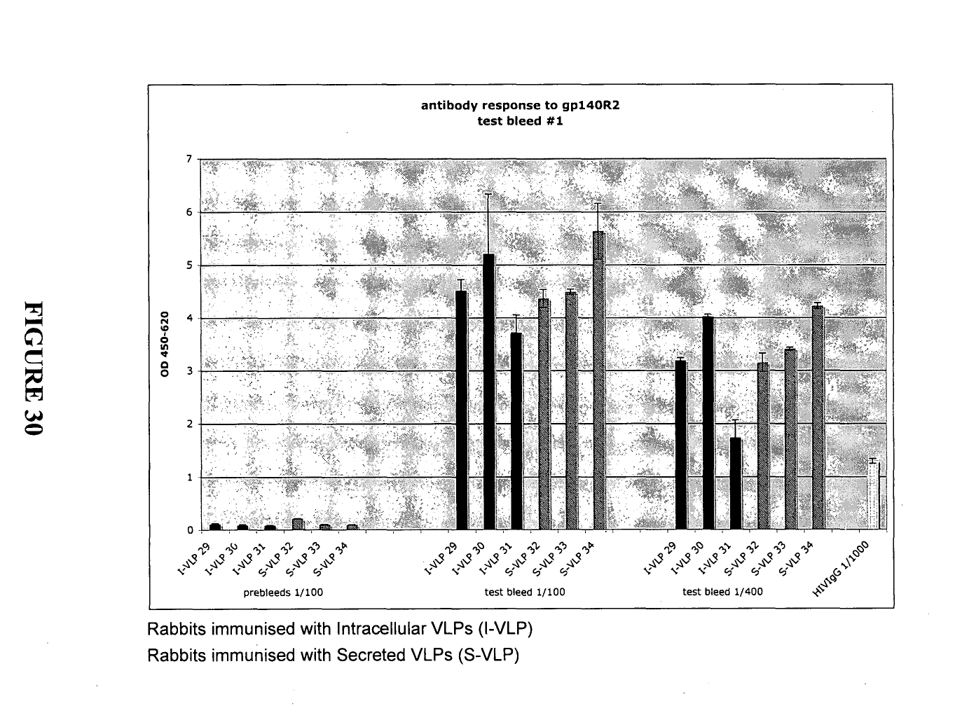

Intracellular and secreted VLPs were prepared from two 75 cm² flasks of transfected 293T cells. Media were harvested day 2 post transfection and filtered through a 0.45μ filter. VLPs were pelleted from the media and cytosol fraction of 293T cells through 20% sucrose onto a 70% sucrose cushion in 10 mM HEPES/0.85% NaCl pH 7.4 for 3 hours at 38,000 rpm in an SW41 rotor. The interface between 20 and 70% sucrose was collected and diluted 1:2 with 0.85% NaCl/10 mM Hepes-NaOH pH7.4 and applied to a 20-50% OptiPrep density gradient. Gradients were centrifuged for 16 hours at 38,000 rpm at 18° C. in a SW41 rotor. 500 ul fractions were collected from the top and 50 μl of each fraction coated on a Nunc Maxisorp plate for analysis by ELISA. Gradient fractions were detected with HIVplasma IgG followed by Goat anti-human IgG-HRP and then TMB substrate (KPL). Peak fractions of intracellular and secreted VLPs were pooled and concentrated in a Vivaspin 20 centrifugal device (300,000 MW cut off) and stored at 4° C. until negatively stained with uranyl acetate and analysed by electron microscopy (FIG. 29). All particles were between 50-70 nm in diameter. Intracellular particles resembled the subviral particles of hepadnaviruses as indicated in panel F of FIG. 29A, while secreted VLPs (panel A-C FIG. 29A) had a more fuller, rounded appearance.

### Example 9

### Immune Response to Secreted and Intracellular VLPs in Rabbits

To assess the immunogencicity of the gp120-FP-gp47ΔCD VLPs two groups of three rabbits were immunised four 5 μg doses of either secreted or intracellular VLPs. Rabbits were immunised with Alum adjuvant subcutaneously. VLP doses were quantified by Western blot and the Li-Cor Odyssey Infrared Imaging System against known amounts of recombinant gp140(AD8) and gp140(R2) (the envelope polyprotein absent the membrane spanning domain and the cytoplasmic tail).

| | |
|---|---|
| Week 0 | 1^{st} dose 5 μg VLP |
| Week 3 | 2^{nd} dose 5 μg VLP |
| Week 6 | 3^{rd} dose 5 μg VLP |
| Week 9 | 4^{th} dose 5 μg VLP |

Rabbits were bleed 10 days after the 2^{nd} dose (test bleed#1) and 10 days after the 3^{rd} dose (test bleed#2) and bled out 10 days after the final dose.

The anti-gp120 response was assessed by ELISA using gp140(R2), a soluble trimer of HIV-1 envelope of the R2 sequence, the sequence used for construction of the VLPs. 50 μl of 5 μg/ml gp140R2 was coated onto Sarstaedt 96-well immunoplates overnight at 4° C. Wells were blocked with 200 μl 5% skim milk in PBS for 1 hr at RT. 50 μl of I/100 dilutions of each prebleed rabbit sera or 1/100 and 1/400 dilutions of test bleed#1 rabbit sera was applied to the plates in 1% skim milk/PBS/0.05% tween 20 as diluent for 1 hour at RT. HIVIgG at 1/1000 dilution was included as a positive control. Plates were washed 6 times with PBS/0.05% Tween 20 and then incubated for 1 hour with goat anti-rabbit IgG-HRP at 1/2000 dilution in diluent. Plates were washed with 6 times with PBS/0.05% Tween 20 and 50 μl TMB (KPL) substrate added and covered from light for 10 min. 50 μl stop solution (0.5M H₂SO₄) was added and absorbance was read at 450-620 nm with a ELISA plate reader (FIG. 30). The reactivity of the rabbit test bleed sera to gp140R2 indicates that all the rabbits have seroconverted after two doses of VLPs.

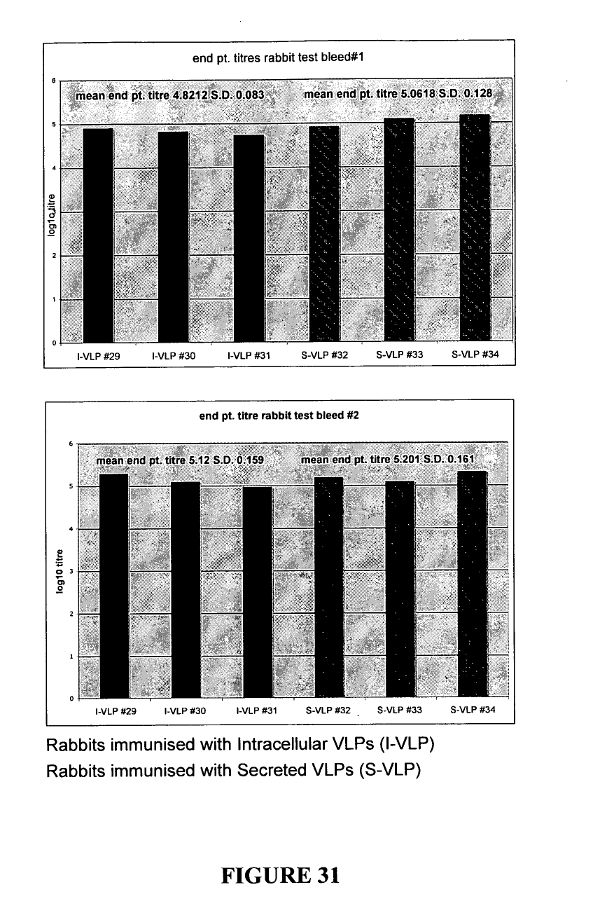

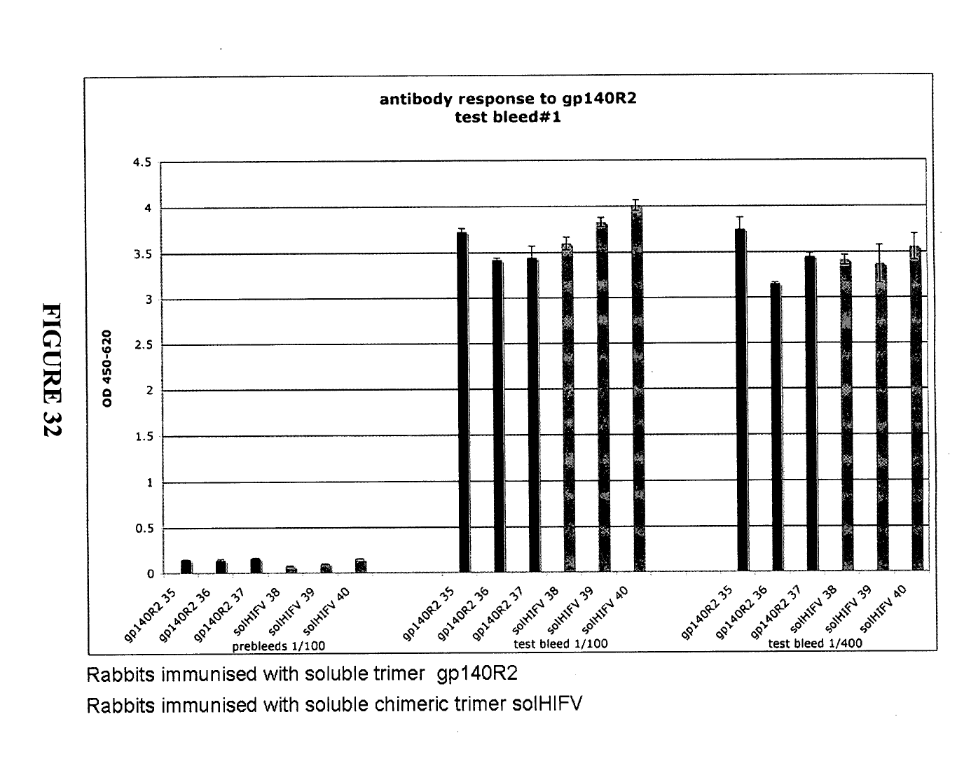

Endpoint titrations were performed on plates coated with 0.5 μg/ml gp140R2. 2-fold dilutions of pooled prebleeds for I-VLP and S-VLP rabbit groups starting from 1/6.25 and for individual test bleeds of rabbit sera starting at 1/200 or 1/400 were prepared and 500 incubated on coated plates as described above. Anti-gp140 endpoint titres for test bleed #1 were calculated using the O.D. of 0.096 and 0.102 (the lowest dilution with a reading of the pooled bleeds for I-VLP and S-VLP rabbits, respectively) as the cut off and intercept end point titre values calculated from a curve of best fit for individual rabbit sera. Anti-gp140 endpoint titres for test bleed #2 were calculated using the O.D. of 0.127 and 0.098 (the lowest dilution with a reading of the pooled bleeds for I-VLP and S-VLP rabbits, respectively) as the cut off. (FIG. 31). The same or similar methods are used to assess the antibody response to soluble trimeric chimeric proteins.

### Example 10

### Immune Response Study Of Soluble Trimers, gp140R2 and solHIFV in Rabbits (see Examples 11, 12, 13, 14, 15 and 16 for Details)

To assess the immunogencicity of the soluble trimer version of gp120-FP-gp47 (solHIFV) two groups of three rabbits were immunised with four 5 μg doses of either solHIFV or the positive control gp140R2. Rabbits were immunised with Alum adjuvant subcutaneously. Trimers were purified by lentil lectin-sepharose affinity chromatography from cell culture supernatant and doses were quantified by Western blot and the Li-Cor Odyssey Infrared Imaging System against known amounts of recombinant gp140(AD8) and gp140(R2).

| | |
|---|---|
| Week 0 | 1^{st} dose 5 μg trimer |
| Week 3 | 2^{nd} dose 5 μg trimer |
| Week 6 | 3^{rd} dose 5 μg trimer |
| Week 9 | 4^{th} dose 5 μg trimer |

Rabbits were bleed 10 day after the 2^{nd} dose (test bleed#1) and assessed for antibody responses to gp140R2 by ELISA.

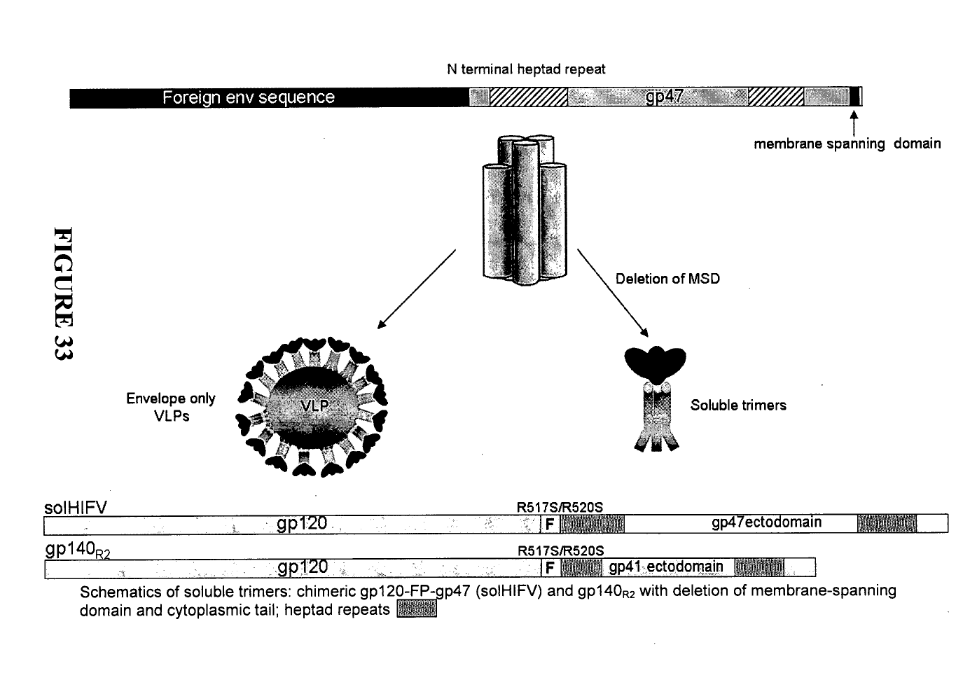

50 μl of 0.5 μg/ml gp140R2 was coated onto Sarstaedt 96-well immunoplates overnight at 4° C. Wells were blocked with 200 μl 5% skim milk in PBS for 1 hr at RT. 50 μl of I/100 dilutions of each prebleed rabbit sera or 1/100 and 1/400 dilutions of test bleed#1 rabbit sera was applied to the plates in 1% skim milk/PBS/0.05% tween 20 as diluent for 1 hour at RT. Plates were washed 6 times with PBS/0.05% Tween 20 and then incubated for 1 hour with goat anti-rabbit IgG-HRP at 1/2000 dilution in diluent. Plates were washed with 6 times with PBS/0.05% Tween 20 and 50 μl TMB (KPL) substrate added and covered from light for 10 min. 500 stop solution (0.5M H₂SO₄) was added and absorbance was read at 450-620 nm with an ELISA plate reader (FIG. 32). The reactivity of the rabbit test bleed sera to gp140R2 indicates that all the rabbits have seroconverted after two doses of soluble trimer.

### Example 11

### Soluble Trimer Version of gp120-FP-gp47

A chimeric fusion protein was constructed consisting of the R2 sequence (Clade B, Accession No. AF128126) of gp120 and the fusion peptide domain of gp41 fused directly to the N-terminal heptad repeat of FV gp47 minus the membrane spanning domain and the cytoplasmic tail. The HIV-1 sequence encompasses the HIV-1 furin cleavage site between the gp120 and the HIV-1 TM protein, gp41. The furin cleavage site, REER, was mutated to SEES (R517S and R520S of the gp160 sequence) to halt cleavage of the fusion protein (Quinnan et al., 2005 (supra)). The fusion sequence was codon optimised for expression in mammalian cells. The construct (HIFV) was designed for expression of soluble chimeric trimers which are released to media of transfected cells. A codon optimised soluble timer of gp140R2 was also constructed for comparative analysis with the performance of the HIFV trimer (FIG. 33 provides a schematic representation of soluble trimer constructs).

### Example 12

### Cloning Strategy for Soluble Chimeric Trimer Construct (HIFV)

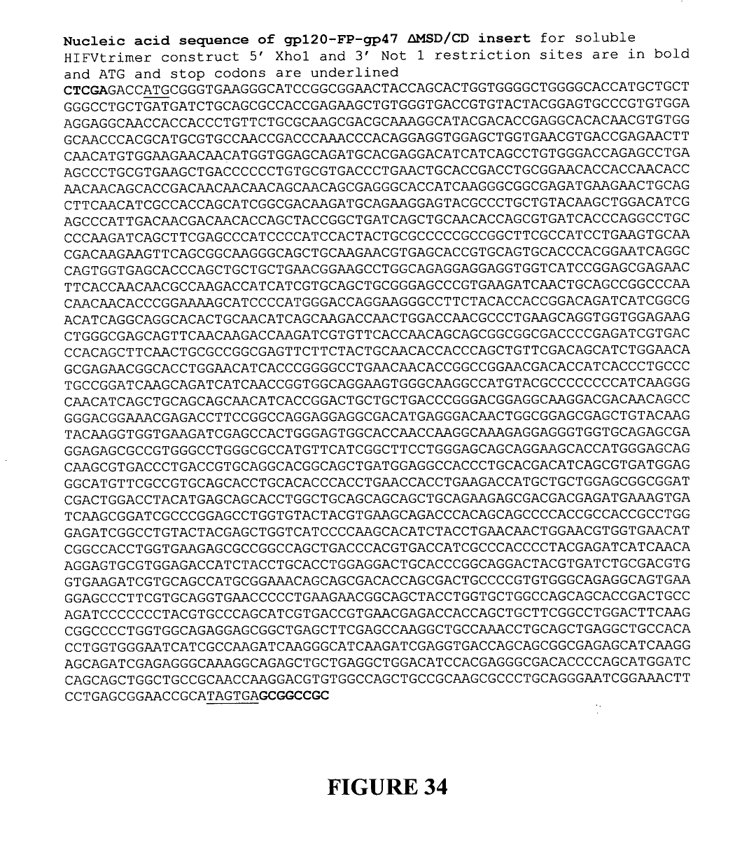

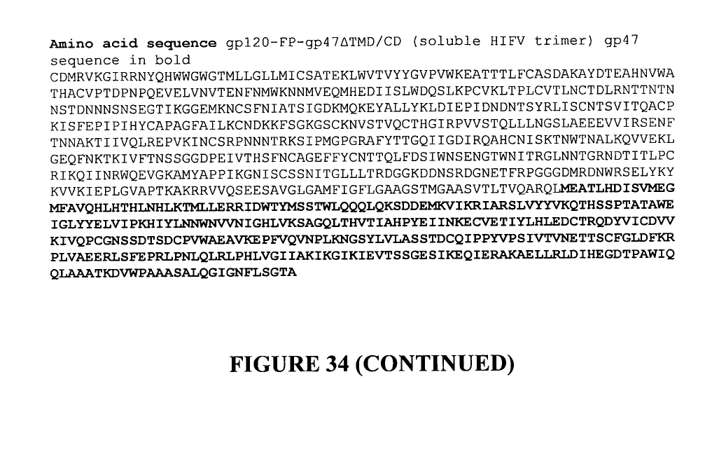

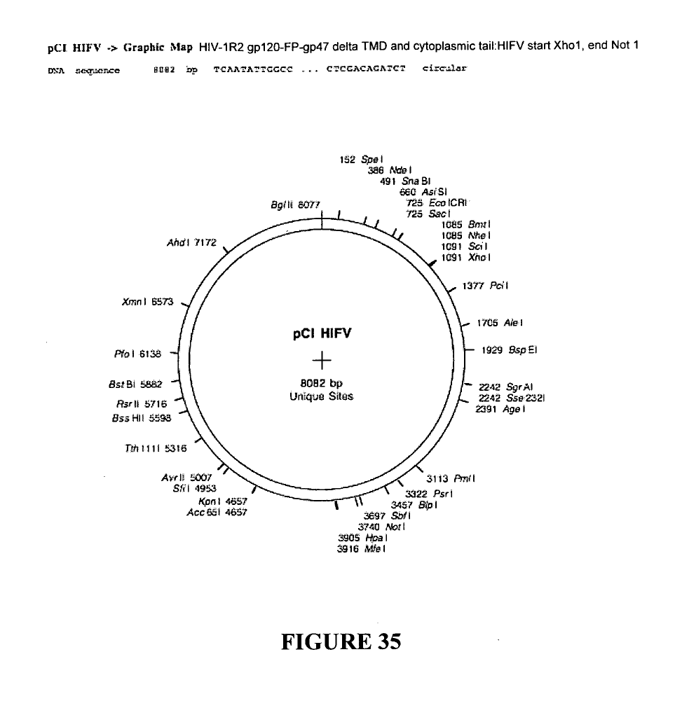

A codon optimised gp120-FP-gp47 fusion which terminated with two sequential stop codons at the start of the membrane spanning domain (MSD) of gp47 was generated by insertion of a synthetic gene sequence starting from a Blpl restriction site at nucleotide 3457 of the pCI gp120-FP-gp47ΔCD sequence and ending in two stop codons and a Nott restriction site. The synthetic gene sequence was obtained from Genescript for cloning into the mammalian expression vector pCIneo (Promega). The gene was subcloned from a pUC57 vector into pClneo using the Blpl and NotI restriction sites. The gp120-FP-gp47ΔMSD nucleic acid sequence (SEQ ID NO: 25), amino acid sequence (SEQ ID NO: 26) and plasmid map (FIGS. 34 and 35) are provided.

### Example 13

### Analysis of Trimer Formation of Soluble HIFV by Chemical Cross-Linking

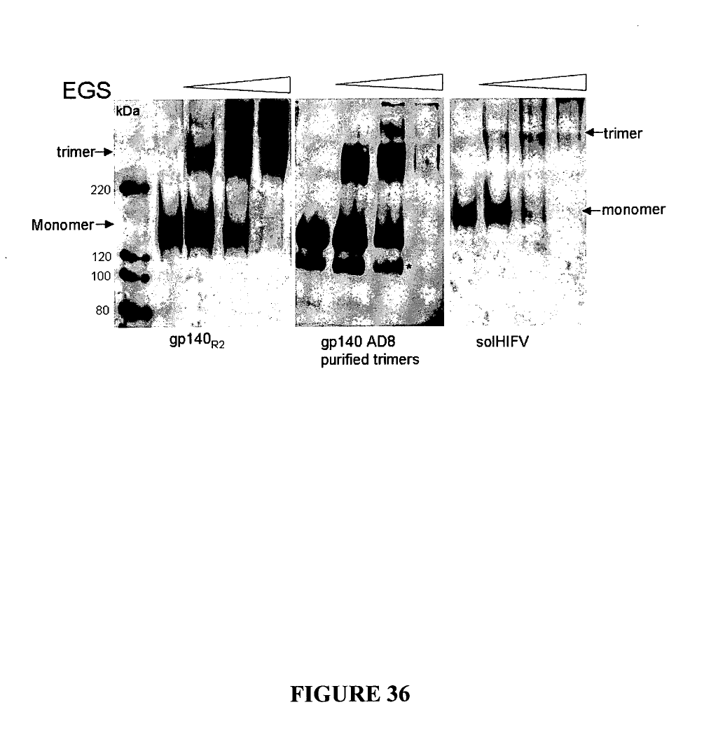

To assess trimer formation, media from cells transfected with pCI HIFV and pCI gp140R2 was filtered through 0.45μ filter and trimers isolated by sedimentation through 20% sucrose in PBS and the pellet concentrated 10-fold in a Vivaspin 20 centrifugal device with a MW cut off of 100,000. 20 μl aliquots were either left untreated or cross-linked with 0.04, 0.2 or 1 mM EGS (Ethylene glycolbis(succinimidylsuccinate). In parallel soluble HIV-1 trimer gp140 (AD8) purified by size exclusion chromatography was similarly cross-linked as an indicator of monomer, dimer and trimer species. Cross-linker was dissolved in DMSO and added to samples for 30 min at room temperature and later quenched with 1M Tris pH8.0 for 15 min at RT. The samples were run on 6% SDS-PAGE and the Western blot probed with HIV IgG. Trimers at the expected size of approximately 420 kDa were detected as well as some higher order oligomers (FIG. 36).

### Example 14

### Binding of Soluble Trimers to Neutralising Monoclonal Antibodies

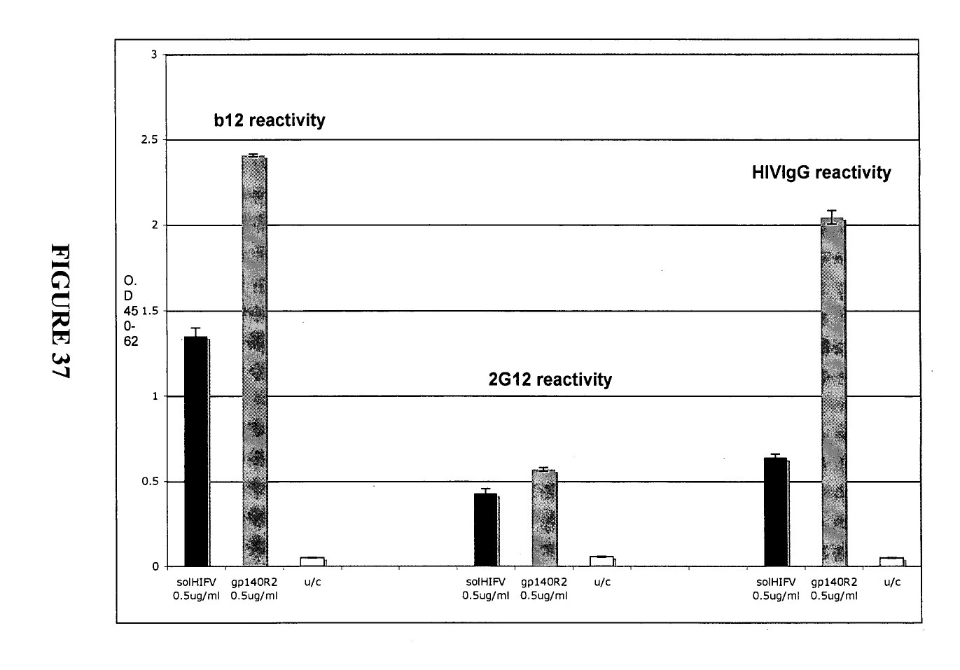

Soluble trimers were purified from media by lentil lectin-sepharose and quantified against known concentrations of gp140 (AD8) by Western blotting with anti-gp120 monoclonal antibody and the Li-Cor Odyssey Infrared Imaging System. Equivalent concentrations of gp140R2 and soluble HIFV (0.5 μg/ml) were coated on a Nunc Maxisorp plate and binding of human monoclonal antibodies, b12 and 2G12 and HIV plasma IgG to trimers assessed by ELISA. u/c, indicates uncoated wells (FIG. 37). Both trimers reacted to 2G12 similarly. Differences in reactivity to HIVIgG may be due to gp140R2 containing additional HIV-1 epitopes in the gp41 region of the gp140 sequence, which HIFV has substituted with FV gp47. Differences in reactivity of b12 may be accounted for differences in the heterogeneity, with gp140 having monomeric and dimeric forms reactive to b12 in addition to trimer as described below.

### Example 15

### Assessment of Monomer, Dimer and Trimer Species by Blue Native-PAGE

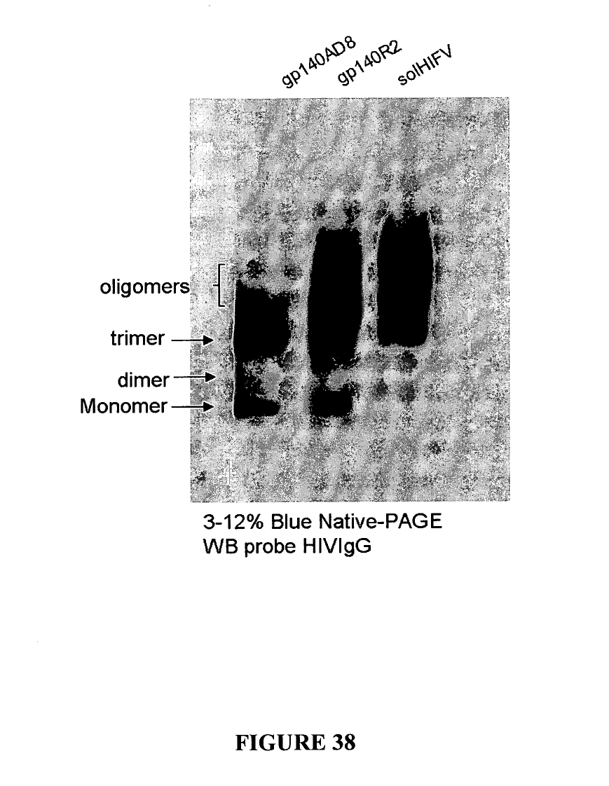

To assess the degree heterogeneity of the soluble trimer HIFV (solHIFV), 200 ng of lentil lectin-sepharose purified solIFV and gp140R2 and size exclusion purified gp140 (AD8) were run on a 3-12% Blue Native PAGE precast gradient gel (Invitrogen) according to instructions for the Novex BisTris Native PAGE system (Invitrogen). Separated proteins were transferred to PVDF membrane using the semi dry Transblot system (Bio-Rad) and the membrane probed using HIVIgG (1/5000 dilition), anti-hu IgG-HRP followed by chemiluminesence detection using ECL reagent (GE Healthcare). Native PAGE analysis (see FIG. 38) indicates that the soluble HIFV is composed of trimer and higher order oligomers (including dimers of the trimer) with little or no monomer or dimer species evident. In contrast, gp140R2 and gp140(AD8) both contain a substantial proportion of monomer as well as some dimer.

### Example 16

### Stability Test of Soluble HIFV Trimers

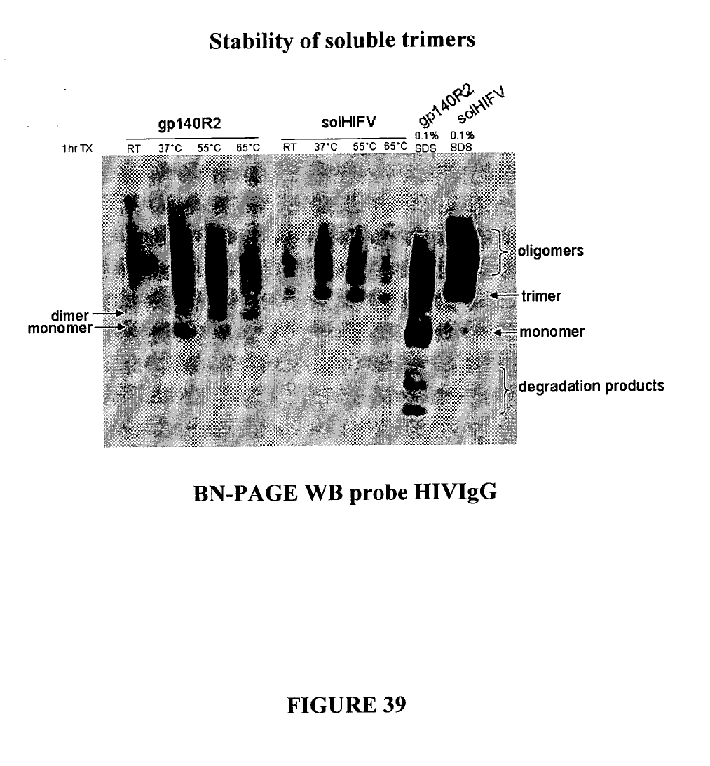

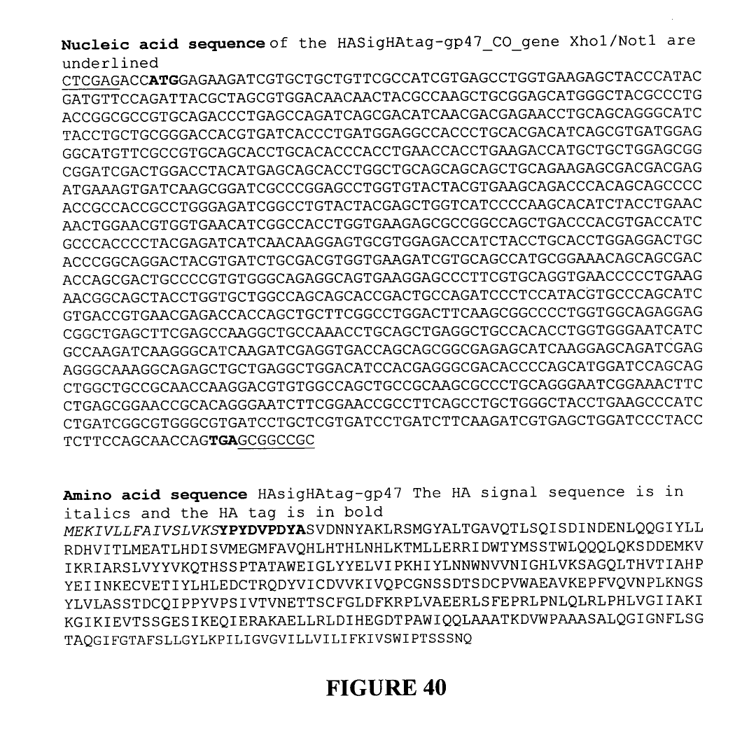

To assess the relative stability of the soluble HIFV trimers compared with gp140R2 the trimers were incubated for 1 hour at various temperatures (room temperature, 37° C., 55° C. and 65° C.) or in the presence of the anionic detergent, SDS (0.1%), which causes dissociation of gp140 into monomers (Sanders et al., 2002 J. Virology 76:8875). After treatment the samples were placed on ice and then loaded onto a 3-12% Blue Native PAGE. Separated proteins were transferred to PVDF membrane and the membrane probed using HIVIgG as above. Treatment of soluble HIFV with various temperatures did not cause any dissociation of trimers into monomers or dimers as is evident with gp140R2 (see FIG. 39). In addition, treatment with SDS, which caused dissociation of gp140R2 trimer into monomers and degradation productions, had little effect on solHIFV, suggesting that solHIFV is more stable than soluble gp140R2.

### Example 17

### Foamy Virus TM Protein Expression Construct

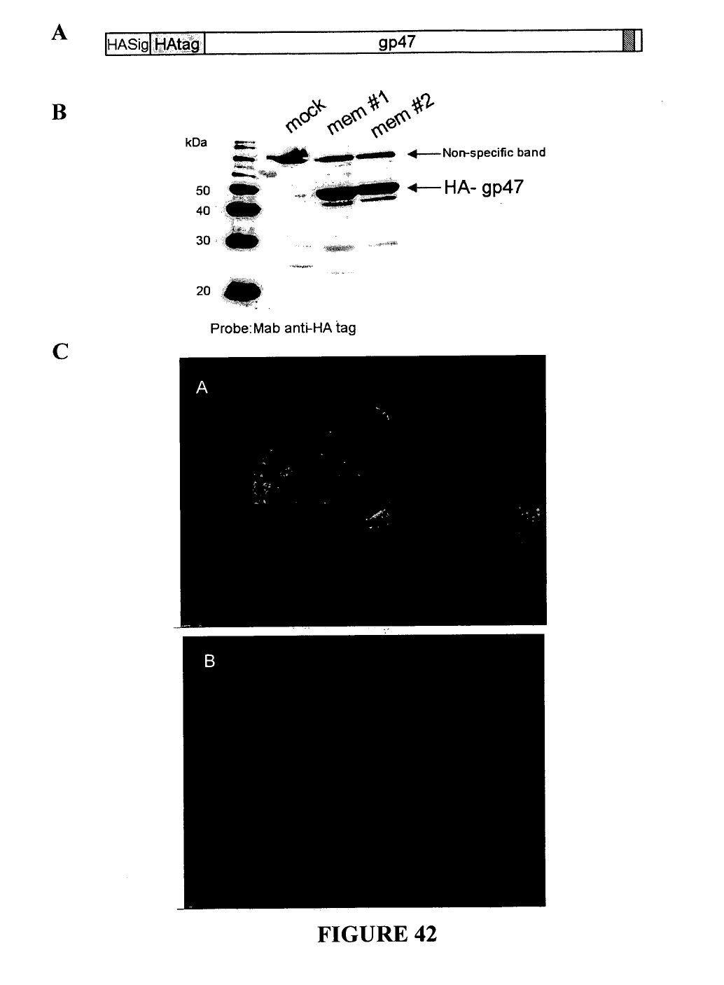

The ability of Foamy Virus to form envelope only VLPs is inherent in the TM protein subunit of the FV envelope polyprotein (SU/TM). The fusions of influenza virus HA 1 and gp120 of HIV-1 to gp47 described here were able to form VLPs without the requirement of the FV leader peptide. This would suggest that the TM subunit contains the necessary information for particle formation. In order to test this a TM protein expression construct was made with an N-terminal HA tag for antibody detection of the TM protein. A synthetic gene encoding the HA signal sequence and the HA antibody tag (YPYDVPDYA) fused to the N-terminus of gp47 with a 5′ Xho 1 and 3′ Not site was obtained from Genescript and codon optimised for mammalian expression. The insert was cloned into the pCI neo vector. The HA signal sequence is predicted to be cleaved between VKS-YP according to Signal P 3.0 server (schematic FIG. 42A).

### Example 18

### Expression of HAtag-gp47 Construct

To assess expression of the HAtag-gp47 construct (nucleic acid SEQ ID NO: 27, amino acid SEQ ID NO: 28) the plasmid was transfected into 293T cells and expression assessed by Western blotting and by immunofluorescence with monoclonal anti-HA tag (Abcam 16918) at 1 μg/ml. A crude membrane fraction was obtained from transfected cells by freeze-thawing the cells, centrifugation to remove the cytosol and solubilisation of the pelleted membrane proteins with 1% NP-40. The membrane fraction of mock and transfected cells was run on a 12% SDS-PAGE and Western blotted using anti-HA tag MAb. A band at the expect size of approximately 47 kDa was detected in membrane fraction (FIG. 42B). Mock and SigHA-HAtag-gp47 transfected 293T cells grown on glass coverslips were fixed in methanol day 2 post transfection and then stained with anti-HA tag MAb followed by anti-mouse IgG ALEXA 488 (green) and the nuclei stained red with propidium iodine. HAtag-gp47 is expressed in transfected cells although the expression appears to accumulate in the perinuclear region (FIG. 42C).

### Example 19

### VLP Formation by Optiprep Gradient Sedimentation

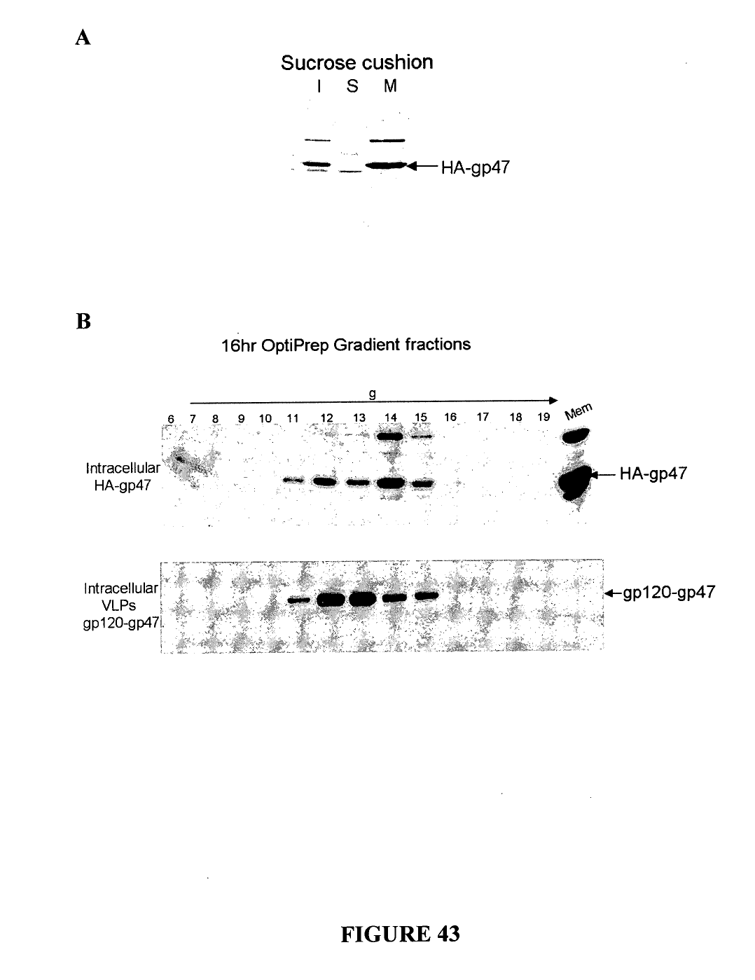

The media and cytosol fraction of cells transfected with SigHA-HAtag-gp47 was applied to a 20%-70% sucrose cushion and centrifuged at 38,000 rpm for 3 hours in a SW41 rotor. The interface was collected and 100 μl was methanol precipitated and assessed by SDS-PAGE and Western blotting with anti-HA tag MAb. FIG. 43A shows a Western blot of intracellular VLPs (I) and secreted VLPs(S) after sedimentation and soluble membrane protein (M). HA-gp47 was detected in the cytosol fraction but not the secreted (media) sample. The cytosol fraction of gp120-FP-gp47 transfected cells and the remaining cytosol fraction of SigHA-HAtag-gp47 transfected cells was applied to a 20-50% OptiPrep gradient and centrifuged at 38,000 rpm for 16 hours in an SW41 rotor. 500 μl fractions were collected and 150 μl methanol precipitated and assessed by SDS-PAGE and Western blotting with HIVIgG or anti-HA tag MAb. FIG. 43B shows that HA-gp47 is forming VLPs that sediment in the gradient, although slightly further down the gradient than gp120-FP-gp47 VLPs.

Many modifications will be apparent to those skilled in the art without departing from the scope of the present invention.

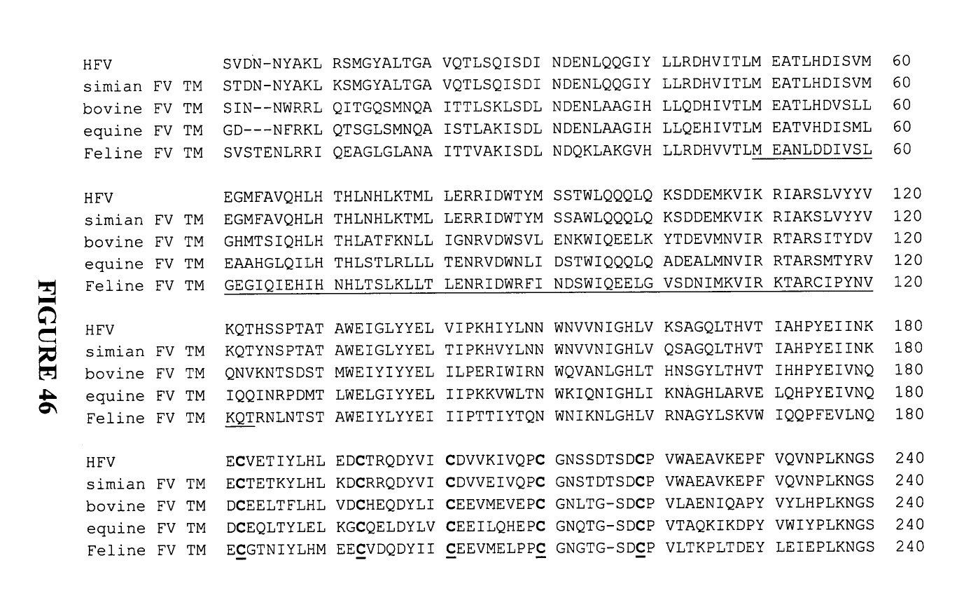

TABLE 1
Summary of sequence identifiers

| Sequence ID NO: | Description |
|---|---|
| SEQ ID NO: 1 | nucleotide sequence of HFV(SFVhu) (NC001736.1) |
| SEQ ID NO: 2 | nucleotide sequence of HFV (SFVhu) gp47 (NC001736.1) |
| SEQ ID NO: 3 | amino acid sequence of HFV (SFVhu) gp47 (NC001736.1) |
| SEQ ID NO: 4 | nucleotide sequence of HFV Leader Peptide (LP) sequence |
| | (NC001736.1) |
| SEQ ID NO: 5 | amino acid sequence of HFV Leader Peptide (LP) (NC001736.1) |
| SEQ ID NO: 6 | amino acid sequence of HFV (SFVhu) gp47 with deletion of |
| | cytoplasmic domain (NC001736.1) |
| SEQ ID NO: 7 | nucleotide sequence of H5NI HA complete A/Vietnam/3028/2004 |
| | (WHO) |
| SEQ ID NO: 8 | amino acid sequence of H5N1 HA A/Vietnam/3028/2004 |
| SEQ ID NO: 9 | nucleotide sequence of HA1-gp47 fusion |
| SEQ ID NO: 10 | amino acid sequence of HA1-gp47 fusion |
| SEQ ID NO: 11 | nucleotide sequence of LPHA1-gp47 |
| SEQ ID NO: 12 | amino acid sequence of LPHA1-gp47 fusion |
| SEQ ID NO: 13 | nucleotide sequence of HA1-gp47ΔCD fusion |
| SEQ ID NO: 14 | amino acid sequence of HA1-gp47ΔCD fusion |
| SEQ ID NO: 15 | nucleotide sequence of HA1-gp47 Codon Optimised |
| SEQ ID NO: 16 | nucleotide sequence of HA1-gp47 fusion insert |
| SEQ ID NO: 17 | nucleotide sequence of LPHA1-gp47 insert |
| SEQ ID NO: 18 | nucleotide sequence of gp47ΔCD insert |
| SEQ ID NO: 19 | nucleotide sequence of HIV-1 R2 envelope (AF128126) |
| SEQ ID NO: 20 | amino acid sequence of HIV R2 envelope (AF128126) |
| SEQ ID NO: 21 | nucleotide sequence of HIV-1 R2 gp120-FP-gp47ΔCD Codon |
| | Optimised |
| SEQ ID NO: 22 | amino acid sequence of HIV-1 R2 gp120-FP-gp47ΔCD |
| SEQ ID NO: 23 | nucleotide sequence of gp120-FP-gp47ΔCD insert |
| SEQ ID NO: 24 | amino acid sequence of N-terminal heptad repeat domain |
| SEQ ID NO: 25 | nucleotide sequence of gp120-FP-gp47ΔMSD/CD for soluble HIFV |
| | trimer construct |
| SEQ ID NO: 26 | amino acid sequence of HIV-1 gp120-FP-gp47ΔMSD |
| SEQ ID NO: 27 | nucleotide sequence of HASigHAtag-gp47 CO gene |
| SEQ ID NO: 28 | amino acid sequence of HAsigHAtag-gp47 |
| SEQ ID NO: 29 | amino acid sequence of HIV-1 membrane proximal region extending |
| | into C-terminal heptad repeat |
| SEQ ID NO: 30 | amino acid sequence 1 to 417 of FV TM set out in FIG. 45 |
| SEQ ID NO: 31 | amino acid sequence of FV TM fusion peptide (aa 1-48) |
| SEQ ID NO: 32 | amino acid sequence of cysteine rich region of FV TM (aa 123-296) |
| SEQ ID NO: 33 | amino acid sequence of C-terminal α-helical region FV TM (aa 377-404) |
| SEQ ID NO: 34 | amino acid sequence of membrane spanning domain FV TM (aa 405-417) |
| SEQ ID NO: 35 | amino acid sequence of cytoplasmic tail FV TM (aa 405-417) |
| SEQ ID NO: 36 | amino acid sequence of N-terminal heptad repeat domain, C-terminal |
| | α-helical domain FV TM (aa 49-373) |
| SEQ ID NO: 37 | amino acid sequence of N-terminal heptad repeat domain and cysteine- |
| | rich region FV TM (aa 49-296) |

TABLE 2
Suitable naturally occurring proteogenic amino acids

| Amino Acid | one letter code | three letter code |
|---|---|---|
| L-alanine | A | Ala |
| L-arginine | R | Arg |
| L-asparagine | N | Asn |
| L-aspartic acid | D | Asp |
| L-cysteine | C | Cys |
| L-glutamine | Q | Gln |
| L-glutamic acid | E | Glu |
| glycine | G | Gly |
| L-histidine | H | His |
| L-isoleucine | I | Ile |
| L-leucine | L | Leu |
| L-lysine | K | Lys |
| L-methionine | M | Met |
| L-phenylalanine | F | Phe |
| L-proline | P | Pro |
| L-serine | S | Ser |
| L-threonine | T | Thr |
| L-tryptophan | W | Trp |
| L-tyrosine | Y | Tyr |
| L-valine | V | Val |

TABLE 3
Amino acid sub-classification

| Sub-Classes | Amino Acids |
|---|---|
| Acidic | Aspartic acid, Glutamic acid |
| Basic | Noncyclic: Arginine, Lysine; Cyclic: Histidine |
| Charged | Aspartic acid, Glutamic acid, Arginine, Lysine, Histidine |
| Small | Glycine, Serine, Alanine, Threonine, Proline |
| Polar/neutral | Asparagine, Histidine, Glutamine, Cysteine, Serine, |
| | Threonine |
| Polar/large | Asparagine, Glutamine |
| Hydrophobic | Tyrosine, Valine, Isoleucine, Leucine, Methionine, |
| | Phenylalanine, Tryptophan |
| Aromatic | Tryptophan, Tyrosine, Phenylalanine |
| Residues that | Glycine and Proline |
| influence chain | |
| orientation | |

TABLE 4
Exemplary and Preferred Amino Acid Substitutions

| Original | | Preferred |
|---|---|---|
| Residue | Exemplary Substitutions | Substitutions |
| Ala | Val, Leu, Ile | Val |
| Arg | Lys, Gln, Asn | Lys |
| Asn | Gln, His, Lys, Arg | Gln |
| Asp | Glu | Glu |
| Cys | Ser | Ser |
| Gln | Asn, His, Lys, | Asn |
| Glu | Asp, Lys | Asp |
| Gly | Pro | Pro |
| His | Asn, Gln, Lys, Arg | Arg |
| Ile | Leu, Val, Met, Ala, Phe, Norleu | Leu |
| Leu | Norleu, Ile, Val, Met, Ala, Phe | Ile |
| Lys | Arg, Gln, Asn | Arg |
| Met | Leu, Ile, Phe | Leu |
| Phe | Leu, Val, Ile, Ala | Leu |
| Pro | Gly | Gly |
| Ser | Thr | Thr |
| Thr | Ser | Ser |
| Trp | Tyr | Tyr |
| Tyr | Trp, Phe, Thr, Ser | Phe |
| Val | Ile, Leu, Met, Phe, Ala, Norleu | Leu |

TABLE 5
List of abbreviations

Abbreviation

| | |
|---|---|
| HA | haemagglutinin |
| EGS | ethylene glycolbis(succinimidylsuccinate |
| VLP | virus-like particle |
| SVP | sub-viral particle |
| LP | leader peptide of FV envelope protein |
| SU | surface protein subunit of FV envelope protein |
| TM | transmembrane protein subunit of FV envelope protein |
| FP | fusion peptide |
| FV | foamy virus |
| HFV | human foamy virus |
| RSV | respiratory syncytial virus |
| CD | cytoplasmic domain |
| gp | glycoprotein |
| HIV | human immunodeficiency virus |
| SDS-PAGE | sodium dodecyl sulfate polyacrylamide gel electrophoresis |
| MAb | monoclonal antibody |
| MSD/TMD | membrane spanning or transmembrane domain |
| MPR | membrane proximal region |
| POI | Protein/s or peptide/s of interest |

### BIBLIOGRAPHY

Altschul et al., Nucl. Acids Res., 25: 3389-3402, 1997
Arkin and Youvan, Proc. Natl. Acad. Sci. USA, 89: 7811-7815, 1992
Atherton and Shephard, Peptide Synthesis. In Nicholson ed, Synthetic Vaccines, published by Blackwell Scientific Publications, Chapter 9
Ausubel et al., Cell Immunol., 193(1): 99-107, 1999
Ausubel et al., Current Protocols in Molecular Biology, Green Pub. Associates and Wiley-Interscience, New York, 1988
Ausubel et al., Current Protocols in Molecular Biology, John Wiley & Sons Inc, Chapter 15 and Unit 19.3, 1994-1998
Ausubel et al., Current Protocols in Molecular Biology, John Wiley & Sons Inc, Chapters 10 and 16, 1994
Baker et al., Biophys. J., 60: 1445-1456, 1991
Bandyopadhyay and Temin, Mol. Cell. Biol. 4: 749-754, 1984
Bansal et al., J. Virol. 74(6): 2949-2954, 2000
Berglund et al., Biotechnology 11: 916-920, 1993
Berkner et al., BioTechniques 6; 616-629, 1988
Berkner, Curr. Top. Microbiol. Immunol. 158: 39-66, 1992
Breakefield and Geller, Mol. Neurobiol. 1: 339-371, 1987
Buchschacher and Panganiban, J. Virol. 66: 2731-2739, 1982
Chambers et al., Journal of General Virology 71: 3075-3080, 1990
Coligan et al., Current Protocols in Protein Science, John Wiley & Sons, Inc., Chapters 1, 5 and 6, 1995-1997
Colowick and Kaplan, eds., Methods In Enzymology, Academic Press, Inc.
Dayhoff et al., Atlas of Protein Sequence and Structure, Natl. Biomed. Res. Found., Washington, D.C., 1978
Dayhoff ed., Atlas of protein sequence and structure, National Biomedical Research Foundation, Washington D.C., Vol. 5, pp. 345-358
Delgrave et al., Protein Engineering, 6: 327-331, 1993
Deveraux et al., Nucleic Acids Research 12: 387-395, 1984
Fields and Knipe, eds, Fundamental Virology, 2nd Edition, 1991
Fields et al., eds, Virology, 3rd Edition, Lippincott-Raven, Philadelphia, Pa., 1996
Fink et al., Ann. Rev. Neurosci. 19: 265-287, 1996
Fink et al., Hum. Gene Ther. 3: 11-19, 1992
Freese et al., Biochem. Pharmacol. 40: 2189-2199, 1990
Goepfert et al., J. Virol. 73: 7210-7217, 1999
Gonnet et al., Science, 256(5062): 1443-1445, 1992
Gorziglia and Kapikian, J. Virol. 66: 4407-4412, 1992
Hagensee et al., J. Virol., 68: 4503-4505, 1994
Hedestam et al., Nature Reviews Microbiology 6: 143-155, 2008
Helseth et al., J. Virol. 64: 2416-2420, 1990
Johnson et al., J. Virol. 66: 2952-2965, 1992
Joklik ed., Virology, 3rd Edition, 1988
Klingmüller and Schaller, J. Virol. 67(12): 7414-22, 1993
Kozak and Shatkin, Methods Enzymol, 60: 360-375, 1979
Kozak, Biochimie., 76(9):815-821, 1994
Kozak, J Cell Biol, 108(2): 229-241, 1989
Kozak, Mamm Genome, 7(8): 563-74, 1996
Kunkel et al., Methods in Enzymol., 154: 367-382, 1987
Kunkel, Proc. Natl. Acad. Sci. USA, 82: 488-492, 1985
Lui et al., PLoS Pathogens 4(7): 1-22, 2008
Madzak et al., J. Gen. Virol. 73: 1533-1536, 1992
Mann and Baltimore, J. Virol. 54: 401-407, 1985
Margolskee, Curr. Top., Microbiol. Immunol. 158: 67-95, 1992
Miller et al., J. Virol. 62: 4337-4345, 1988
Miller et al., Mol. Cell. Biol. 5: 431-437, 1985
Miller, Curr. Top. Microbiol. Immunol. 158: 1-24, 1992
Moss, Curr. Top. Microbiol. Immunol. 158: 25-38, 1992
Moss, Proc. Natl. Acad. Sci. USA 93: 11341-11348, 1996
Muzyczka, Curr. Top. Microbiol. Immunol. 158: 97-129, 1992
Naldini et al., Science 272: 263-267, 1996
Newton and Graham eds., PCR, Introduction to Biotechniques Series, 2nd ed., Springer Verlag, 1997,
Ohi et al., Gene 89: 279-282, 1990
Page et al., J. Virol. 64: 5270-5276, 1990
Pantophlet and Burton, Annu. Rev. Immunol. 24: 739-769, 2006
Petropoulos et al., J. Viol. 66: 3391-3397, 1992
Quantin et al., Proc. Natl. Acad. Sci. USA 89: 2581-2584, 1992
Quinnan et al., J. Virol. 79(6): 3358-3369, 2005
Ream et al., eds., Molecular Biology Techniques: An Intensive Laboratory Course, Academic Press, 1998
Remington's Pharmaceutical Sciences, 18th Ed., Mack Publishing, Company, Easton, Pa., U.S.A., 1990
Roberge et al., Science, 269(5221): 202-204, 1995
Rose et al., A Laboratory Course Manual, Cold Spring Harbor Laboratory, Cold Spring Harbor, N.Y., 1990
Rosenfeld et al., Cell 68: 143-155, 1992
Russell and Hirata, Nature Genetics 18: 323-328, 1998
Sambrook et al., Molecular Cloning: A Laboratory Manual, Cold Spring Harbor Laboratory, Cold Spring Harbor, N.Y., Sections 1.101 to 1.104 and Sections 16 and 17, 1989
Schneider et al., Nature Genetics 18: 180-183, 1998
Sharp and Cowe, Yeast, 7: 657-678, 1991
Shaw et al., J. Virol. 77(4): 2338-48, 2003
Shimada et al., J. Clin. Invest. 88: 1043-1047, 1991
Sorge et al., Mol. Cell. Biol. 4: 1730-1737, 1984
Stange et al., J. Virol. 82(20): 9858-69, 2008
Stanke et al., J. Virol. 79(24): 15074-15083. 2005
Stratford-Perricaudet et al., Hum. Gene Ther. 1: 241-256, 1990
Tobaly-Tapiero et al., J. Virol. 74(9): 4064-4073, 2000
Wang and Mulligan, J. Gen. Virol. 80: 245-254, 1999
Watson et al., Molecular Biology of the Gene, Fourth Edition, Benjamin/Cummings, Menlo Park, Calif., 1987
Weir and Blackwell, eds., Handbook of Experimental Immunology, Vols. I-IV, Blackwell Scientific Publications, 1986
Wilk et al., J. Virol. 74(6): 2885-2887, 2000
Wilkinson et al., Nucleic Acids Res. 20: 2233-2239, 1992
Zhang et al., PNAS 104(24): 10193-10198, 2007
Zubay, Biochemistry, third edition, Wm.C. Brown Publishers, 1993

1. A chimeric protein comprising a heterologous (non-FV) polypeptide or peptide of interest and at least a trimer forming or a trimer and VLP forming portion of a transmembrane protein of foamy virus envelope protein (FV TM).

2. The chimeric protein of claim 1 wherein the polypeptide or peptide of interest is an antigen of a pathogenic or other antigen against which an immune response is sought.

3. The chimeric protein of claim 1 wherein the polypeptide or peptide of interest is an antigen of a viral envelope protein or a viral pathogen.

4. The chimeric protein of claim 3 wherein the non-foamy virus virus envelope protein is selected from HIV-1gp120, HIV-2gp125, HA of influenza virus, SARS S1 protein, RSV F2 protein, and Dengue Virus E protein.

5. The chimeric protein of claim 1 wherein the trimer and/or VLP forming portion of transmembrane protein of foamy virus envelope protein comprises:
i) full length foamy virus transmembrane protein;
ii) foamy virus transmembrane protein absent a functional cytoplasmic domain;
iii) foamy virus transmembrane protein absent a functional cytoplasmic domain and transmembrane domain;
iv) foamy virus ectodomain comprising N-terminal heptad repeat region and cysteine rich region between N-terminal heptad repeat region and C-terminal α-helical region;
v) N-terminal heptad repeat region;
vi) a functional variant of any one of i) to v); or
vii) any one of i) to vi) lacking an FV fusion peptide domain.

6. The chimeric protein of claim 1 in the form of a trimer or a complex comprising a trimer.

7. A viral-like particle comprising a trimeric chimeric protein according to claim 1.

8. A nucleic acid encoding the chimeric protein according to claim 1.

9. A pharmaceutical composition comprising the chimeric protein according to claim 1, a viral-like particle comprising a trimeric chimeric protein according to claim 1 or a nucleic acid encoding the chimeric protein according to claim 1.

10. A method for inducing an immune response in a subject in need comprising administering a chimeric protein according to claim 1, a viral-like particle comprising a trimeric chimeric protein according to claim 1 or a nucleic acid encoding the chimeric protein according to claim 1.

11. A method for producing an antibody comprising immunising a non-human animal or screening expression products of a library of human immunoglobulin genes with a chimeric protein according to claim 1, a viral-like particle comprising a trimeric chimeric protein according to claim 1or a nucleic acid encoding the chimeric protein according to claim 1 and isolating an antibody that binds specifically to the antigen.

12. An antibody produced by the method of claim 11 or an antigen-binding fragment or a chimeric, human or humanised form thereof.

13. A method of screening antibodies or other agents that specifically bind to a trimeric viral envelope polypeptide, comprising contacting a sample or solution comprising an antibody or other putative binding agent with a chimeric protein according to claim 1 and determining binding relative to controls.
